SEQUENCE LISTING (42 sequences)

SEQ ID NO: 1 - protein, 5 aa, Artificial Sequence
  Synthetic peptide
GYWWT

SEQ ID NO: 2 - protein, 16 aa, Artificial Sequence
  Synthetic peptide
EINPSDTTNYNPSLKS

SEQ ID NO: 3 - protein, 20 aa, Artificial Sequence
  Synthetic peptide
ARGRSTYGASTFYNYYYFDS

SEQ ID NO: 4 - protein, 11 aa, Artificial Sequence
  Synthetic peptide
RASQKIDRWLA

SEQ ID NO: 5 - protein, 7 aa, Artificial Sequence
  Synthetic peptide
KASNLES

SEQ ID NO: 6 - protein, 9 aa, Artificial Sequence
  Synthetic peptide
QQYQNYPYT

SEQ ID NO: 7 - protein, 126 aa, Artificial Sequence
  Synthetic polypeptide
QVQLQQWGAGLLKPSETLSLTCAVYGGSFSGYWWTWIRQSPEKGLEWIGEINPSDTTNYNPSLKSRVAISKDSSKNQFSLRLSSVTAADTALYFCARGRSTYGASTFYNYYYFDSWGQGSRVVVSS

SEQ ID NO: 8 - protein, 108 aa, Artificial Sequence
  Synthetic polypeptide
DIQMTQSPSTLSASVGDTITITCRASQKIDRWLAWYQQKPGKAPKLLIYKASNLESGVPPRLSGTGSGTDFTLTIKSLQPDDFATYFCQQYQNYPYTFGPGTKLEMRR

SEQ ID NO: 9 - protein, 456 aa, Artificial Sequence
  Synthetic polypeptide
QVQLQQWGAGLLKPSETLSLTCAVYGGSFSGYWWTWIRQSPEKGLEWIGEINPSDTTNYNPSLKSRVAISKDSSKNQFSLRLSSVTAADTALYFCARGRSTYGASTFYNYYYFDSWGQGSRVVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  Synthetic polypeptide
DIQMTQSPSTLSASVGDTITITCRASQKIDRWLAWYQQKPGKAPKLLIYKASNLESGVPPRLSGTGSGTDFTLTIKSLQPDDFATYFCQQYQNYPYTFGPGTKLEMRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, Artificial Sequence
  Synthetic peptide
GSGIH

SEQ ID NO: 12 - protein, 19 aa, Artificial Sequence
  Synthetic peptide
HIRSKSDGSATLYAASVRG

SEQ ID NO: 13 - protein, 19 aa, Artificial Sequence
  Synthetic peptide
ARYRRVAVAGYTYYYYMDV

SEQ ID NO: 14 - protein, 11 aa, Artificial Sequence
  Synthetic peptide
RASQSIDSYLN

SEQ ID NO: 15 - protein, 7 aa, Artificial Sequence
  Synthetic peptide
AASSLQS

SEQ ID NO: 16 - protein, 9 aa, Artificial Sequence
  Synthetic peptide
QQSHSLPRT

SEQ ID NO: 17 - protein, 128 aa, Artificial Sequence
  Synthetic polypeptide
EVQLVESGGGLVHPGESLRLSCAASGFSLSGSGIHWVRQASGKGLEWLGHIRSKSDGSATLYAASVRGRFIISRDDSTNTAYLHMSSLRNEDTAVYYCARYRRVAVAGYTYYYYMDVWGKGSTVSVSS

SEQ ID NO: 18 - protein, 108 aa, Artificial Sequence
  Synthetic polypeptide
DIQMTQSPSSLSASVGDRVTITCRASQSIDSYLNWYQQKPGKAPKLLIYAASSLQSGVPSRFSGSGFGTDFTLTISRLQPEDFATYCCQQSHSLPRTFGQGTKVEIKR

SEQ ID NO: 19 - protein, 458 aa, Artificial Sequence
  Synthetic polypeptide
EVQLVESGGGLVHPGESLRLSCAASGFSLSGSGIHWVRQASGKGLEWLGHIRSKSDGSATLYAASVRGRFIISRDDSTNTAYLHMSSLRNEDTAVYYCARYRRVAVAGYTYYYYMDVWGKGSTVSVSSASTKGPSVFPLAPCSRSTSESTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEEYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Artificial Sequence
  Synthetic polypeptide
DIQMTQSPSSLSASVGDRVTITCRASQSIDSYLNWYQQKPGKAPKLLIYAASSLQSGVPSRFSGSGFGTDFTLTISRLQPEDFATYCCQQSHSLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 378 bp, Artificial Sequence
  Synthetic polynucleotide
caggtgcagctacagcagtggggcgcaggactgttgaagccttcggagaccctgtccctcacttgcgctgtctatggtgggtccttcagtggttactggtggacttggatccgccagtccccagagaagggcctggagtggattggcgaaatcaatcctagtgacaccaccaattacaacccgtccctcaagagtcgagtcgccatatcaaaggactcatccaagaaccaattctccctgcggctgtcgtctgtgaccgccgcggacacggctctctatttctgtgcgagaggccgttccacctacggggccagcactttctacaactactactactttgactcttggggccagggatcgcgggtcgtcgtctcttca

SEQ ID NO: 22 - DNA, 324 bp, Artificial Sequence
  Synthetic polynucleotide
gacatccagatgacccagtctccatccaccctgtctgcttctgtcggagacacaattaccatcacttgccgggccagtcagaagattgataggtggttggcctggtatcagcagaaaccaggcaaagcccctaaacttctcatatataaggcctctaatttagagagtggggtcccacctaggctcagcggcactggatctgggacagacttcactctcactatcaagagcctgcagccggatgattttgcaacgtatttctgtcaacagtatcagaattacccctacacttttggcccggggaccaagctggagatgagacga

SEQ ID NO: 23 - DNA, 384 bp, Artificial Sequence
  Synthetic polynucleotide
gaggtgcagctggtggagtctgggggaggcttggtccaccctggggagtccctgagactctcctgtgcagcctctgggtttagtttgagtggctctggtatacactgggtccgccaggcttccggaaaagggctggagtggcttggccatattagaagcaaaagtgacggttccgcgacattgtatgctgcgtcggtgagaggcaggttcattatctccagagatgattcaacgaatacggcgtatctgcacatgagcagcctgagaaacgaggacacggccgtctattactgcgctagatacagacgagtggcagtggcaggttacacctactactactacatggacgtctggggcaaagggtccacggtctccgtctcctca

SEQ ID NO: 24 - DNA, 324 bp, Artificial Sequence
  Synthetic polynucleotide
gacatccagatgacccagtctccatcctccctgtctgcatctgtaggagacagagtcaccatcacttgccgggcaagtcagagcattgacagctatttaaattggtatcagcagaaaccagggaaagcccctaagctcctgatctatgctgcatccagtttgcaaagtggggtcccatcaaggttcagtggcagtggatttgggacagatttcactctcaccatcagcaggctgcaacctgaggattttgcaacttactgctgtcaacagagtcacagtctcccccggacgttcggccaagggaccaaggtggaaatcaaacga

SEQ ID NO: 25 - DNA, 1368 bp, Artificial Sequence
  Synthetic polynucleotide
caggtgcagctacagcagtggggcgcaggactgttgaagccttcggagaccctgtccctcacttgcgctgtctatggtgggtccttcagtggttactggtggacttggatccgccagtccccagagaagggcctggagtggattggcgaaatcaatcctagtgacaccaccaattacaacccgtccctcaagagtcgagtcgccatatcaaaggactcatccaagaaccaattctccctgcggctgtcgtctgtgaccgccgcggacacggctctctatttctgtgcgagaggccgttccacctacggggccagcactttctacaactactactactttgactcttggggccagggatcgcgggtcgtcgtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggaggagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 26 - DNA, 642 bp, Artificial Sequence
  Synthetic polynucleotide
gacatccagatgacccagtctccatccaccctgtctgcttctgtcggagacacaattaccatcacttgccgggccagtcagaagattgataggtggttggcctggtatcagcagaaaccaggcaaagcccctaaacttctcatatataaggcctctaatttagagagtggggtcccacctaggctcagcggcactggatctgggacagacttcactctcactatcaagagcctgcagccggatgattttgcaacgtatttctgtcaacagtatcagaattacccctacacttttggcccggggaccaagctggagatgagacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 27 - DNA, 1374 bp, Artificial Sequence
  Synthetic polynucleotide
gaggtgcagctggtggagtctgggggaggcttggtccaccctggggagtccctgagactctcctgtgcagcctctgggtttagtttgagtggctctggtatacactgggtccgccaggcttccggaaaagggctggagtggcttggccatattagaagcaaaagtgacggttccgcgacattgtatgctgcgtcggtgagaggcaggttcattatctccagagatgattcaacgaatacggcgtatctgcacatgagcagcctgagaaacgaggacacggccgtctattactgcgctagatacagacgagtggcagtggcaggttacacctactactactacatggacgtctggggcaaagggtccacggtctccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggaggagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 28 - DNA, 642 bp, Artificial Sequence
  Synthetic polynucleotide
gacatccagatgacccagtctccatcctccctgtctgcatctgtaggagacagagtcaccatcacttgccgggcaagtcagagcattgacagctatttaaattggtatcagcagaaaccagggaaagcccctaagctcctgatctatgctgcatccagtttgcaaagtggggtcccatcaaggttcagtggcagtggatttgggacagatttcactctcaccatcagcaggctgcaacctgaggattttgcaacttactgctgtcaacagagtcacagtctcccccggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 29 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 30 - protein, 18 aa, Homo sapiens
MKLSLVAAMLLLLSAARA

SEQ ID NO: 31 - protein, 19 aa, Artificial Sequence
  Synthetic peptide
MEWSWVFLFFLSVTTGVHS

SEQ ID NO: 32 - protein, 20 aa, Artificial Sequence
  Synthetic peptide
MGVPTQVLGLLLLWLTDARC

SEQ ID NO: 33 - protein, 4 aa, Artificial Sequence
  Synthetic peptide
GGGS

SEQ ID NO: 34 - protein, 4 aa, Artificial Sequence
  Synthetic peptide
SGGG

SEQ ID NO: 35 - protein, 5 aa, Artificial Sequence
  Synthetic peptide
GGGGS

SEQ ID NO: 36 - protein, 5 aa, Artificial Sequence
  Synthetic peptide
SGGGG

SEQ ID NO: 37 - protein, 6 aa, Artificial Sequence
  Synthetic peptide
GGGGGS

SEQ ID NO: 38 - protein, 6 aa, Artificial Sequence
  Synthetic peptide
SGGGGG

SEQ ID NO: 39 - protein, 7 aa, Artificial Sequence
  Synthetic peptide
GGGGGGS

SEQ ID NO: 40 - protein, 7 aa, Artificial Sequence
  Synthetic peptide
SGGGGGG

SEQ ID NO: 41 - DNA, 57 bp, Artificial Sequence
  Synthetic oligonucleotide
atggaatggagctgggtctttctcttcttcctgtcagtaactacaggtgtccactcc

SEQ ID NO: 42 - DNA, 60 bp, Artificial Sequence
  Synthetic oligonucleotide
atgggtgtgcccactcaggtcctggggttgctgctgctgtggcttacagatgccagatgt

(19) United States
(12) Patent Application Publication
(10) Pub. No.: US 2021/0301002 A1
(43) Pub. Date: Sep. 30, 2021

(54) ANTI-HSPA5 ANTIBODIES AND USES THEREOF

(71) Applicants: Biogen MA Inc., Cambridge, MA (US); The Regents of the University of Colorado, a body corporate, Denver, CO (US)

(72) Inventors: Jeffrey L. Bennett, Denver, CO (US); Richard M. Ransohoff, Boston, MA (US); Fumitaka Shimizu, Yamaguchi (JP); Kristin L. Schaller, Denver, CO (US)

(73) Assignees: Biogen MA Inc., Cambridge, MA (US); The Regents of the University of Colorado, a body corporate, Denver, CO (US)

(21) Appl. No.: 17/182,426

(22) Filed: Feb. 23, 2021

Related U.S. Application Data
(63) Continuation of application No. 16/319,715, filed on Jan. 22, 2019, now abandoned.
(60) Provisional application No. 62/502,188, filed on May 5, 2017.
(60) Provisional application No. 62/366,452, filed on Jul. 25, 2016.

(51) Int. Cl. C07K 16/18; A61K 9/00; C12N 15/85; G01N 33/68

(52) U.S. Cl. CPC C07K 16/18; A61K 9/0019; A61K 45/06; G01N 33/6854; C12N 15/85

(57) ABSTRACT

This disclosure provides anti-HSPA5 antibodies. Also featured is a method for delivering an agent into the brain, spinal cord, or other component of the central nervous system. In addition, methods of identifying a human subject at risk of relapse of neuromyelitis optica (NMO) or neuropsychiatric systemic lupus erythematosus (NP-SLE) are disclosed, as are methods of determining the severity of an attack of NMO or NP-SLE, and methods of diagnosing NP-SLE.

### CROSS-REFERENCE TO RELATED APPLICATIONS

This applications claims the benefit of priority of U.S. Provisional Application No. 62/366,452 filed Jul. 25, 2016 and U.S. Provisional Application No. 62/502,188 filed May 5, 2017, the content of both of which are incorporated by reference herein in their entireties.

### STATEMENT OF FEDERALLY SPONSORED RESEARCH

This invention was made with government support under Grant No. EY022936 awarded by the National Institutes of Health. The Government has certain rights in the invention.

### BACKGROUND

Antibody-based therapeutics have evolved as a major drug development pipeline for treating diseases of the central nervous system (CNS). There are abundant CNS drug targets for which antibody-mediated approaches have shown therapeutic effects in proof-of-principle studies in preclinical models. Unfortunately, poor blood-brain barrier (BBB) penetration of large molecules limits the utility of therapeutic antibodies for CNS disease, as only 0.1% of circulating antibodies typically reach the brain across the intact BBB (Yu Y J et al., Neurotherapeutics, 10: 459-72 (2013)). Therapeutic antibody transport across the BBB could be enhanced by taking advantage of receptor-mediated transcytosis (RMT) (Jones A R et al, Pharm Res., 24: 1759-71 (2007); Watts R J et al, Curr Opin Chem Biol., 17: 393-9 (2013)); however, abundance of RMT receptors in peripheral tissues and their potential roles in critical cellular function pose both efficacy and safety concerns for the development of many RMT-based approaches (Couch J A et al, Sci Transl Med., 5:183ra57 (2013)). Thus, there is a great unmet need for the discovery of novel mechanisms for manipulating BBB permeability that expand current approaches for enhancing CNS uptake of therapeutic antibodies and other drugs.

### SUMMARY

The invention is based at least in part on the discovery that Heat Shock Protein Family A (Hsp70) Member 5 (HSPA5) is localized on the cell surface of brain microvascular endothelial cells (BMECs) and is a target of anti-endothelial cell antibodies (AECA) in neuromyelitis optica (NMO) and neuropsychiatric systemic lupus erythematosus (NP-SLE) patients. This disclosure features anti-HSPA5 antibodies. Anti-HSPA5 antibodies are expected to serve as potential disease activity biomarkers in NMO and NP-SLE and modulate the severity of CNS injury. This disclosure also provides a platform technology for the delivery of any agent of interest (e.g., a therapeutic agent such as an antibody, other protein or nucleic acid therapeutic, or small molecule) to the central nervous system (i.e., brain, spinal cord). Specifically, anti-HSPA5 antibodies can also be employed to promote BBB transit of therapies (e.g., for CNS diseases).

In a first aspect, the disclosure features an isolated antibody or antigen-binding fragment thereof that specifically binds human heat shock protein family A (Hsp70) member 5 (human HSPA5), wherein the antibody or antigen-binding fragment thereof binds to the same epitope on human HSPA5 as a reference antibody, or competes with the reference antibody to bind to human HSPA5. The reference antibody comprises: a heavy chain variable region (VH) comprising VH complementarity determining regions 1, 2, and 3 with the amino acid sequences set forth in SEQ ID NO:1, SEQ ID NO:2, SEQ ID NO:3, respectively, and a light chain variable region (VL) comprising VL complementarity determining regions 1, 2, and 3 with the amino acid sequences set forth in SEQ ID NO:4, SEQ ID NO:5, and SEQ ID NO:6, respectively; or a VH comprising VH complementarity determining regions 1, 2, and 3 with the amino acid sequences set forth in SEQ ID NO:11, SEQ ID NO:12, SEQ ID NO:13, respectively, and a VL comprising VL complementarity determining regions 1, 2, and 3 with the amino acid sequences set forth in SEQ ID NO:14, SEQ ID NO:15, and SEQ ID NO:16, respectively.

In certain embodiments, the VH of the reference antibody consists of SEQ ID NO:7 and the VL of the reference antibody consists of SEQ ID NO:8. In other embodiments, the VH of the reference antibody consists of SEQ ID NO:17 and the VL of the reference antibody consists of SEQ ID NO:18.

In some embodiments, the reference antibody comprises a heavy chain and a light chain, wherein the heavy chain consists of SEQ ID NO:9 and the light chain consists of SEQ ID NO:10; or the heavy chain consists of SEQ ID NO:19 and the light chain consists of SEQ ID NO:20.

In certain embodiments, the antibody comprises a heavy chain of a human IgG isotype. In certain instances, the human IgG isotype is selected from the group consisting of IgG1, IgG2a, IgG2b, IgG3, and IgG4.

In certain embodiments, the antibody comprises a light chain of a human lambda isotype. In other embodiments, the antibody comprises a light chain of a human kappa isotype.

In some embodiments, the antigen-binding fragment is an scFv, an sc(Fv)2, an Fab, an F(ab)2, a diabody, a humanized nanobody, or a humanized Variable domain of New Antigen Receptor (VNAR).

In certain embodiments, the antibody or antigen-binding fragment is human or humanized.

In a second aspect, the disclosure provides an isolated antibody or antigen-binding fragment thereof that specifically binds human HSPA5, wherein the antibody or antigen-binding fragment thereof comprises: a heavy chain variable region (VH) comprising VH complementarity determining regions 1, 2, and 3 with the amino acid sequences set forth in SEQ ID NO:1, SEQ ID NO:2, SEQ ID NO:3, respectively, and a light chain variable region (VL) comprising VL complementarity determining regions 1, 2, and 3 with the amino acid sequences set forth in SEQ ID NO:4, SEQ ID NO:5, and SEQ ID NO:6, respectively; or a VH comprising VH complementarity determining regions 1, 2, and 3 with the amino acid sequences set forth in SEQ ID NO:11, SEQ ID NO:12, SEQ ID NO:13, respectively, and a VL comprising VL complementarity determining regions 1, 2, and 3 with the amino acid sequences set forth in SEQ ID NO:14, SEQ ID NO:15, and SEQ ID NO:16, respectively.

In some embodiments, the VH consists of an amino acid sequence that is at least 75% identical to the amino acid sequence set forth in SEQ ID NO:7 and the VL consists of an amino acid sequence that is at least 75% identical to the amino acid sequence set forth in SEQ ID NO:8; or the VH consists of an amino acid sequence that is at least 75% identical to the amino acid sequence set forth in SEQ ID NO:17 and the VL consists of an amino acid sequence that is at least 75% identical to the amino acid sequence set forth in SEQ ID NO:18.

In some embodiments, the VH consists of an amino acid sequence that is at least 95% identical to the amino acid sequence set forth in SEQ ID NO:7 and the VL consists of an amino acid sequence that is at least 95% identical to the amino acid sequence set forth in SEQ ID NO: 8, wherein the VH and VL are not identical to the amino acid sequence set forth in SEQ ID NOs: 7 and 8, respectively; or the VH consists of an amino acid sequence that is at least 95% identical to the amino acid sequence set forth in SEQ ID NO:17 and the VL consists of an amino acid sequence that is at least 95% identical to the amino acid sequence set forth in SEQ ID NO:18, wherein the VH and VL are not identical to the amino acid sequence set forth in SEQ ID NOs:17 and 18, respectively.

In certain embodiments, the VH consists of SEQ ID NO:7 and the VL consists of SEQ ID NO:8; or the VH consists of SEQ ID NO:17 and the VL consists of SEQ ID NO:18.

In some embodiments, the antibody or antigen-binding fragment comprises a heavy chain and a light chain, wherein the heavy chain consists of an amino acid sequence that is at least 75% identical to the amino acid sequence set forth in SEQ ID NO:9 and the light chain consists of an amino acid sequence that is at least 75% identical to the amino acid sequence set forth in SEQ ID NO:10; or the heavy chain consists of an amino acid sequence that is at least 75% identical to the amino acid sequence set forth in SEQ ID NO:19 and the light chain consists of an amino acid sequence that is at least 75% identical to the amino acid sequence set forth in SEQ ID NO:20.

In certain embodiments, the antibody comprises a heavy chain and a light chain, wherein: the heavy chain consists of an amino acid sequence that is at least 95% identical to the amino acid sequence set forth in SEQ ID NO:9 and the light chain consists of an amino acid sequence that is at least 95% identical to the amino acid sequence set forth in SEQ ID NO:10, wherein the heavy chain and light chain are not identical to the amino acid sequence set forth in SEQ ID NOs:9 and 10, respectively; or the heavy chain consists of an amino acid sequence that is at least 95% identical to the amino acid sequence set forth in SEQ ID NO:19 and the light chain consists of an amino acid sequence that is at least 95% identical to the amino acid sequence set forth in SEQ ID NO:20, wherein the heavy chain and light chain are not identical to the amino acid sequence set forth in SEQ ID NOs:19 and 20, respectively.

In some embodiments, the antibody comprises a heavy chain and a light chain, wherein: the heavy chain consists of SEQ ID NO:9 and the light chain consists of SEQ ID NO:10; or the heavy chain consists of SEQ ID NO:19 and the light chain consists of SEQ ID NO:20.

In certain embodiments, the antibody comprises a heavy chain of a human IgG isotype. In certain instances, the human IgG isotype is selected from the group consisting of IgG1, IgG2a, IgG2b, IgG3, and IgG4.

In certain embodiments, the antibody comprises a light chain of a human lambda isotype. In other embodiments, the antibody comprises a light chain of a human kappa isotype.

In some embodiments, the antigen-binding fragment is an scFv, an sc(Fv)2, an Fab, an F(ab)2, a diabody, a humanized nanobody, or a humanized Variable domain of New Antigen Receptor (VNAR).

In certain embodiments, the antibody or antigen-binding fragment is human or humanized.

In a third aspect, the disclosure features an expression vector comprising a promoter operably linked to a polynucleotide encoding a polypeptide comprising: (i) an immunoglobulin heavy chain variable region (VH) comprising VH complementarily determining regions (CDRs) 1, 2, and 3 with the amino acid sequences set forth in SEQ ID NOs:1-3, respectively, wherein the VH when paired with a light chain variable region (VL) comprising the amino acid sequence set forth in SEQ ID NO:8 binds human HSPA5; (ii) an immunoglobulin VL comprising VL CDRs 1, 2, and 3 with the amino acid sequences set forth in SEQ ID NOs:4-6, respectively, wherein the VL when paired with a VH comprising the amino acid sequence set forth in SEQ ID NO:7 binds to human HSPA5; (iii) an immunoglobulin VH comprising VH CDRs 1, 2, and 3 with the amino acid sequences set forth in SEQ ID NOs:11-13, respectively, wherein the VH when paired with a VL comprising the amino acid sequence set forth in SEQ ID NO:18 binds human HSPA5; or (iv) an immunoglobulin VL comprising VL CDRs 1, 2, and 3 with the amino acid sequences set forth in SEQ ID NOs:14-16, respectively, wherein the VL when paired with a VH comprising the amino acid sequence set forth in SEQ ID NO:17 binds to human HSPA5.

In certain embodiments, the promoter is a heterologous promoter. In some instances, the heterologous promoter is a cytomegalovirus, simian virus 40, or retroviral promoter. In certain instances, the heterologous promoter is the cytomegalovirus immediate early promoter.

In some embodiments, the expression vector comprises cytomegalovirus intron-A.

In some embodiments, the polypeptide encoded by the expression vector comprises a signal peptide. In certain instances, the signal peptide is a heterologous signal peptide.

In certain embodiments, the polypeptide encoded by the expression vector comprises: (i) an immunoglobulin heavy chain or fragment thereof comprising a VH with the amino acid sequence set forth in SEQ ID NO:7; (ii) an immunoglobulin light chain or fragment thereof comprising a VL with the amino acid sequence set forth in SEQ ID NO:8; (iii) an immunoglobulin heavy chain or fragment thereof comprising a VH with the amino acid sequence set forth in SEQ ID NO:17; or (iv) an immunoglobulin light chain or fragment thereof comprising a VL with the amino acid sequence set forth in SEQ ID NO:18.

In other embodiments, the polynucleotide comprises the nucleic acid sequence set forth in SEQ ID NO:21, SEQ ID NO:22, SEQ ID NO:23, or SEQ ID NO:24.

In certain embodiments, the expression vector is a plasmid, phage, virus, or retrovirus.

In a fourth aspect, the disclosure provides an expression vector comprising (a) a first polynucleotide encoding a first polypeptide comprising an immunoglobulin heavy chain or a fragment thereof comprising a heavy chain variable region (VH) comprising VH complementarity determining regions (CDRs) 1, 2, and 3 with the amino acid sequences set forth in SEQ ID NOs:1-3, respectively; and a second polynucleotide encoding a second polypeptide comprising an immunoglobulin light chain or a fragment thereof comprising a light chain variable region (VL) comprising VL CDRs 1, 2, and 3 with the amino acid sequences set forth in SEQ ID NOs:4-6, respectively, wherein the immunoglobulin heavy chain or fragment thereof when paired with the immunoglobulin light chain or fragment thereof forms an anti-human HSPA5 antibody or human HSPA5-binding fragment thereof; or (b) a first polynucleotide encoding a first polypeptide comprising an immunoglobulin heavy chain or a fragment thereof comprising a VH comprising VH CDRs 1, 2, and 3 with the amino acid sequences set forth in SEQ ID NOs:11-13, respectively; and a second polynucleotide encoding a second polypeptide comprising an immunoglobulin light chain or a fragment thereof comprising a VL comprising VL CDRs 1, 2, and 3 with the amino acid sequences set forth in SEQ ID NOs:14-16, respectively, wherein the immunoglobulin heavy chain or fragment thereof when paired with the immunoglobulin light chain or fragment thereof forms an anti-human HSPA5 antibody or human HSPA5-binding fragment thereof.

In some embodiments, the immunoglobulin heavy chain comprises a human IgG1 heavy chain constant region and the immunoglobulin light chain comprises a human lambda light chain constant region.

In other embodiments, the immunoglobulin heavy chain comprises a human IgG1 heavy chain constant region and the immunoglobulin light chain comprises a human kappa light chain constant region.

In certain embodiments, the VH consists of the amino acid sequence set forth in SEQ ID NO:7, and the VL consists of the amino acid sequence set forth in SEQ ID NO:8; or the VH consists of the amino acid sequence set forth in SEQ ID NO:17, and the VL consists of the amino acid sequence set forth in SEQ ID NO:18.

In some embodiments, the heavy chain consists of SEQ ID NO:9 and the light chain consists of SEQ ID NO:10; or the heavy chain consists of SEQ ID NO:19 and the light chain consists of SEQ ID NO:20.

In certain embodiments, the first polynucleotide comprises the nucleic acid sequence set forth in SEQ ID NO:21, and the second polynucleotide comprises the nucleic acid sequence set forth in SEQ ID NO:22; or the first polynucleotide comprises the nucleic acid sequence set forth in SEQ ID NO:23, and the second polynucleotide comprises the nucleic acid sequence set forth in SEQ ID NO:24.

In some embodiments, the expression vector is a plasmid, phage, virus, or retrovirus.

In a fifth aspect, the disclosure provides a cDNA comprising a polynucleotide encoding a polypeptide comprising: (i) an immunoglobulin heavy chain variable region (VH) comprising VH complementarity determining regions (CDRs) 1, 2, and 3 with the amino acid sequences set forth in SEQ ID NOs:1-3, respectively, wherein the VH when paired with a light chain variable region (VL) comprising the amino acid sequence set forth in SEQ ID NO:8 binds human HSPA5; (ii) an immunoglobulin VL comprising VL CDRs 1, 2, and 3 with the amino acid sequences set forth in SEQ ID NOs:4-6, respectively, wherein the VL when paired with a VH comprising the amino acid sequence set forth in SEQ ID NO:7 binds human HSPA5; (iii) an immunoglobulin VH comprising VH complementarity determining regions (CDRs) 1, 2, and 3 with the amino acid sequences set forth in SEQ ID NOs:11-13, respectively, wherein the VH when paired with a VL comprising the amino acid sequence set forth in SEQ ID NO:18 binds human HSPA5; or (iv) an immunoglobulin VL comprising VL CDRs 1, 2, and 3 with the amino acid sequences set forth in SEQ ID NOs:14-16, respectively, wherein the VL when paired with a VH comprising the amino acid sequence set forth in SEQ ID NO:17 binds human HSPA5.

In a sixth aspect, the disclosure features a host cell comprising: (a) a first expression vector comprising a first polynucleotide encoding a first polypeptide comprising an immunoglobulin heavy chain or a fragment thereof comprising a heavy chain variable region (VH) comprising VH complementarity determining regions (CDRs) 1, 2, and 3 with the amino acid sequences set forth in SEQ ID NOs:1-3, respectively, and a second expression vector comprising a second polynucleotide encoding a second polypeptide comprising an immunoglobulin light chain or a fragment thereof comprising a light chain variable region (VL) comprising VL CDRs 1, 2, and 3 with the amino acid sequences set forth in SEQ ID NOs:4-6, respectively, wherein the immunoglobulin heavy chain or fragment thereof when paired with the immunoglobulin light chain or fragment thereof forms an anti-human HSPA5 antibody or human HSPA5-binding fragment thereof; or (b) a first expression vector comprising a first polynucleotide encoding a first polypeptide comprising an immunoglobulin heavy chain or a fragment thereof comprising a VH comprising VH CDRs 1, 2, and 3 with the amino acid sequences set forth in SEQ ID NOs:11-13, respectively, and a second expression vector comprising a second polynucleotide encoding a second polypeptide comprising an immunoglobulin light chain or a fragment thereof comprising a VL comprising VL CDRs 1, 2, and 3 with the amino acid sequences set forth in SEQ ID NOs:14-16, respectively, wherein the immunoglobulin heavy chain or fragment thereof when paired with the immunoglobulin light chain or fragment thereof forms an anti-human HSPA5 antibody or human HSPA5-binding fragment thereof.

In certain embodiments, the immunoglobulin heavy chain comprises a human IgG1 heavy chain constant region and the immunoglobulin light chain comprises a human lambda light chain constant region. In other embodiments, the immunoglobulin heavy chain comprises a human IgG1 heavy chain constant region and the immunoglobulin light chain comprises a human kappa light chain constant region.

In certain embodiments, the VH consists of the amino acid sequence set forth in SEQ ID NO:7, and the VL consists of the amino acid sequence set forth in SEQ ID NO:8; or the VH consists of the amino acid sequence set forth in SEQ ID NO:17, and the VL consists of the amino acid sequence set forth in SEQ ID NO:18.

In other embodiments, the heavy chain consists of SEQ ID NO:9 and the light chain consists of SEQ ID NO:10; or the heavy chain consists of SEQ ID NO:19 and the light chain consists of SEQ ID NO:20.

In certain embodiments, the host cell is a mammalian host cell. In certain instances, the host cell is a Chinese Hamster Ovary (CHO) cell, a HEK 293 cell, or a NSO cell.

In a seventh aspect, the disclosure features a method of making an antibody or antigen-binding fragment thereof that specifically binds human HSPA5. The method involves culturing the host cell described above in a cell culture and isolating the antibody or antigen-binding fragment from the cell culture.

In certain embodiments, the method further includes formulating the antibody or antigen-binding fragment with a pharmaceutically acceptable carrier into a sterile pharmaceutical composition suitable for administration to a human subject. In some instances, the pharmaceutical composition is suitable for intravenous or subcutaneous administration.

In an eighth aspect, the disclosure provides a pharmaceutical composition comprising an anti-HSPA5 antibody or antigen-binding fragment described above.

In a ninth aspect, the disclosure features a method of delivering an agent to the brain, spinal cord, or other component of the central nervous system of a human subject in need thereof. The method involves administering to the subject the agent and an antibody or antigen-binding fragment thereof that specifically binds human HSPA5.

In certain embodiments, the antibody or antigen-binding fragment and the agent are administered simultaneously. In certain embodiments, the antibody or antigen-binding fragment and the agent are administered sequentially.

In some embodiments, the antibody or antigen-binding fragment that specifically binds human HSPA5 is an antibody or antigen-binding fragment described herein.

In certain embodiments, the agent is an antibody or antigen-binding fragment thereof, a small molecule, siRNA, a degron, or a protein mimotope.

In some embodiments, the agent is an antibody or antigen-binding fragment thereof. Non-limiting examples of such agents include an antibody or antigen-binding fragment that specifically binds tau, an antibody or antigen-binding fragment that specifically binds β-amyloid, an antibody or antigen-binding fragment that specifically binds α-synuclein, an antibody or antigen-binding fragment that specifically binds TDP-43, an antibody or antigen-binding fragment that specifically binds AQP4, an antibody or antigen-binding fragment that specifically binds IL6R, an antibody or antigen-binding fragment that specifically binds CD20, an antibody or antigen-binding fragment that specifically binds CD25, an antibody or antigen-binding fragment that specifically binds VEGF-A, an antibody or antigen-binding fragment that specifically binds BAFF, an antibody or antigen-binding fragment that specifically binds alpha-4-integrin, an antibody or antigen-binding fragment that specifically binds human complement protein C3, an antibody or antigen-binding fragment that specifically binds human complement protein C1q, and an antibody or antigen-binding fragment that specifically binds human complement protein C5.

In certain embodiments, the agent is a small molecule that is a small molecule chemotherapeutic.

In certain embodiments, the agent is a poorly brain-penetrant small molecule.

In a tenth aspect, the disclosure features a method of identifying a human subject at risk of relapse of NMO. The method involves measuring the titer of anti-HSPA5 antibodies in the human subject and determining that the titer of anti-HSPA5 antibodies in the human subject are higher than a control level.

In an eleventh aspect, the disclosure provides a method of determining the severity of an attack of NMO in a human subject. The method involves measuring the titer of anti-HSPA5 antibodies in the human subject and comparing the titer of anti-HSPA5 antibodies in the human subject to a control level.

In a twelfth aspect, the disclosure features a method of diagnosing a human subject with neuropsychiatric systemic lupus erythematosus (NP-SLE). The method involves measuring the titer of anti-HSPA5 antibodies in the human subject and determining that the titer of anti-HSPA5 antibodies in the human subject are higher than a control level.

In a thirteenth aspect, the disclosure provides a method of identifying a human subject at risk of relapse of neuropsychiatric systemic lupus erythematosus (NP-SLE). The method comprises measuring the titer of anti-HSPA5 antibodies in the human subject and determining that the titer of anti-HSPA5 antibodies in the human subject are higher than a control level.

In a fourteenth aspect, the disclosure provides a method of determining the severity of an attack of neuropsychiatric systemic lupus erythematosus (NP-SLE) in a human subject. The method includes measuring the titer of anti-HSPA5 antibodies in the human subject, and comparing the titer of anti-HSPA5 antibodies in the human subject to a control level.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1A provides confocal 3D reconstruction images showing immunostaining for NF-κB p65 (upper panels) and ICAM-1 (lower panels) after exposure to pooled control-IgG or NMO-IgG. Nuclei were counterstained with DAPI. Arrowheads show representative NF-κB p65 nuclear negative cells, and arrows show representative NF-κB p65 positive cells. Three independent experiments were performed for analysis. Scale bar, 50 μm.

FIG. 1B is a bar graph that quantifies nuclear NF-κB p65 positive cells by high content imaging following exposure to pooled IgG from NMO patients or healthy controls. All data are normalized to untreated cultures and shown as mean±SEM from 3 experiments in triplicate. P-values were assessed with one-way ANOVA followed by Tukey's or Dunnett's multiple comparisons test (***<0.001).

FIG. 1C is a bar graph that quantifies the effect of IgG from patients (NMO or SLE) and healthy controls on NF-κB p65 nuclear translocation in BMECs. IgG from patients NMO 1 and NMO 2 as well as patients SLE 2, 3, and 4 show significantly greater NF-κB p65 nuclear translocation than control IgG (***P<0.001; **P<0.01; *P<0.05 vs control, One-way ANOVA). Error bars indicate SEM (n=4 in triplicate).

FIG. 1D provides a table summarizing the effect of serum IgG from NMO patients, SLE patients, and healthy controls on the activation of BMECs.

FIG. 2 is a table summarizing the effect of pooled IgG and 14 rAbs from NMO patients and controls on the activation of BMECs.

FIG. 3A shows confocal 3D reconstruction images showing immunostaining for NF-κB p65 (upper panels) and ICAM-1 (lower panels) after exposure to control-rAb or NMO-rAb ON-12-2-46. Nuclei were counterstained with DAPI. Arrowheads show representative NF-κB p65 nuclear negative cells, and arrows show representative NF-κB p65 positive cells. Three independent experiments were performed for analysis. Scale bar, 50 μm.

FIG. 3B is a bar graph that quantifies nuclear NF-κB p65 positive cells by high content imaging following exposure to NMO-rAb ON-12-2-46 or 2 isotype control rAbs. All data are normalized to untreated cultures and shown as mean±SEM from 3 experiments in triplicate. P-values were assessed with one-way ANOVA followed by Tukey's multiple comparisons test (***<0.001).

FIG. 3C is a bar graph that quantifies nuclear NF-κB p65 positive BMECs by high content imaging after exposure to pooled NMO-IgG and pooled control-IgG. Data are normalized to untreated cultures and presented as mean±SEM from 3 experiments in triplicate. Statistical significance was assessed with one-way ANOVA followed by Dunnett's multiple comparisons test (***P<0.001; **P<0.01; *P<0.05 vs NMO-IgG 12.5).

FIG. 3D is a bar graph that quantifies nuclear NF-κB p65 positive BMECs by high content imaging after exposure to NMO-rAb ON-12-2-46 and control-rAb. Data are normalized to untreated cultures and presented as mean±SEM from 3 experiments in triplicate. Statistical significance was assessed with one-way ANOVA followed by Dunnett's multiple comparisons test (***P<0.001; **P<0.01; *P<0.05 vs NMO-rAb 3.125).

FIG. 3E is a bar graph that shows a dose-dependent effect of pooled NMO-IgG on ICAM-1 up-regulation in BMECs. Addition of both TNF-α and IFN-β was used as positive control and pooled control-IgG or control-rAb was served as negative control. Data normalized to untreated culture are mean±SEM from 2 independent experiments with triplicate.

FIG. 3F is a bar graph that shows a dose-dependent effect of NMO-rAb (ON-12-2-46) on ICAM-1 up-regulation in BMECs. Addition of both TNF-α and IFN-β was used as positive control and pooled control-IgG or control-rAb was served as negative control. Data normalized to untreated culture are mean±SEM from 2 independent experiments with triplicate.

FIG. 4A provides confocal images of claudin-5 and DAPI labeling in BMECs after incubation with pooled control-IgG (400 μg/mL), pooled NMO-IgG (400 μg/mL), control-rAb (50 μg/mL), or NMO-rAb ON-12-2-46 (50 μg/mL). Scale bar, 50 μm.

FIG. 4B is a bar graph depicting the claudin-5 area fraction determined from the confocal micrograph images. Data are mean±SEM with n=3 experiments in triplicate. P-values were obtained with one-way ANOVA followed by Sidak's multiple comparisons test (***P<0.001).

FIG. 4C is a bar graph illustrating the effect of NMO-rAb ON-12-2-46 on the permeability of BMECs to 10-kDa dextran. Data are mean±SEM from n=3 experiments in triplicate. P-value were assayed by one-way ANOVA followed by Tukey's multiple comparisons test (*P<0.05).

FIG. 4D is a bar graph showing the effect of NMO-rAb ON-12-2-46 on the permeability of BMECs to IgG. Data are mean±SEM with n=3 independent experiments. P-value were obtained by Mann-Whitney U test (***P<0.001).

FIG. 5A shows the results of polyacrylamide gel electrophoresis (PAGE) and immunoblot analysis of crude membrane protein lysates prepared from U87MG and OL cells. Multiple protein bands reacted with the respective NMO-rAbs. The unlabeled lanes contain the molecular weight standards.

FIG. 5B is a cartoon detailing the scheme that was used to purify the antigenic target of rAbs ON-12-2-46 and ON-07-5-31. X-linker, cross-linker.

FIG. 5C shows the results of PAGE and immunoblot analysis of protein eluted from the DTSSP cross-linked-rAb with DTT. Lane 1, molecular weight standard; Lane 2, protein bound to ON-07-5-31 rAb; Lane 3, protein bound to ON-12-2-46 rAb). The blots were probed with the indicated NMO-rAb. Common protein band recovered for mass spectrometry is indicated by arrow.

FIG. 5D shows the results of double immunofluorescence labeling of U87MG cells with ON-07-5-31 rAb and rabbit anti-GRP78 antisera and shows co-localization of the two proteins (merge).

FIG. 5E shows U87MG cells treated for 24 hours with either DMSO (control) or 0.5 mM thapsigargin, a known inducer of GRP78 (showed increased expression when double-labeled with ON-12-2-46 rAb and rabbit anti-GRP78 antisera). Co-localization of the two proteins is shown in the merge.

FIG. 5F is an immunoblot analysis of commercial recombinant GRP78 protein purified from HEK cells (lane 1) or bacteria (lane 2) and probed with the rAb indicated or rabbit anti-GRP78 sera. This analysis demonstrated that the rAb recognized GRP78.

FIG. 5G shows competition immunofluorescence assays using commercial recombinant GRP78 protein (5 μg) to block rAb binding (2 μg) to U87MG cells. These images show decreased binding of ON-12-2-46 to the U87MG cells. The nuclei are stained by DAPI.

FIG. 5H is a schematic demonstrating the organization of endothelial cells, astrocytes, and pericytes in brain microvasculature.

FIG. 5I shows 3D reconstruction confocal images of non-permeabilized mouse brain tissue stained with NMO-rAb ON-12-2-46, AQP4, and claudin-5. GRP78 staining co-localizes with claudin-5 on endothelial cells internal to AQP4.

FIG. 6 is a bar graph comparing the number of cells positive for nuclear NF-κB p65 between pooled NMO-IgG immunoadsorbed against SNAP25 or GRP78. All data are shown as mean±SEM from 5 experiments in triplicate. Statistical significance was assessed by paired t-test (*P<0.05).

FIG. 7 is a bar graph depicting the results of high content imaging and quantification of nuclear NF-κB p65 positive BMECs normalized to untreated culture after exposure to 2 commercial anti-GRP78 antibodies (GRP78 Abl, ab12223; GRP78 Ab2, sc-1051) and 2 control-IgG (control-IgG 1, rabbit-IgG; control-IgG 2, goat-IgG). Data are mean±SEM from 3 experiments in triplicate. Statistical significance was assessed with one-way ANOVA (***P<0.001 vs control-IgG1 80 μg/ml or control-IgG2 80 μg/ml).

FIG. 8A shows 3D confocal images of mouse coronal brain sections following administration of murinized GRP78-specific NMO-rAb ON-12-2-46 or control rAb in combination with human AQP4-specific rAb ON-7-5-53, immunostained for human IgG and AQP4. Local extravasation of human IgG into brain extracellular space is noted only in mice treated with GRP78-specific NMO-rAb ON-12-2-46.

FIG. 8B shows 3D confocal images of mouse coronal brain sections immunostained for human IgG (rAb ON-7-5-53) and mouse fibrinogen following administration of murinized GRP78-specific NMO rAb ON-12-2-46 or control rAb in combination with human AQP4-specific rAb ON-7-5-53. Local extravasation of human IgG and fibrinogen into brain extracellular space is noted only in mice treated with GRP78-specific NMO-rAb ON-12-2-46.

FIG. 8C provides 3D confocal images of mouse coronal brain sections immunostained for human IgG, mouse albumin, and AQP4 following administration of murinized GRP78-specific NMO-rAb ON-12-2-46 or control rAb in combination with human AQP4-specific rAb ON-7-5-53. Local extravasation of human IgG and albumin into brain extracellular space is noted only in mice treated with GRP78-specific NMO-rAb ON-12-2-46.

FIG. 8D is a bar graph depicting the average diameter of vessels (mean±SEM) in mice treated with GRP78-specific NMO-rAb ON-12-2-46 and control rAb. Vessels were quantified from 35 (control rAb-injected) and 31 (ON-12-2-46-injected) 20× high-power fields and the diameters measured using Axio Vision LE. P-values were calculated by Mann Whitney U test (***P<0.0001).

### DETAILED DESCRIPTION

The presence of aquaporin-4 (AQP4) antibodies alone in sera is insufficient to initiate neuromyelitis optica (NMO) disease attacks. Thus, blood-brain barrier (BBB) breakdown and subsequent diffusion of astrocyte-specific AQP4-IgG into the central nervous system (CNS) appears to be critical for NMO pathogenesis. Applicants have demonstrated that a non-AQP4 specific antibody from NMO patients activates brain microvascular endothelial cells (BMECs) via canonical NF-κB signaling and results in increased macromolecular permeability accompanied by decreased claudin-5 expression. Using proteomic analysis, HSPA5/GRP78 was identified as the antigenic target of non-AQP4 specific antibodies from NMO patients. Moreover, depleting GRP78 antibodies from NMO-IgG by immunoadsorption significantly and substantially decreased NF-κB translocation in treated BMECs. These findings suggest that HSPA5/GRP78 autoantibodies contribute to BBB breakdown and NMO attacks in some patients. Taken together, these findings indicate that targeting HSPA5 may facilitate or enhance delivery of an agent of interest (e.g., a therapeutic antibody, small molecule drug) to the brain, spinal cord, or other component of the CNS. In addition, HSPA5 can serve as a biomarker of incipient disease activity in NMO and neuropsychiatric systemic lupus erythematosus (NP-SLE).

This disclosure provides anti-HSPA5 antibodies. These antibodies can have one or more of these properties: (i) ability to specifically bind HSPA5 (e.g., human HSPA5); (ii) ability to upregulate ICAM-1 expression on BMECs; (iii) ability to induce nuclear translocation of NF-κB in BMECs; (iv) ability to specifically bind BMECs relative to other microvascular endothelial cells (MECs), e.g., HUVECs, MECS from kidney or dermis; (v) ability to induce a structural change of tight junctions; and (vi) ability to enhance BBB permeability in vivo. In some embodiments, the anti-HSPA5 antibodies of this disclosure have two, three, four, five, or all six of the above-listed properties. This disclosure also features a platform technology that provides a way of introducing an agent of interest to a component of the central nervous system. Specifically, methods of using anti-HSPA5 antibodies to introduce an agent of interest to the brain, spinal cord, or other component of the CNS are disclosed. Furthermore, methods of determining whether a human subject is likely to exhibit a relapse of NMO or NP-SLE; methods of diagnosing NP-SLE; and methods of determining the severity of an attack of NMO or NP-SLE are also disclosed herein.

### HSPA5

Heat Shock Protein Family A (Hsp70) Member 5 (HSPA5) also known as Binding immunoglobulin protein (BiP), 78 kDa glucose-regulated protein (GRP78), MIF2, and FLJ26106 is a Hsp70-type molecular chaperone of the endoplasmic reticulum where it promotes the folding, maturation, and assembly of nascent proteins and also coordinates the unfolded protein response (Lee A S, Nat Rev Cancer, 14:263-76 (2014)). HSPA5 is a constitutively expressed protein, but its levels increase substantially in response to challenging conditions such as hypoxia and nutrient deprivation. In fact, HSPA5 has been implicated in cytoprotection and chemoresistance in the tumor microenvironment where hypoxic conditions prevail. HSPA5 knockout mice have an early embryonic lethal phenotype; HSPA5 was found to be required for proliferation and survival of embryonic inner cell mass cells that are the precursors of pluripotent stem cells.

In addition to its role as an ER localized chaperone, HSPA5 can be translocated to other cellular locations, including the cytosol, mitochondria, nucleus, and cell membrane (Ni M et al, Biochem J., 434:181-8 (2011); Gray et al., FEBS Letts., 586:1836-1845 (2012)). Expressed on the cell surface, HSPA5 is involved in signal transduction. Through complexing with partners such as HTJ-1, HSPA5 participates in recognition of extracellular ligands and regulates proliferation and viability via the activation of PI3K/AKt or NF-κB p65 signaling pathways (Misra U K et al., J. Biol. Chem., 281: 13694-707 (2006)).

The amino acid sequence of the human HSPA5 protein (Genbank® Accession No. P11021) is shown below:

(SEQ ID NO: 29)

| | |
|---|---|
| 1 | MKLSLVAAML LLLSAARAEE EDKKEDVGTV VGIDLGTTYS |
| | CVGVFKNGRV EIIANDQGNR |
| 61 | ITPSYVAFTP EGERLIGDAA KNQLTSNPEN TVFDAKRLIG |
| | RTWNDPSVQQ DIKFLPFKVV |
| 121 | EKKTKPYIQV DIGGGQTKTF APEEISAMVL TKMKETAEAY |
| | LGKKVTHAVV TVPAYFNDAQ |
| 181 | RQATKDAGTI AGLNVMRIIN EPTAAAIAYG LDKREGEKNI |
| | LVFDLGGGTF DVSLLTIDNG |
| 241 | VFEVVATNGD THLGGEDFDQ RVMEHFIKLY KKKTGKDVRK |
| | DNRAVQKLRR EVEKAKRALS |
| 301 | SQHQARIEIE SFYEGEDFSE TLTRAKFEEL NMDLFRSTMK |
| | PVQKVLEDSD LKKSDIDEIV |
| 361 | LVGGSTRIPK IQQLVKEFFN GKEPSRGINP DEAVAYGAAV |
| | QAGVLSGDQD TGDLVLLDVC |
| 421 | PLTLGIETVG GVMTKLIPRN TVVPTKKSQI FSTASDNQPT |
| | VTIKVYEGER PLTKDNHLLG |
| 481 | TFDLTGIPPA PRGVPQIEVT FEIDVNGILR VTAEDKGTGN |
| | KNKITITNDQ NRLTPEEIER |
| 541 | MVNDAEKFAE EDKKLKERID TRNELESYAY SLKNQIGDKE |
| | KLGGKLSSED KETMEKAVEE |
| 601 | KIEWLESHQD ADIEDFKAKK KELEEIVQPI ISKLYGSAGP |
| | PPTGEEDTAE KDEL |

The signal sequence MKLSLVAAMLLLLSAARA (SEQ ID NO:30) is underlined; the mature human HSPA5 protein is amino acids 19-654 of SEQ ID NO:29; the nucleotide binding domain is amino acids 125-280 of SEQ ID NO:29; and the substrate binding domain (also known as the peptide binding domain) is amino acids 400-500 of SEQ ID NO:29 (see, e.g., A. S. Lee, Nat. Rev. Cancer, 14(4): 263-276 (2014)).

### Anti-HSPA5 Antibodies

The antibodies or antigen-binding fragment thereof of this disclosure specifically bind to HSPA5. In specific embodiments, these antibodies or antigen-binding fragments specifically bind to human HSPA5. “Specifically binds” as used herein means that the antibody or antigen-binding fragment preferentially binds HSPA5 (e.g., human HSPA5, mouse HSPA5) over other proteins. In certain instances, the anti-HSPA5 antibodies of the disclosure have a higher affinity for HSPA5 than for other proteins. Anti-HSPA5 antibodies that specifically bind HSPA5 may have a binding affinity for human HSPA5 of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. Methods of measuring the binding affinity of an antibody are well known in the art and include Surface Plasmon Resonance (SPR) (Morton and Myszka “Kinetic analysis of macromolecular interactions using surface plasmon resonance biosensors” Methods in Enzymology (1998) 295, 268-294), Bio-Layer Interferometry, (Abdiche et al “Determining Kinetics and Affinities of Protein Interactions Using a Parallel Real-time Label-free Biosensor, the Octet” Analytical Biochemistry (2008) 377, 209-217), Kinetic Exclusion Assay (KinExA) (Darling and Brault “Kinetic exclusion assay technology: characterization of molecular interactions” Assay and Drug Dev Tech (2004) 2, 647-657), isothermal calorimetry (Pierce et al “Isothermal Titration calorimetry of Protein-Protein Interactions” Methods (1999) 19, 213-221) and analytical ultracentrifugation (Lebowitz et al “Modern analytical ultracentrifugation in protein science: A tutorial review” Protein Science (2002), 11:2067-2079).

The anti-HSPA5 antibodies of this disclosure have one or more of the following characteristics: (i) ability to specifically bind HSPA5 (e.g., human HSPA5 and/or mouse HSPA5); (ii) ability to upregulate ICAM-1 expression on BMECs; (iii) ability to induce nuclear translocation of NF-κB or activation of other stimulus sensitive transcriptional regulatory mechanisms in BMECs; (iv) ability to specifically bind BMECs relative to other microvascular endothelial cells (MECs), e.g., HUVECs, MECS from kidney or dermis; (v) ability to induce a structural change of tight junctions; and (vi) ability to enhance BBB permeability. In certain instances, the anti-HSPA5 antibody is not one of the commercial anti-HSPA5 antibodies (e.g., Santa Cruz, sc-376768, sc-1051, sc-1050, sc-13968; Abcam, ab21685, ab12223; Sigma G8918).

It is to be understood that when reference is made to anti-HSPA5 antibodies herein, that it encompasses not only whole antibodies but also HSPA5-binding antibody fragments, minibodies, nanobodies, VHHs, and VNARs.

### Antibody Fragments

The present disclosure encompasses the antibody fragments or domains described herein that retains the ability to specifically bind to HSPA5 (e.g., human HSPA5). Antibody fragments include, e.g., Fab, Fab′, F(ab′)₂, Facb, and Fv. These fragments may be humanized or fully human. Antibody fragments may be prepared by proteolytic digestion of intact antibodies. For example, antibody fragments can be obtained by treating the whole antibody with an enzyme such as papain, pepsin, or plasmin. Papain digestion of whole antibodies produces F(ab)₂ or Fab fragments; pepsin digestion of whole antibodies yields F(ab′)2 or Fab′; and plasmin digestion of whole antibodies yields Facb fragments. Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

### Minibodies

Also encompassed are minibodies of the antibodies described herein. Minibodies of anti-HSPA5 antibodies include diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A., 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. U.S.A., 85:5879-5883 (1988); and Pluckthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-HSPA5 antibody or antigen-binding fragment thereof described herein.

An sc(Fv)2 is a minibody in which two VHs and two VLs are linked by a linker to form a single chain (Hudson, et al., J. Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however, the order of the two VHs and two VLs is not limited to the above arrangement, and they may be arranged in any order. Examples of arrangements are listed below:

[VL] linker [VH] linker [VH] linker [VL]

[VH] linker [VL] linker [VL] linker [VH]

[VH] linker [VH] linker [VL] linker [VL]

[VL] linker [VL] linker [VH] linker [VH]

[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about three to 25 residues (e.g., 5, 6, 7, 8, 9, 10, 11, 12, 13, 14, 15, 16, 17, 18) can be used as a linker. Examples of such peptide linkers include: Ser; Gly Ser; Gly Gly Ser; Ser Gly Gly; Gly Gly Gly Ser (SEQ ID NO:33); Ser Gly Gly Gly (SEQ ID NO:34); Gly Gly Gly Gly Ser (SEQ ID NO:35); Ser Gly Gly Gly Gly (SEQ ID NO:36); Gly Gly Gly Gly Gly Ser (SEQ ID NO:37); Ser Gly Gly Gly Gly Gly (SEQ ID NO:38); Gly Gly Gly Gly Gly Gly Ser (SEQ ID NO:39); Ser Gly Gly Gly Gly Gly Gly (SEQ ID NO:40); (Gly Gly Gly Gly Ser (SEQ ID NO:35)ₙ, wherein n is an integer of one or more; and (Ser Gly Gly Gly Gly (SEQ ID NO:36)ₙ, wherein n is an integer of one or more. In certain embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the antibody fragments or minibodies may include modifications such as substitutions, deletions, additions, and/or insertions. For example, the modification may be in one or more of the CDRs of the anti-HSPA5 antibody (e.g., Exemplary Anti-human HSPA5 Antibody 1 or Exemplary Anti-human HSPA5 Antibody 2). In certain embodiments, the modification involves one, two, or three amino acid substitutions in one, two, or three CDRs of the VH and/or one, two, or three CDRs of the VL domain of the anti-HSPA5 minibody. Such substitutions are made to improve the binding and/or functional activity of the anti-HSPA5 minibody. In other embodiments, one, two, or three amino acids of one or more of the six CDRs of the anti-HSPA5 antibody or antigen-binding fragment thereof may be deleted or added as long as there is HSPA5 binding and/or functional activity when VH and VL are associated.

### VHH

VHH also known as nanobodies are derived from the antigen-binding variable heavy chain regions (VHHs) of heavy chain antibodies found in camels and llamas, which lack light chains. The present disclosure encompasses VHHs that specifically bind HSPA5.

### Variable Domain of New Antigen Receptors (VNARs)

A VNAR is a variable domain of a new antigen receptor (IgNAR). IgNARs exist in the sera of sharks as a covalently linked heavy chain homodimer. It exists as a soluble and receptor bound form consisting of a variable domain (VNAR) with differing numbers of constant domains. The VNAR is composed of a CDR1 and CDR3 and in lieu of a CDR2 has HV2 and HV4 domains (see, e.g., Barelle and Porter, Antibodies, 4:240-258 (2015)). The present disclosure encompasses VNARs that specifically bind HSPA5.

### Constant Regions

Antibodies of this disclosure can be whole antibodies or single chain Fc (scFc) and can comprise any constant region known in the art. The light chain constant region can be, for example, a kappa- or lambda-type light chain constant region, e.g., a human kappa or human lambda light chain constant region. The heavy chain constant region can be, e.g., an alpha-, delta-, epsilon-, gamma-, or mu-type heavy chain constant region, e.g., a human alpha-, human delta-, human epsilon-, human gamma-, or human mu-type heavy chain constant region. In certain instances, the anti-HSPA5 antibody is an IgA antibody, an IgD antibody, an IgE antibody, an IgG1 antibody, an IgG2 antibody, an IgG3 antibody, an IgG4 antibody, or an IgM antibody.

In one embodiment, the light or heavy chain constant region is a fragment, derivative, variant, or mutein of a naturally occurring constant region. In some embodiments, the variable heavy chain of the anti-HSPA5 antibodies described herein is linked to a heavy chain constant region comprising a CH1 domain and a hinge region. In some embodiments, the variable heavy chain is linked to a heavy chain constant region comprising a CH2 domain. In some embodiments, the variable heavy chain is linked to a heavy chain constant region comprising a CH3 domain. In some embodiments, the variable heavy chain is linked to a heavy chain constant region comprising a CH2 and CH3 domain. In some embodiments, the variable heavy chain is linked to a heavy chain constant region comprising a hinge region, a CH2 and a CH3 domain. The CH1, hinge region, CH2, and/or CH3 can be from an IgG antibody (e.g., IgG1, IgG4). In certain embodiments, the variable heavy chain of an anti-HSPA5 antibody described herein is linked to a heavy chain constant region comprising a CH1 domain, hinge region, and CH2 domain from IgG4 and a CH3 domain from IgG1. In certain embodiments such a chimeric antibody may contain one or more additional mutations in the heavy chain constant region that increase the stability of the chimeric antibody. In certain embodiments, the heavy chain constant region includes substitutions that modify the properties of the antibody (e.g., decrease Fc receptor binding, increase or decrease antibody glycosylation, decrease binding to C1q).

In certain embodiments, an anti-HSPA5 antibody of this disclosure is an IgG isotype antibody. In one embodiment, the antibody is IgG1. In another embodiment, the antibody is IgG2. In yet another embodiment, the antibody is IgG4. In some instances, the IgG4 antibody has one or more mutations that reduce or prevent it adopting a functionally monovalent format. For example, the hinge region of IgG4 can be mutated to make it identical in amino acid sequence to the hinge region of human IgG1 (mutation of a serine in human IgG4 hinge to a proline). In some embodiments, the antibody has a chimeric heavy chain constant region (e.g., having the CH1, hinge, and CH2 regions of IgG4 and CH3 region of IgG1). In certain embodiments, the antibody includes a human Fc region that binds human CD16a, human CD32a, human CD32b, and human CD64 with a reduced binding affinity as compared to a wild type IgG1 antibody (e.g., Exemplary Antibody 1 or Exemplary Antibody 2).

### Bispecific Antibodies

In certain embodiments, an anti-HSPA5 antibody of this disclosure is a bispecific antibody. Bispecific antibodies are antibodies that have binding specificities for at least two different epitopes. Exemplary bispecific antibodies may bind to two different epitopes of the HSPA5 protein. Other such antibodies may combine a HSPA5 binding site with a binding site for another protein (e.g., tau, β-amyloid, α-synuclein, TDP-43, AQP4, IL6R, CD20, CD25, VEGF-A, BAFF, alpha-4-integrin, human complement protein C3, human complement protein C1q, human complement protein C5). Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Conjugated Antibodies

The antibodies or antigen-binding fragments disclosed herein may be conjugated to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), human serum albumin or a fragment thereof, radioactive materials (e.g., ⁹⁰Y, ¹³¹I), fluorescent substances, luminescent substances, haptens, enzymes, metal chelates, and drugs.

In certain embodiments, an anti-HSPA5 antibody or antigen-binding fragment thereof are modified with a moiety that improves its stabilization and/or retention in circulation, e.g., in blood, serum, or other tissues, e.g., by at least 1.5, 2, 5, 10, 15, 20, 25, 30, 40, or 50 fold. For example, the anti-HSPA5 antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-HSPA5 antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

In certain embodiments, when the anti-HSPA5 antibodies disclosed herein are being used to ferry another antibody across the BBB, the other antibody or antigen-binding fragment thereof may be coupled to a cytotoxic agent (e.g., chemotherapeutic drugs, bacterial toxins or plant toxins (immunotoxins), radiopharmaceutical agents) (see, e.g., Thomas et al., Lancet Oncol., 17:e254-62 (2016) incorporated by reference herein in its entirety). For example, the antibody or antigen-binding fragment may be one that is targeted to tumor cells in a component of the central nervous system (e.g., brain, spinal cord). In certain embodiments, the cytotoxic agent is a radioisotope (e.g., Y-90, I-131, Bi-213, Ga-67, At-211, Pb-212, In-111, Lu-177, Re-188) (see, e.g., Kawashima, The Scientific World Journal, vol. 2014, Article ID 492061, 10 pages, (2014) incorporated by reference herein in its entirety). In certain embodiments, the cytotoxic agent is selected from the group consisting of SN-38, doxorubicin, a vinca alkaloid (e.g., vinblastine), methotrexate, calicheamicin, a maytasine derivative (DM1/emtansine, DM4/ravtansine), auristatin (MMAE, MMAF), and pyrrolobenzodiazepine. In certain embodiments, the antibody or antigen-binding fragment is directed to CD20, CD22, CD33, CD74, CEA, A33 glycoprotein, CD138, HER-2, Na-dependent phosphate transporter, MUC-1, HLA-DR, Ep-CAM, TAG-72, EGFR, or Tenascin. Such antibody-cytotoxic conjugates are useful to kill tumor cells, e.g., where a tumor metastasizes to brain (e.g., NHL, breast cancer, melanoma).

The above-described conjugated antibodies or fragments can be prepared by performing chemical modifications on the antibodies or the lower molecular weight forms thereof described herein. Methods for modifying antibodies are well known in the art (e.g., U.S. Pat. Nos. 5,057,313 and 5,156,840).

This disclosure provides two exemplary anti-HSPA5 antibodies that are described in more detail below.

### Exemplary Anti-Human HSPA5 Antibody 1

The amino acid sequences of the six Complementarity-determining regions (CDRs) 1, 2, and 3, according to enhanced Chothia (www.bioinf.org.uk/abs/), of Exemplary Anti-human HSPA5 Antibody 1 are shown below.

VH CDR1
(SEQ ID NO: 1)
GYWWT
VH CDR2
(SEQ ID NO: 2)
EINPSDTTNYNPSLKS
VH CDR3
(SEQ ID NO: 3)
ARGRSTYGASTFYNYYYFDS
VL CDR1
(SEQ ID NO: 4)
RASQKIDRWLA
VL CDR2
(SEQ ID NO: 5)
KASNLES
VL CDR3
(SEQ ID NO: 6)
QQYQNYPYT

The amino acid sequence of the mature Exemplary Anti-human HSPA5 Antibody 1 heavy chain variable region (VH) is shown below (the three CDRs are underlined).

(SEQ ID NO: 7)
QVQLQQWGAGLLKPSETLSLTCAVYGGSFSGYWWTWIRQSPEKGLEWIGEI
NPSDTTNYNPSLKSRVAISKDSSKNQFSLRLSSVTAADTALYFCARGRSTY
GASTFYNYYYFDSWGQGSRVVVSS

In some instances, the VH of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:7. In some instances, the VH of an anti-human HSPA5 antibody of this disclosure comprises or consists of an amino acid sequence that is identical to SEQ ID NO:7 except for 1, 2, 3, 4, 5, 6, 7, 8, 9, or 10 amino acid substitutions, deletions, and/or insertions. These amino acid substitutions, deletions, and/or insertions may be introduced to, e.g., increase the affinity, stability of the antibody or antigen-binding fragment thereof. In particular embodiments, the VH comprises one, two, or three VH CDRs (VH CDR1, VH CDR2, and VH CDR3) according to any CDR definition (e.g., Kabat, Chothia, enhanced Chothia, AbM, or contact definitions) of the VH having the amino acid sequence set forth in SEQ ID NO:7. Alternate CDR definitions can be determined, e.g., by using the AbYsis database (www.bioinf.org.uk/abysis/sequence_input/key_annotation/key_annotation.cgi). In certain instances, the VH of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence MEWSWVFLFFLSVTTGVHS (SEQ ID NO:31).

The amino acid sequence of the mature Exemplary Anti-human HSPA5 Antibody 1 light chain variable region (VL) is shown below (CDRs are underlined).

(SEQ ID NO: 8)
DIQMTQSPSTLSASVGDTITITCRASQKIDRWLAWYQQKPGKAPKLLIYKA
SNLESGVPPRLSGTGSGTDFTLTIKSLQPDDFATYFCQQYQNYPYTFGPGT
KLEMRR

In some instances, the VL of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:8. In some instances, the VL of an anti-human HSPA5 antibody of this disclosure comprises or consists of an amino acid sequence that is identical to SEQ ID NO:8 except for 1, 2, 3, 4, 5, 6, 7, 8, 9, or 10 amino acid substitutions, deletions, and/or insertions. These amino acid substitutions, deletions, and/or insertions may be introduced to, e.g., increase the affinity, stability of the antibody or antigen-binding fragment thereof. In particular embodiments, the VL comprises one, two, or three VL CDRs (VL CDR1, VL CDR2, and VL CDR3) according to any CDR definition (e.g., Kabat, Chothia, enhanced Chothia, AbM, or contact definitions) of the VL having the amino acid sequence set forth in SEQ ID NO:8. In certain instances, the VL of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence MGVPTQVLGLLLLWLTDARC (SEQ ID NO:32).

The amino acid sequence of the mature Exemplary Anti-human HSPA5 Antibody 1 heavy chain is shown below (the three CDRs are underlined; the heavy chain constant region (IgG1) is boldened).

(SEQ ID NO: 9)
QVQLQQWGAGLLKPSETLSLTCAVYGGSFSGYWWTWIRQSPEKGLEWIGEI
NPSDTTNYNPSLKSRVAISKDSSKNQFSLRLSSVTAADTALYFCARGRSTY
GASTFYNYYYFDSWGQGSRVVVSSASTKGPSVFPLAPSSKSTSGGTAALGC
LVKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEEYN
STYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK

In some instances, the heavy chain of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:9. In some instances, the heavy chain of an anti-human HSPA5 antibody of this disclosure comprises or consists of an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the heavy chain constant region of the amino acid sequence set forth in SEQ ID NO:9. In some instances, the heavy chain of an anti-human HSPA5 antibody of this disclosure comprises or consists of an amino acid sequence that is identical to SEQ ID NO:9 except for 1, 2, 3, 4, 5, 6, 7, 8, 9, 10, 11, 12, 13, 14, 15, 16, 17, 18, 19, or 20 amino acid substitutions, deletions, and/or insertions. These amino acid substitutions, deletions, and/or insertions may be introduced to, e.g., increase the affinity, stability of the antibody or antigen-binding fragment thereof. In some instances, the heavy chain of an anti-human HSPA5 antibody of this disclosure comprises or consists of an amino acid sequence that is identical to SEQ ID NO:9 except for 1, 2, 3, 4, 5, 6, 7, 8, 9, 10, 11, 12, 13, 14, 15, 16, 17, 18, 19, or 20 amino acid substitutions, deletions, and/or insertions in the heavy chain constant region of the amino acid sequence set forth in SEQ ID NO:9. In particular embodiments of the above instances, the heavy chain comprises one, two, or three VH CDRs (VH CDR1, VH CDR2, and VH CDR3) according to any CDR definition (e.g., Kabat, Chothia, enhanced Chothia, AbM, or contact definitions) of the VH having the amino acid sequence set forth in SEQ ID NO:7. In certain embodiments of the above instances, the heavy chain of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence

(SEQ ID NO: 31)
MEWSWVFLFFLSVTTGVHS

The amino acid sequence of the mature Exemplary Anti-human HSPA5 Antibody 1 light chain is shown below (the three CDRs are underlined; the light chain constant region (Vκ) is boldened).

(SEQ ID NO: 10)
DIQMTQSPSTLSASVGDTITITCRASQKIDRWLAWYQQKPGKAPKLLIYKA
SNLESGVPPRLSGTGSGTDFTLTIKSLQPDDFATYFCQQYQNYPYTFGPGT
KLEMRRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

In some instances, the light chain of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:10. In some instances, the light chain of an anti-human HSPA5 antibody of this disclosure comprises or consists of an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the heavy chain constant region of the amino acid sequence set forth in SEQ ID NO:10. In some instances, the light chain of an anti-human HSPA5 antibody of this disclosure comprises or consists of an amino acid sequence that is identical to SEQ ID NO:10 except for 1, 2, 3, 4, 5, 6, 7, 8, 9, 10, 11, 12, 13, 14, 15, 16, 17, 18, 19, or 20 amino acid substitutions, deletions, and/or insertions. These amino acid substitutions, deletions, and/or insertions may be introduced to, e.g., increase the affinity, stability of the antibody or antigen-binding fragment thereof. In some instances, the heavy chain of an anti-human HSPA5 antibody of this disclosure comprises or consists of an amino acid sequence that is identical to SEQ ID NO:10 except for 1, 2, 3, 4, 5, 6, 7, 8, 9, 10, 11, 12, 13, 14, 15, 16, 17, 18, 19, or 20 amino acid substitutions, deletions, and/or insertions in the light chain constant region of the amino acid sequence set forth in SEQ ID NO:10. In particular embodiments of the above instances, the light chain comprises one, two, or three VL CDRs (VL CDR1, VL CDR2, and VL CDR3) according to any CDR definition (e.g., Kabat, Chothia, enhanced Chothia, AbM, or contact definitions) of the VL having the amino acid sequence set forth in SEQ ID NO:8. In certain embodiments of the above instances, the light chain of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence

(SEQ ID NO: 32)
MGVPTQVLGLLLLWLTDARC

In certain instances, the anti-HSPA5 antibody is not one of the commercial anti-HSPA5 antibodies (e.g., Santa Cruz, sc-376768, sc-1051, Abcam GRP78 Abl, ab12223).

Exemplary anti-HSPA5 Antibody 1 is an IgG1/kappa antibody with a heavy chain of SEQ ID NO:9 and a light chain of SEQ ID NO:10.

### Exemplary Anti-Human HSPA5 Antibody 2

The amino acid sequences of the six Complementarity-determining regions (CDRs) 1, 2, and 3, according to enhanced Chothia (www.bioinf.org.uk/abs/), of Exemplary Anti-human HSPA5 Antibody 2 are shown below.

VH CDR1
(SEQ ID NO: 11)
GSGIH
VH CDR2
(SEQ ID NO: 12)
HIRSKSDGSATLYAASVRG
VH CDR3
(SEQ ID NO: 13)
ARYRRVAVAGYTYYYYMDV
VL CDR1
(SEQ ID NO: 14)
RASQSIDSYLN
VL CDR2
(SEQ ID NO: 15)
AASSLQS
VL CDR3
(SEQ ID NO: 16)
QQSHSLPRT

The amino acid sequence of the mature Exemplary Anti-human HSPA5 Antibody 2 heavy chain variable region (VH) is shown below (the three CDRs are boldened).

(SEQ ID NO: 17)
EVQLVESGGGLVHPGESLRLSCAASGFSLSGSGIHWVRQASGKGLEWLGHI
RSKSDGSATLYAASVRGRFIISRDDSTNTAYLHMSSLRNEDTAVYYCARYR
RVAVAGYTYYYYMDVWGKGSTVSVSS

In some instances, the VH of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:17. In some instances, the VH of an anti-human HSPA5 antibody of this disclosure comprises or consists of an amino acid sequence that is identical to SEQ ID NO:17 except for 1, 2, 3, 4, 5, 6, 7, 8, 9, or 10 amino acid substitutions, deletions, and/or insertions. These amino acid substitutions, deletions, and/or insertions may be introduced to, e.g., increase the affinity, stability of the antibody or antigen-binding fragment thereof. In particular embodiments, the VH comprises one, two, or three VH CDRs (VH CDR1, VH CDR2, and VH CDR3) according to any CDR definition (e.g., Kabat, Chothia, enhanced Chothia, AbM, or contact definitions) of the VH having the amino acid sequence set forth in SEQ ID NO:17. In certain instances, the VH of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence MEWSWVFLFFLSVTTGVHS (SEQ ID NO:31).

The amino acid sequence of the mature Exemplary Anti-human HSPA5 Antibody 2 light chain variable region (VL) is shown below (CDRs are underlined).

(SEQ ID NO: 18)
DIQMTQSPSSLSASVGDRVTITCRASQSIDSYLNWYQQKPGKAPKLLIYAA
SSLQSGVPSRFSGSGFGTDFTLTISRLQPEDFATYCCQQSHSLPRTFGQGT
KVEIKR

In some instances, the VL of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:18. In some instances, the VL of an anti-human HSPA5 antibody of this disclosure comprises or consists of an amino acid sequence that is identical to SEQ ID NO:18 except for 1, 2, 3, 4, 5, 6, 7, 8, 9, or 10 amino acid substitutions, deletions, and/or insertions. These amino acid substitutions, deletions, and/or insertions may be introduced to, e.g., increase the affinity, stability of the antibody or antigen-binding fragment thereof. In particular embodiments, the VL comprises one, two, or three VL CDRs (VL CDR1, VL CDR2, and VL CDR3) according to any CDR definition (e.g., Kabat, Chothia, enhanced Chothia, AbM, or contact definitions) of the VL having the amino acid sequence set forth in SEQ ID NO:18. In certain instances, the VL of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence MGVPTQVLGLLLLWLTDARC (SEQ ID NO:32).

The amino acid sequence of the mature Exemplary Anti-human HSPA5 Antibody 2 heavy chain is shown below (the three CDRs are italicized; the heavy chain constant region (IgG1) is boldened).

(SEQ ID NO: 19)
EVQLVESGGGLVHPGESLRLSCAASGFSLSGSGIHWVRQASGKGLEWLGHI
RSKSDGSATLYAASVRGRFIISRDDSTNTAYLHMSSLRNEDTAVYYCARYR
RVAVAGYTYYYYMDVWGKGSTVSVSSASTKGPSVFPLAPCSRSTSESTAAL
GCLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREEE
YNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

In some instances, the heavy chain of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:19. In some instances, the heavy chain of an anti-human HSPA5 antibody of this disclosure comprises or consists of an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the heavy chain constant region of the amino acid sequence set forth in SEQ ID NO:19. In some instances, the heavy chain of an anti-human HSPA5 antibody of this disclosure comprises or consists of an amino acid sequence that is identical to SEQ ID NO:19 except for 1, 2, 3, 4, 5, 6, 7, 8, 9, 10, 11, 12, 13, 14, 15, 16, 17, 18, 19, or 20 amino acid substitutions, deletions, and/or insertions. These amino acid substitutions, deletions, and/or insertions may be introduced to, e.g., increase the affinity, stability of the antibody or antigen-binding fragment thereof. In some instances, the heavy chain of an anti-human HSPA5 antibody of this disclosure comprises or consists of an amino acid sequence that is identical to SEQ ID NO:19 except for 1, 2, 3, 4, 5, 6, 7, 8, 9, 10, 11, 12, 13, 14, 15, 16, 17, 18, 19, or 20 amino acid substitutions, deletions, and/or insertions in the heavy chain constant region of the amino acid sequence set forth in SEQ ID NO:19. In particular embodiments of the above instances, the heavy chain comprises one, two, or three VH CDRs (VH CDR1, VH CDR2, and VH CDR3) according to any CDR definition (e.g., Kabat, Chothia, enhanced Chothia, AbM, or contact definitions) of the VH having the amino acid sequence set forth in SEQ ID NO:17. In certain embodiments of the above instances, the heavy chain of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence

(SEQ ID NO: 31)
MEWSWVFLFFLSVTTGVHS.

The amino acid sequence of the mature Exemplary Anti-human HSPA5 Antibody 2 light chain is shown below (the three CDRs are underlined; the light chain constant region (Vκ) is boldened).

(SEQ ID NO: 20)
DIQMTQSPSSLSASVGDRVTITCRASQSIDSYLNWYQQKPGKAPKLLIYAA
SSLQSGVPSRFSGSGFGTDFTLTISRLQPEDFATYCCQQSHSLPRTFGQGT
KVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSP
VTKSFNRGEC

In some instances, the light chain of an anti-human HSPA5 antibody of this disclosure comprises or consists of an amino acid sequence that is at least 75%, at least 80%, at least 85%, at least 90%, at least 95%, at least 97%, at least 98%, or at least 99% identical to the amino acid sequence set forth in SEQ ID NO:20. In some instances, the light chain of an anti-human HSPA5 antibody of this disclosure comprises or consists of an amino acid sequence that is at least 75%, at least 80%, at least 85%, at least 90%, at least 91%, at least 92%, at least 93%, at least 94%, at least 95%, at least 96%, at least 97%, at least 98%, or at least 99% identical to the heavy chain constant region of the amino acid sequence set forth in SEQ ID NO:20. In some instances, the light chain of an anti-human HSPA5 antibody of this disclosure comprises or consists of an amino acid sequence that is identical to SEQ ID NO:20 except for 1, 2, 3, 4, 5, 6, 7, 8, 9, 10, 11, 12, 13, 14, 15, 16, 17, 18, 19, or 20 amino acid substitutions, deletions, and/or insertions. These amino acid substitutions, deletions, and/or insertions may be introduced to, e.g., increase the affinity, stability of the antibody or antigen-binding fragment thereof. In some instances, the heavy chain of an anti-human HSPA5 antibody of this disclosure comprises or consists of an amino acid sequence that is identical to SEQ ID NO:20 except for 1, 2, 3, 4, 5, 6, 7, 8, 9, 10, 11, 12, 13, 14, 15, 16, 17, 18, 19, or 20 amino acid substitutions, deletions, and/or insertions in the light chain constant region of the amino acid sequence set forth in SEQ ID NO:20. In particular embodiments of the above instances, the light chain comprises one, two, or three VL CDRs (VL CDR1, VL CDR2, and VL CDR3) according to any CDR definition (e.g., Kabat, Chothia, enhanced Chothia, AbM, or contact definitions) of the VL having the amino acid sequence set forth in SEQ ID NO:18. In certain embodiments of the above instances, the light chain of an anti-human HSPA5 antibody of this disclosure further comprises a signal peptide having an amino acid sequence that is at least 75%, at least 80%, at least 85%, at least 90%, at least 95%, at least 97%, at least 98%, or at least 99% identical to the amino acid sequence

(SEQ ID NO: 32)
MGVPTQVLGLLLLWLTDARC.

In certain instances, the anti-HSPA5 antibody is not one of the commercial anti-HSPA5 antibodies (e.g., Santa Cruz, sc-376768, sc-1051, Abcam GRP78 Abl, ab12223).

Exemplary anti-HSPA5 Antibody 2 is an IgG1/kappa antibody with a heavy chain of SEQ ID NO:19 and a light chain of SEQ ID NO:20.

### Nucleic Acids Encoding Anti-HSPA5 Antibodies

The disclosure also features a nucleic acid or nucleic acids that encode any of the antibodies described above. As noted above, it is to be understood that when reference is made to anti-HSPA5 antibodies herein, that it encompasses not only whole antibodies, but also HSPA5-binding antibody fragments, minibodies, nanobodies, VHHs, and VNARs.

In specific embodiments, the nucleic acid or nucleic acids are isolated nucleic acid or nucleic acids.

In one embodiment, a nucleic acid encoding the VH of Exemplary anti-human HSPA5 Antibody 1 has the following nucleic acid sequence.

(SEQ ID NO: 21)
CAGGTGCAGCTACAGCAGTGGGGCGCAGGACTGTTGAAGCCTTCGGAGACC
CTGTCCCTCACTTGCGCTGTCTATGGTGGGTCCTTCAGTGGTTACTGGTGG
ACTTGGATCCGCCAGTCCCCAGAGAAGGGCCTGGAGTGGATTGGCGAAATC
AATCCTAGTGACACCACCAATTACAACCCGTCCCTCAAGAGTCGAGTCGCC
ATATCAAAGGACTCATCCAAGAACCAATTCTCCCTGCGGCTGTCGTCTGTG
ACCGCCGCGGACACGGCTCTCTATTTCTGTGCGAGAGGCCGTTCCACCTAC
GGGGCCAGCACTTTCTACAACTACTACTACTTTGACTCTTGGGGCCAGGGA
TCGCGGGTCGTCGTCTCTTCA

In certain embodiments, an anti-HSPA5 antibody comprises a VH encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:21. In some instances, the nucleic acid encodes an anti-HSPA5 antibody that comprises one, two, or all three VH CDRs (defined according to any CDR definition, e.g., Kabat, Chothia, enhanced Chothia, AbM, contact) of Exemplary anti-human HSPA5 Antibody 1.

In one embodiment, a nucleic acid encoding the heavy chain of Exemplary anti-human HSPA5 Antibody 1 has the following nucleic acid sequence.

(SEQ ID NO: 25)
CAGGTGCAGCTACAGCAGTGGGGCGCAGGACTGTTGAAGCCTTCGGAGACC
CTGTCCCTCACTTGCGCTGTCTATGGTGGGTCCTTCAGTGGTTACTGGTGG
ACTTGGATCCGCCAGTCCCCAGAGAAGGGCCTGGAGTGGATTGGCGAAATC
AATCCTAGTGACACCACCAATTACAACCCGTCCCTCAAGAGTCGAGTCGCC
ATATCAAAGGACTCATCCAAGAACCAATTCTCCCTGCGGCTGTCGTCTGTG
ACCGCCGCGGACACGGCTCTCTATTTCTGTGCGAGAGGCCGTTCCACCTAC
GGGGCCAGCACTTTCTACAACTACTACTACTTTGACTCTTGGGGCCAGGGA
TCGCGGGTCGTCGTCTCTTCAGCCTCCACCAAGGGCCCATCGGTCTTCCCC
CTGGCACCCTCCTCCAAGAGCACCTCTGGGGGCACAGCGGCCCTGGGCTGC
CTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACC
CAGACCTACATCTGCAACGTGAATCACAAGCCCAGCAACACCAAGGTGGAC
AAGAAAGTTGAGCCCAAATCTTGTGACAAAACTCACACATGCCCACCGTGC
CCAGCACCTGAACTCCTGGGGGGACCGTCAGTCTTCCTCTTCCCCCCAAAA
CCCAAGGACACCCTCATGATCTCCCGGACCCCTGAGGTCACATGCGTGGTG
GTGGACGTGAGCCACGAAGACCCTGAGGTCAAGTTCAACTGGTACGTGGAC
GGCGTGGAGGTGCATAATGCCAAGACAAAGCCGCGGGAGGAGGAGTACAAC
AGCACGTACCGTGTGGTCAGCGTCCTCACCGTCCTGCACCAGGACTGGCTG
AATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGTG
TACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAG
AGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGG
TGGCAGCAGGGGAACGTCTTCTCATGCTCCGTGATGCATGAGGCTCTGCAC
AACCACTACACGCAGAAGAGCCTCTCCCTGTCTCCGGGTAAA.

In certain embodiments, an anti-HSPA5 antibody comprises a heavy chain encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:25. In some instances, the nucleic acid encodes an anti-HSPA5 antibody that comprises one, two, or all three VH CDRs (defined according to any CDR definition, e.g., Kabat, Chothia, enhanced Chothia, AbM, contact) of Exemplary anti-human HSPA5 Antibody 1.

In certain embodiments, the VH or heavy chain further comprises a signal peptide sequence that is encoded by a nucleic acid that is at least 80%, at least 85%, at least 90%, at least 91%, at least 92%, at least 93%, at least 94%, at least 95%, at least 96%, at least 97%, at least 98%, or at least 99% identical to the nucleic acid sequence:

(SEQ ID NO: 41)
ATGGAATGGAGCTGGGTCTTTCTCTTCTTCCTGTCAGTAACTACAGGTGTC
CACTCC

In one embodiment, a nucleic acid encoding the VL of Exemplary anti-human HSPA5 Antibody 1 has the following nucleic acid sequence.

(SEQ ID NO: 22)
GACATCCAGATGACCCAGTCTCCATCCACCCTGTCTGCTTCTGTCGGAGAC
ACAATTACCATCACTTGCCGGGCCAGTCAGAAGATTGATAGGTGGTTGGCC
TGGTATCAGCAGAAACCAGGCAAAGCCCCTAAACTTCTCATATATAAGGCC
TCTAATTTAGAGAGTGGGGTCCCACCTAGGCTCAGCGGCACTGGATCTGGG
ACAGACTTCACTCTCACTATCAAGAGCCTGCAGCCGGATGATTTTGCAACG
TATTTCTGTCAACAGTATCAGAATTACCCCTACACTTTTGGCCCGGGGACC
AAGCTGGAGATGAGACGA

In certain embodiments, an anti-HSPA5 antibody comprises a VL encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:22. In some instances, the nucleic acid encodes an anti-HSPA5 antibody that comprises one, two, or all three VL CDRs (defined according to any CDR definition, e.g., Kabat, Chothia, enhanced Chothia, AbM, contact) of Exemplary anti-human HSPA5 Antibody 1.

In one embodiment, a nucleic acid encoding the light chain of Exemplary anti-human HSPA5 Antibody 1 has the following nucleic acid sequence.

(SEQ ID NO: 26)
GACATCCAGATGACCCAGTCTCCATCCACCCTGTCTGCTTCTGTCGGAGAC
ACAATTACCATCACTTGCCGGGCCAGTCAGAAGATTGATAGGTGGTTGGCC
TGGTATCAGCAGAAACCAGGCAAAGCCCCTAAACTTCTCATATATAAGGCC
TCTAATTTAGAGAGTGGGGTCCCACCTAGGCTCAGCGGCACTGGATCTGGG
ACAGACTTCACTCTCACTATCAAGAGCCTGCAGCCGGATGATTTTGCAACG
TATTTCTGTCAACAGTATCAGAATTACCCCTACACTTTTGGCCCGGGGACC
AAGCTGGAGATGAGACGAACTGTGGCTGCACCATCTGTCTTCATCTTCCCG
CCATCTGATGAGCAGTTGAAATCTGGAACTGCCTCTGTCGTGTGCCTGCTG
AATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAG
AAACACAAAGTCTACGCCTGCGAAGTCACCCATCAGGGCCTGAGTTCGCCC
GTCACAAAGAGCTTCAACAGGGGAGAGTGT

In certain embodiments, an anti-HSPA5 antibody comprises a light chain encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:26. In some instances, the nucleic acid encodes an anti-HSPA5 antibody that comprises one, two, or all three VL CDRs (defined according to any CDR definition, e.g., Kabat, Chothia, enhanced Chothia, AbM, contact) of Exemplary anti-human HSPA5 Antibody 1.

In certain embodiments, the VL or light chain further comprises a signal peptide sequence that is encoded by a nucleic acid that is at least 80%, at least 85%, at least 90%, at least 91%, at least 92%, at least 93%, at least 94%, at least 95%, at least 96%, at least 97%, at least 98%, or at least 99% identical to the nucleic acid sequence:

(SEQ ID NO: 42)
ATGGGTGTGCCCACTCAGGTCCTGGGGTTGCTGCTGCTGTGGCTTACAGAT
GCCAGATGT

In one embodiment, a nucleic acid encoding the VH of Exemplary anti-human HSPA5 Antibody 2 has the following nucleic acid sequence.

(SEQ ID NO: 23)
GAGGTGCAGCTGGTGGAGTCTGGGGGAGGCTTGGTCCACCCTGGGGAGTCC
CTGAGACTCTCCTGTGCAGCCTCTGGGTTTAGTTTGAGTGGCTCTGGTATA
CACTGGGTCCGCCAGGCTTCCGGAAAAGGGCTGGAGTGGCTTGGCCATATT
AGAAGCAAAAGTGACGGTTCCGCGACATTGTATGCTGCGTCGGTGAGAGGC
AGGTTCATTATCTCCAGAGATGATTCAACGAATACGGCGTATCTGCACATG
AGCAGCCTGAGAAACGAGGACACGGCCGTCTATTACTGCGCTAGATACAGA
CGAGTGGCAGTGGCAGGTTACACCTACTACTACTACATGGACGTCTGGGGC
AAAGGGTCCACGGTCTCCGTCTCCTCA

In certain embodiments, an anti-HSPA5 antibody comprises a VH encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:23. In some instances, the nucleic acid encodes an anti-HSPA5 antibody that comprises one, two, or all three VH CDRs (defined according to any CDR definition, e.g., Kabat, Chothia, enhanced Chothia, AbM, contact) of Exemplary anti-human HSPA5 Antibody 2.

In one embodiment, a nucleic acid encoding the heavy chain of Exemplary anti-human HSPA5 Antibody 2 has the following nucleic acid sequence.

(SEQ ID NO: 27)
GAGGTGCAGCTGGTGGAGTCTGGGGGAGGCTTGGTCCACCCTGGGGAGTCC
CTGAGACTCTCCTGTGCAGCCTCTGGGTTTAGTTTGAGTGGCTCTGGTATA
CACTGGGTCCGCCAGGCTTCCGGAAAAGGGCTGGAGTGGCTTGGCCATATT
AGAAGCAAAAGTGACGGTTCCGCGACATTGTATGCTGCGTCGGTGAGAGGC
AGGTTCATTATCTCCAGAGATGATTCAACGAATACGGCGTATCTGCACATG
AGCAGCCTGAGAAACGAGGACACGGCCGTCTATTACTGCGCTAGATACAGA
CGAGTGGCAGTGGCAGGTTACACCTACTACTACTACATGGACGTCTGGGGC
AAAGGGTCCACGGTCTCCGTCTCCTCAGCCTCCACCAAGGGCCCATCGGTC
TTCCCCCTGGCGCCCTGCTCCAGGAGCACCTCCGAGAGCACAGCGGCCCTG
GGCTGCCTGGTCAAGGACTACTTCCCCGAACCGGTGACGGTGTCGTGGAAC
TCAGGCGCCCTGACCAGCGGCGTGCACACCTTCCCGGCTGTCCTACAGTCC
TCAGGACTCTACTCCCTCAGCAGCGTGGTGACCGTGCCCTCCAGCAGCTTG
GGCACCCAGACCTACATCTGCAACGTGAATCACAAGCCCAGCAACACCAAG
GTGGACAAGAAAGTTGAGCCCAAATCTTGTGACAAAACTCACACATGCCCA
CCGTGCCCAGCACCTGAACTCCTGGGGGGACCGTCAGTCTTCCTCTTCCCC
CCAAAACCCAAGGACACCCTCATGATCTCCCGGACCCCTGAGGTCACATGC
GTGGTGGTGGACGTGAGCCACGAAGACCCTGAGGTCAAGTTCAACTGGTAC
GTGGACGGCGTGGAGGTGCATAATGCCAAGACAAAGCCGCGGGAGGAGGAG
TACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCA
CAGGTGTACACCCTGCCCCCATCCCGGGATGAGCTGACCAAGAACCAGGTC
AGCCTGACCTGCCTGGTCAAAGGCTTCTATCCCAGCGACATCGCCGTGGAG
TGGGAGAGCAATGGGCAGCCGGAGAACAACTACAAGACCACGCCTCCCGTG
CTGGACTCCGACGGCTCCTTCTTCCTCTACAGCAAGCTCACCGTGGACAAG
AGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA.

In certain embodiments, an anti-HSPA5 antibody comprises a heavy chain encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:27. In some instances, the nucleic acid encodes an anti-HSPA5 antibody that comprises one, two, or all three VH CDRs (defined according to any CDR definition, e.g., Kabat, Chothia, enhanced Chothia, AbM, contact) of Exemplary anti-human HSPA5 Antibody 2.

In certain embodiments, the VH or heavy chain further comprises a signal peptide sequence that is encoded by a nucleic acid that is at least 80%, at least 85%, at least 90%, at least 91%, at least 92%, at least 93%, at least 94%, at least 95%, at least 96%, at least 97%, at least 98%, or at least 99% identical to the nucleic acid sequence:

(SEQ ID NO: 41)
ATGGAATGGAGCTGGGTCTTTCTCTTCTTCCTGTCAGTAACTACAGGTGTC
CACTCC

In one embodiment, a nucleic acid encoding the VL of Exemplary anti-human HSPA5 Antibody 2 has the following nucleic acid sequence.

(SEQ ID NO: 24)
GACATCCAGATGACCCAGTCTCCATCCTCCCTGTCTGCATCTGTAGGAGAC
AGAGTCACCATCACTTGCCGGGCAAGTCAGAGCATTGACAGCTATTTAAAT
TGGTATCAGCAGAAACCAGGGAAAGCCCCTAAGCTCCTGATCTATGCTGCA
TCCAGTTTGCAAAGTGGGGTCCCATCAAGGTTCAGTGGCAGTGGATTTGGG
ACAGATTTCACTCTCACCATCAGCAGGCTGCAACCTGAGGATTTTGCAACT
TACTGCTGTCAACAGAGTCACAGTCTCCCCCGGACGTTCGGCCAAGGGACC
AAGGTGGAAATCAAACGA

In certain embodiments, an anti-HSPA5 antibody comprises a VL encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:24. In some instances, the nucleic acid encodes an anti-HSPA5 antibody that comprises one, two, or all three VL CDRs (defined according to any CDR definition, e.g., Kabat, Chothia, enhanced Chothia, AbM, contact) of Exemplary anti-human HSPA5 Antibody 2.

In one embodiment, a nucleic acid encoding the light chain of Exemplary anti-human HSPA5 Antibody 2 has the following nucleic acid sequence.

(SEQ ID NO: 28)
GACATCCAGATGACCCAGTCTCCATCCTCCCTGTCTGCATCTGTAGGAGAC
AGAGTCACCATCACTTGCCGGGCAAGTCAGAGCATTGACAGCTATTTAAAT
TGGTATCAGCAGAAACCAGGGAAAGCCCCTAAGCTCCTGATCTATGCTGCA
TCCAGTTTGCAAAGTGGGGTCCCATCAAGGTTCAGTGGCAGTGGATTTGGG
ACAGATTTCACTCTCACCATCAGCAGGCTGCAACCTGAGGATTTTGCAACT
TACTGCTGTCAACAGAGTCACAGTCTCCCCCGGACGTTCGGCCAAGGGACC
AAGGTGGAAATCAAACGAACTGTGGCTGCACCATCTGTCTTCATCTTCCCG
CCATCTGATGAGCAGTTGAAATCTGGAACTGCCTCTGTTGTGTGCCTGCTG
AATAACTTCTATCCCAGAGAGGCCAAAGTACAGTGGAAGGTGGATAACGCC
CTCCAATCGGGTAACTCCCAGGAGAGTGTCACAGAGCAGGACAGCAAGGAC
AGCACCTACAGCCTCAGCAGCACCCTGACGCTGAGCAAAGCAGACTACGAG
AAACACAAAGTCTACGCCTGCGAAGTCACCCATCAGGGCCTGAGTTCGCCC
GTCACAAAGAGCTTCAACAGGGGAGAGTGT

In certain embodiments, an anti-HSPA5 antibody comprises a light chain encoded by a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence set forth in SEQ ID NO:28. In some instances, the nucleic acid encodes an anti-HSPA5 antibody that comprises one, two, or all three VL CDRs (defined according to any CDR definition, e.g., Kabat, Chothia, enhanced Chothia, AbM, contact) of Exemplary anti-human HSPA5 Antibody 2.

In certain embodiments, the VL or light chain further comprises a signal peptide sequence that is encoded by a nucleic acid that is at least 80%, at least 85%, at least 90%, at least 91%, at least 92%, at least 93%, at least 94%, at least 95%, at least 96%, at least 97%, at least 98%, or at least 99% identical to the nucleic acid sequence:

(SEQ ID NO: 42)
ATGGGTGTGCCCACTCAGGTCCTGGGGTTGCTGCTGCTGTGGCTTACAGAT
GCCAGATGT

The nucleic acid or nucleic acids can be, e.g., DNA or cDNA. In the case of DNA or cDNA, a polynucleotide comprising a nucleic acid which encodes a polypeptide can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit B-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention can be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

### Methods of Obtaining Anti-HSPA5 Antibodies

Numerous methods are available for obtaining antibodies, particularly human antibodies. One exemplary method includes screening protein expression libraries, e.g., phage or ribosome display libraries. Phage display is described, for example, in U.S. Pat. No. 5,223,409; Smith, Science 228:1315-1317 (1985); WO 92/18619; WO 91/17271; WO 92/20791; WO 92/15679; WO 93/01288; WO 92/01047; WO 92/09690; and WO 90/02809. The display of Fab's on phage is described, e.g., in U.S. Pat. Nos. 5,658,727; 5,667,988; and 5,885,793.

In addition to the use of display libraries, other methods can be used to obtain a HSPA5-binding antibody. For example, the human HSPA5 protein or a peptide thereof can be used as an antigen in a non-human animal, e.g., a rodent, e.g., a mouse, hamster, or rat. In addition, cells transfected with a cDNA encoding human HSPA5 can be injected into a non-human animal as a means of producing antibodies that effectively bind the cell surface human HSPA5 protein.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al., Nature Genetics 7:13-21 (1994), U.S. 2003-0070185, WO 96/34096, and WO 96/33735. Such methods allow the preparation of fully human anti-HSPA5 antibodies.

In another embodiment, a monoclonal antibody is obtained from the non-human animal, and then modified, e.g., humanized or deimmunized. Winter describes an exemplary CDR-grafting method that may be used to prepare humanized antibodies described herein (U.S. Pat. No. 5,225,539). All or some of the CDRs of a particular human antibody may be replaced with at least a portion of a non-human antibody. It may only be necessary to replace the CDRs required for binding or binding determinants of such CDRs to arrive at a useful humanized antibody that binds to human HSPA5.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16: 237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

A non-human HSPA5-binding antibody may also be modified by specific deletion of human T cell epitopes or “deimmunization” by the methods disclosed in WO 98/52976 and WO 00/34317. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible, conservative substitutions are made. Often, but not exclusively, an amino acid common to a position in human germline antibody sequences may be used. After the deimmunizing changes are identified, nucleic acids encoding V_{H} and V_{L} can be constructed by mutagenesis or other synthetic methods (e.g., de novo synthesis, cassette replacement, and so forth). A mutagenized variable sequence can, optionally, be fused to a human constant region, e.g., human IgG1 or kappa constant regions.

In some cases, a potential T cell epitope will include residues known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs can be eliminated by substitutions outside the CDRs. In some cases, an alteration within a CDR is the only option, and thus variants with and without this substitution can be tested. In other cases, the substitution required to remove a potential T cell epitope is at a residue position within the framework that might be critical for antibody binding. In these cases, variants with and without this substitution are tested. Thus, in some cases several variant deimmunized heavy and light chain variable regions are designed and various heavy/light chain combinations are tested to identify the optimal deimmunized antibody. The choice of the final deimmunized antibody can then be made by considering the binding affinity of the different variants in conjunction with the extent of deimmunization, particularly, the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271. Still another method is termed “humaneering” and is described, for example, in U.S. 2005/0008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237 (based on EU numbering). Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005/0037000.

### Affinity Maturation

In one embodiment, an anti-HSPA5 antibody or antigen-binding fragment thereof is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used. Methods of effecting affinity maturation include random mutagenesis (e.g., Fukuda et al., Nucleic Acids Res., 34:e127 (2006); targeted mutagenesis (e.g., Rajpal et al., Proc. Natl. Acad. Sci. USA, 102:8466-71 (2005); shuffling approaches (e.g., Jermutus et al., Proc. Natl. Acad. Sci. USA, 98:75-80 (2001); and in silico approaches (e.g., Lippow et al., Nat. Biotechnol., 25:1171-6 (2005).

In some implementations, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criterion for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half-life by providing a salvage receptor binding epitope.

In one embodiment, an anti-HSPA5 antibody has one or more CDR sequences (e.g., a Chothia, an enhanced Chothia, or Kabat CDR) that differ from those of the Exemplary Anti-HSPA5 Antibody 1. In one embodiment, an anti-HSPA5 antibody has one or more CDR sequences that differ from those of the Exemplary Anti-HSPA5 Antibody 2. CDR sequences that differ from those of the Exemplary Anti-HSPA5 Antibody 1 or 2 include amino acid changes, such as substitutions of 1, 2, 3, or 4 amino acids if a CDR is 5-7 amino acids in length, or substitutions of 1, 2, 3, 4, or 5, of amino acids in the sequence of a CDR if a CDR is 8 amino acids or greater in length. The amino acid that is substituted can have similar charge, hydrophobicity, or stereochemical characteristics. In some embodiments, the amino acid substitution(s) is a conservative substitution. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In other embodiments, the amino acid substitution(s) is a non-conservative substitution. The antibody or antibody fragments thereof that contain the substituted CDRs can be screened to identify antibodies of interest.

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J. Immun., 147:2657-62 (1991); Morgan et al., Immunology, 86:319-24 (1995)), or changing the species from which the constant region is derived.

Antibodies with Reduced Effector Function

The interaction of antibodies and antibody-antigen complexes with cells of the immune system triggers a variety of responses, referred to herein as effector functions. Immune-mediated effector functions include two major mechanisms: antibody-dependent cell-mediated cytotoxicity (ADCC) and complement-dependent cytotoxicity (CDC). Both of them are mediated by the constant region of the immunoglobulin protein. The antibody Fc domain is, therefore, the portion that defines interactions with immune effector mechanisms.

IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. These responses can provoke unwanted side effects such as inflammation and the elimination of antigen-bearing cells. Accordingly, the present invention further relates to HSPA5-binding proteins, including antibodies, with reduced effector functions.

Effector function of an anti-HSPA5 antibody of the present invention may be determined using one of many known assays. The anti-HSPA5 antibody's effector function may be reduced relative to a second anti-HSPA5 antibody. In some embodiments, the second anti-HSPA5 antibody may be any antibody that binds HSPA5 specifically. In other embodiments, the second HSPA5-specific antibody may be Exemplary Anti-HSPA5 Antibody 1 or Exemplary Anti-HSPA5 Antibody 2. In other embodiments, where the anti-HSPA5 antibody of interest has been modified to reduce effector function, the second anti-HSPA5 antibody may be the unmodified or parental version of the antibody.

Effector functions include ADCC, whereby antibodies bind Fc receptors on cytotoxic T cells, natural killer (NK) cells, or macrophages leading to cell death, and CDC, which is cell death induced via activation of the complement cascade (reviewed in Daeron, Annu. Rev. Immunol., 15:203-234 (1997); Ward and Ghetie, Therapeutic Immunol., 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195).

Effector functions can be avoided by using antibody fragments lacking the Fc domain such as Fab, Fab′2, or single chain Fv. An alternative is to use the IgG4 subtype antibody, which binds to FcγRI but which binds poorly to C1q and FcγRII and RIII. However, IgG4 antibodies may form aggregates since they have poor stability at low pH compared with IgG1 antibodies. The stability of an IgG4 antibody can be improved by substituting arginine at position 409 (according to the EU index proposed by Kabat et al., Sequences of proteins of immunological interest, 1991, 5^{th}) with any one of: lysine, methionine, threonine, leucine, valine, glutamic acid, asparagine, phenylalanine, tryptophan, or tyrosine. Alternatively, and or in addition, the stability of an IgG4 antibody can be improved by substituting a CH3 domain of an IgG4 antibody with a CH3 domain of an IgG1 antibody, or by substituting the CH2 and CH3 domains of IgG4 with the CH2 and CH3 domains of IgG1. Accordingly, the anti-HSPA5 antibodies of the present invention that are of IgG4 isotype can include modifications at position 409 and/or replacement of the CH2 and/or CH3 domains with the IgG1 domains so as to increase stability of the antibody while decreasing effector function. The IgG2 subtype also has reduced binding to Fc receptors, but retains significant binding to the H131 allotype of FcγRIIa and to C1q. Thus, additional changes in the Fc sequence may be required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FcεR, for IgA as FcαR and so on. Three subclasses of FcγR have been identified: FcγRI (CD64), FcγRII (CD32) and FcγRIII (CD16). Both FcγRII and FcγRIII have two types: FcγRIIa (CD32a) and FcγRIIB (CD32b); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms IIa, IIb1, IIb2 IIb3, and IIc.

The binding site on human and murine antibodies for FcγR has been previously mapped to the so-called “lower hinge region” consisting of residues G233-S239 (EU index numbering as in Kabat et al., Sequences of Proteins of Immunological Interest, 5th Ed. Public Health Service, National Institutes of Health, Bethesda, Md. (1991), Woof et al., Molec. Immunol. 23:319-330 (1986); Duncan et al., Nature 332:563 (1988); Canfield and Morrison, J. Exp. Med. 173:1483-1491 (1991); Chappel et al., Proc. Natl. Acad. Sci USA 88:9036-9040 (1991)). Of residues G233-5239, P238 and S239 are among those cited as possibly being involved in binding. Other residues involved in binding to FcγR are: G316-K338 (Woof et al., Mol. Immunol., 23:319-330 (1986)); K274-R301 (Sarmay et al., Molec. Immunol. 21:43-51 (1984)); Y407-R416 (Gergely et al., Biochem. Soc. Trans. 12:739-743 (1984) and Shields et al., J Biol Chem 276: 6591-6604 (2001), Lazar G A et al., Proc Natl Acad Sci 103: 4005-4010 (2006)); N297; T299; E318; L234-5239; N265-E269; N297-T299; and A327-I332. These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-HSPA5 antibodies of the present invention include modifications of one or more of the aforementioned residues to decrease effector function as needed.

Another approach for altering monoclonal antibody effector function include mutating amino acids on the surface of the monoclonal antibody that are involved in effector binding interactions (Lund, J., et al. (1991) J. Immunol. 147(8): 2657-62; Shields, R. L. et al. (2001) J. Biol. Chem. 276(9): 6591-604).

To reduce effector function, one can use combinations of different subtype sequence segments (e.g., IgG2 and IgG4 combinations) to give a greater reduction in binding to Fcγ receptors than either subtype alone (Armour et al., Eur. J. Immunol., 29:2613-1624 (1999); Mol. Immunol., 40:585-593 (2003)). A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260.

Anti-HSPA5 antibodies of the present invention with reduced effector function include antibodies with reduced binding affinity for one or more Fc receptors (FcRs) relative to a parent or non-variant anti-HSPA5 antibody. Accordingly, anti-HSPA5 antibodies with reduced FcR binding affinity includes anti-HSPA5 antibodies that exhibit a 1.5-fold, 2-fold, 2.5-fold, 3-fold, 4-fold, 5-fold, 10-fold, 20-fold, or 25-fold or higher decrease in binding affinity to one or more Fc receptors compared to a parent or non-variant anti-HSPA5 antibody. In some embodiments, an anti-HSPA5 antibody with reduced effector function binds to an FcR with about 10-fold less affinity relative to a parent or non-variant antibody. In other embodiments, an anti-HSPA5 antibody with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-HSPA5 antibody with reduced effector function exhibits a 1.5-fold, 2-fold, 2.5-fold, 3-fold, 4-fold, or 5-fold or higher decrease in binding affinity to FcγRIIa.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus, the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J. Immunol. Methods, 202:163 (1996), may be performed.

It has been proposed that various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. Nos. 5,648,260, and 5,624,821).

Anti-HSPA5 antibodies with reduced C1q binding can comprise an amino acid substitution at one, two, three, or four of amino acid positions 270, 322, 329 and 331 of the human IgG Fc region, where the numbering of the residues in the IgG Fc region is that of the EU index as in Kabat. As an example in IgG1, two mutations in the COOH terminal region of the CH2 domain of human IgG1—K322A and P329A—do not activate the CDC pathway and were shown to result in more than a 100-fold decrease in C1q binding (U.S. Pat. No. 6,242,195).

Accordingly, in certain embodiments, an anti-HSPA5 antibody of the present invention exhibits reduced binding to a complement protein relative to a second anti-HSPA5 antibody. In certain embodiments, an anti-HSPA5 antibody of the invention exhibits reduced binding to C1q by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to a second anti-HSPA5 antibody.

Thus, in certain embodiments of the invention, one or more of these residues may be modified, substituted, or removed or one or more amino acid residues may be inserted so as to decrease CDC activity of the anti-HSPA5 antibodies provided herein.

In certain other embodiments, the present invention provides an anti-HSPA5 antibody that exhibits reduced binding to one or more FcR receptors but that maintains its ability to bind complement (e.g., to a similar or, in some embodiments, to a lesser extent than a native, non-variant, or parent anti-HSPA5 antibody). Accordingly, an anti-HSPA5 antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind C1q and activate the complement cascade to at least some degree will reduce the risk of thromboembolic events while maintaining perhaps desirable effector functions. In alternative embodiments, an anti-HSPA5 antibody of the present invention exhibits reduced binding to one or more FcRs but maintains its ability to bind one or more other FcRs. See, for example, US 2007/0009523, 2006/0194290, 2005/0233382, 2004/0228856, and 2004/0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an anti-HSPA5 antibody may be modulated by altering properties of the constant region, and the Fc region in particular. In certain embodiments, the anti-HSPA5 antibody having decreased effector function is compared with a second antibody with effector function and which may be a non-variant, native, or parent antibody comprising a native constant or Fc region that mediates effector function.

A native constant region comprises an amino acid sequence identical to the amino acid sequence of a constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. The variant constant region can have decreased effector function.

Antibodies with decreased effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns, may be achieved by manipulating the host cell and cell culture and expression conditions by which the antibody is produced.

In some embodiments, the disclosure provides an anti-HSPA5 antibody comprising a VL sequence comprising SEQ ID NO:8 and a VH sequence comprising SEQ ID NO:7, the antibody further comprising an Fc region (e.g., IgG4 Fc region) or a variant Fc region that confers reduced effector function compared to a native or parental Fc region. In other embodiments, the disclosure provides an anti-HSPA5 antibody comprising a VL sequence comprising SEQ ID NO:18 and a VH sequence comprising SEQ ID NO:17, the antibody further comprising an Fc region (e.g., IgG4 Fc region) or a variant Fc region that confers reduced effector function compared to a native or parental Fc region.

Methods of generating any of the aforementioned anti-HSPA5 antibody variants comprising amino acid substitutions are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

### Methods of Producing Antibodies

Antibodies or antigen binding fragments thereof may be produced in bacterial or eukaryotic cells. Some antibodies, e.g., Fab's, can be produced in bacterial cells, e.g., E. coli cells. Antibodies or antigen binding fragments thereof can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hanseula, or Saccharomyces. In one embodiment, the anti-HSPA5 antibodies described herein are produced in the dihydrofolate reductase-deficient Chinese hamster ovary (CHO) cell line, DG44. In another embodiment, the anti-HSPA5 antibodies described herein are produced in the DG44i cell line. To produce the antibody or antigen binding fragments thereof of interest, a polynucleotide or polynucleotides encoding the antibody are constructed, introduced into an expression vector or expression vectors, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector or vectors should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, recombinant expression vectors encoding the antibody heavy chain and the antibody light chain of an anti-HSPA5 antibody, respectively (e.g., Exemplary Anti-HSPA5 Antibody 1, Exemplary anti-HSPA5 Antibody 2) are introduced into dhfr⁻ CHO cells by calcium phosphate-mediated transfection. In a specific embodiment, the dhfr− CHO cells are cells of the DG44 cell line, such as DG44i (see, e.g., Derouaz et al., Biochem Biophys Res Commun., 340(4):1069-77 (2006)). Within the recombinant expression vectors, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vectors also carry a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Characterization of the Antibodies

The HSPA5-binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-HSPA5 antibody) and a target (e.g., HSPA5) can be analyzed using the OCTET® systems. In this method, one of several variations of instruments (e.g., OCTET® QKe and QK), made by the FortéBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-HSPA5 antibody) and a target (e.g., HSPA5) can be analyzed using Surface Plasmon Resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to human HSPA5 using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

The Examples of this disclosure provide additional methods of evaluating the anti-HSPA5 antibodies described herein (e.g., assessing effect of the antibodies on BBB integrity by using solute permeability with 10 kDa dextran; assessing structural change of tight junctions in BMECs by using area fraction of claudin-5 expression; assessing ICAM-1 expression on BMECs in the presence of the anti-HSPA5 antibodies; determining the ability of the anti-HSPA5 antibodies to mediate nuclear translocation of NFκB p65).

Additional general guidance for evaluating antibodies, e.g., Western blots and immunoprecipitation assays, can be found in Antibodies: A Laboratory Manual, ed. by Harlow and Lane, Cold Spring Harbor press (1988)).

The function and/or activities of the anti-HSPA5 antibodies described herein (e.g., Exemplary anti-HSPA5 Antibody 1, Exemplary anti-HSPA5 Antibody 2) can be compared with other reference or comparator anti-HSPA5 antibodies.

### Methods of Delivering an Agent to a Component of CNS

The distribution of macromolecules throughout the body is generally diffusion mediated with macromolecules in the blood diffusing into the tissues across highly fenestrated endothelial cell linings of the capillary vasculature. Free diffusion of macromolecules does not exist in the highly vascularized brain. Brain capillary endothelial cells lack the fenestrations seen in the rest of the circulatory system and have highly specialized endothelial cell tight intracellular junctions. These tight junctions serve to prevent the free diffusion of molecules of greater than 400 kDa from the luminal to the abluminal side of the capillary. In addition, the capillaries contain various transporter systems such as the Organic Anion Transporters (OATS) and Multi-Drug Resistance (MDR) systems that actively establish transportation gradients of molecules that might otherwise diffuse through endothelial cells. The combination of the restrictive barriers prevents the entrance of adventitious agents, including toxins and viruses for example, as well as restricting the diffusion of therapeutic entities. In addition, these restrictive blood brain barriers (BBB) effectively block the passive delivery of potentially therapeutic proteins, peptides and small molecules into the brain parenchyma at pharmacologically therapeutic doses.

This disclosure provides a method to achieve transport of an agent (e.g., a therapeutic molecule) across the BBB endothelial cells. This method relies on the ability of anti-HSPA5 antibodies to increase BBB permeability. In certain instances, the anti-HSPA5 antibodies alter tight junctions to increase BBB permeability. Thus, one can introduce, or enhance the delivery of, an agent of interest into the brain, spinal cord, or other component of the central nervous system by administering the agent of interest with the anti-HSPA5 antibody. The agent and the anti-HSPA5 antibody can be administered to a human subject sequentially or simultaneously. In certain instances, the agent of interest is administered before administering the anti-HSPA5 antibody. In other embodiments, the anti-HSPA5 antibody is administered to the subject prior to administration of the agent of interest. In some instances, the agent and the anti-HSPA5 antibody are administered about 10 minutes, 20 minutes 30 minutes, 45 minutes, 1 hour, 2 hours, 3 hours, 4 hours, 5 hours, 6 hours, 7 hours, 8 hours, 9 hours, 10 hours, 11, hours, 12 hours, 24 hours, 30 hours, 36 hours, 42 hours, 48 hours, 2.5 days, 3 days, 3.5 days, 4 days, 4.5 days, 5 days, 5.5 days, 6 days, or 1 week apart from one another.

In some instances, the agent(s) of interest is/are linked or connected to the anti-HSPA5 antibody. In one embodiment of the invention, an agent (e.g., a pharmaceutically active moiety) is operably linked via a linker peptide to the C-terminus of an Fc domain of the anti-HSPA5 antibody. In other embodiments, two or more agents (e.g., pharmaceutically active moieties) are linked to each other (e.g., via a linker peptide) in series. In one embodiment, the tandem array of pharmaceutically active moieties is operably linked via a linker peptide to the C-terminus of an Fc domain of the anti-HSPA5 antibody. Other methods of conjugating, linking and coupling an antibody to a pharmacologically active compound(s) are well known in the field. For example, see, Wu A M, Nat. Biotechnol., 23(9):1137-46 (2005); and Trail P A, Cancer Immunol Immunother., 52(5):328-37 (2003); Saito G, Adv Drug Deliv Rev., 55(2):199-215 (2003). Some agents may lack suitable functional groups to which the anti-HSPA5 antibody can be linked. In one embodiment, an agent, e.g., a drug or prodrug is attached to the anti-HSPA5 antibody through a linking molecule. The linking molecule may be cleavable or non-cleavable. In one embodiment, the cleavable linking molecule is a redox-cleavable linking molecule, such that the linking molecule is cleavable in environments with a lower redox potential, such as the cytoplasm and other regions with higher concentrations of molecules with free sulfhydryl groups. Examples of linking molecules that may be cleaved due to a change in redox potential include those containing disulfides.

Depending on the disease or disorder or condition targeted, a variety of agents, e.g., pharmacologically active agents, can be delivered successfully in vivo using anti-HSPA5 antibodies. As used herein, the term “pharmaceutically active agent” refers to any moiety or compound useful in treating or ameliorating the effects of a disease or disorder. For example, diseases or disorders including neurodegenerative diseases such as, Alzheimer's disease, Parkinson's disease, Frontotemporal lobar degeneration (FTLD), a Lewy body dementia (e.g., dementia with Lewy bodies (DLB) and Parkinson's disease with dementia), Huntington's disease, amyotrohpic lateral sclerosis (ALS, Lou Gehrig's disease), motor neuron disease (MND), spinocerebellar ataxia (SCA), spinal muscular atrophy (SMA), progressive supranuclear palsy (PSP), multiple sclerosis, vascular dementia, neuropathic pain, traumatic brain injury, Guillain-Barré syndrome, and chronic inflammatory demyelinating polyneuropathy (CIDP), can be targeted.

In certain instances, an agent of interest for transporting into the brain, spinal cord, or a component of the CNS includes a drug, a prodrug, an antibody, an antigen-binding fragment of an antibody or portion thereof (e.g., F(ab), a VH domain, a VL domain, a humanized VNAR, a humanized VHH, scFv, sc(Fv)2, diabody), a chemotherapeutic agent, an siRNA, a degron, a ligand binding portion of a receptor, a receptor binding portion of a ligand, an enzyme, a peptide, a protein, a protein mimotope, a stapled/stitched polypeptide, a nucleic acid (e.g., a polynucleotide, an oligonucleotide), a neurotrophic factor, a growth factor, a neurotransmitter, a neuromodulator, an antibiotic, an antiviral agent, an antifungal agent, an imaging or detectable agent, and a radioisotope.

Exemplary classes of drugs that can be transported, or whose transport can be enhanced, by the methods of this disclosure include: drugs acting at synaptic and neuroeffector junctional sites; general and local analgesics and anesthetics such as opioid analgesics and antagonists; hypnotics and sedatives; drugs for the treatment of psychiatric disorders such as depression, schizophrenia; anti-epileptics and anticonvulsants; drugs for the treatment of Huntington's disease, Alzheimer's disease, Parkinson's disease; neuroprotective agents (such as excitatory amino acid antagonists and neurotropic factors) and neuroregenerative agents; trophic factors such as brain derived neurotrophic factor, ciliary neurotrophic factor, or nerve growth factor; drugs aimed at the treatment of CNS trauma or stroke; drugs for the treatment of addiction and drug abuse; autacoids and anti-inflammatory drugs; chemotherapeutic agents; drugs for parasitic infections and microbial diseases; immunosuppressive agents and anti-cancer drugs; hormones and hormone antagonists; heavy metals and heavy metal antagonists; antagonists for non-metallic toxic agents; cytostatic agents for the treatment of cancer; diagnostic substances for use in nuclear medicine and radiation therapy; immunoactive and immunoreactive agents; and a number of other agents such as transmitters and their respective receptor-agonists and receptor-antagonists, their respective precursors or metabolites; antibiotics, antispasmodics, antihistamines, antinauseants, relaxants, stimulants, “sense” and “anti-sense” oligonucleotides, cerebral dilators, psychotropics, anti-manics, vascular dilators and constrictors, anti-hypertensives, migraine treatments, hypnotics, and anti-epileptics.

Typical active ingredients can be any substance affecting the nervous system or used for diagnostic tests of the nervous system. These are described by Gilman et al. (1990), “Goodman and Gilman's—The Pharmacological Basis of Therapeutics”, Pergamon Press, New York, and include the following agents:
acetylcholine and synthetic choline esters, naturally occurring cholinomimetic alkaloids and their synthetic congeners, anticholinesterase agents, ganglionic stimulants, atropine, scopolamine and related antimuscarinic drugs, catecholamines and sympathomimetic drugs, such as epinephrine, norepinephrine and dopamine, adrenergic agonists, adrenergic receptor antagonists, transmitters such as GABA, glycine, glutamate, acetylcholine, dopamine, 5-hydroxytryptamine, and histamine, neuroactive peptides; analgesics and anesthetics such as opioid analgesics and antagonists; preanesthetic and anesthetic medications such as benzodiazepines, barbiturates, antihistamines, phenothiazines and butylphenones; opioids; antiemetics; anticholinergic drugs such as atropine, scopolamine or glycopyrrolate; cocaine; chloral derivatives; ethchlorvynol; glutethimide; methyprylon; meprobamate; paraldehyde; disulfiram; morphine, fentanyl and naloxone;
centrally active antitussive agents;
psychiatric drugs such as phenothiazines, thioxanthenes and other heterocyclic compounds (e.g., halperiodol); tricyclic antidepressants such as desimipramine and imipramine; atypical antidepressants (e.g., fluoxetine and trazodone), monoamine oxidase inhibitors such as isocarboxazid; lithium salts; anxiolytics such as chlordiazepoxyd and diazepam;
anti-epileptics including hydantoins, anticonvulsant barbiturates, iminostilbines (such as carbamazepine), succinimides, valproic acid, oxazolidinediones and benzodiazepines;
anti-Parkinson drugs such as L-DOPA/CARBIDOPA, apomorphine, amantadine, ergolines, selegeline, ropinorole, bromocriptine mesylate and anticholinergic agents;
antispasticity agents such as baclofen, diazepam and dantrolene;
neuroprotective agents, such as excitatory amino acid antagonists, neurotrophic factors and brain derived neurotrophic factor, ciliary neurotrophic factor, or nerve growth factor; neurotrophin(NT) 3 (NT3); NT4 and NT5; gangliosides; neuroregenerative agents;
drugs for the treatment of addiction and drug abuse include opioid antagonists and anti-depressants;
autocoids and anti-inflammatory drugs such as histamine, bradykinin, kallidin and their respective agonists and antagonists;
chemotherapeutic agents including alkylating agents (e.g., cyclophosphamide, mechlorethamine, chlorambucil, melphalan, dacarbazine, temozolomide, nitrsoureas); anthracyclines (e.g., daunorubicin, doxorubicin, epirubicin, idarubicin, mitoxantrone, valrubicin); taxanes (e.g., paclitaxel; docetaxel; abraxane; taxotere) epothilones; histone deaceytlase inhibitors (e.g., vorinostat, romidepsin); topoisomerase I inhibitors (e.g., irinotecan, topotecan); topoisomerase II inhibitors (e.g., etoposide, teniposide, tafluposide); kinase inhibitors (e.g., bortezomib, erlotinib, geitinib, imatininb, vemurafenib, vismodegib); nucleotide analogs and precursor analogs (e.g., azacitidine, azathioprine, capecitabine, cytarabine, doxiflridine, fluorouracil, gemcitabine, hydroxyurea, mercaptopurine, methotrexate, tioguanine); peptide antibiotics (e.g., bleomycin, actinomycin); retinoids (e.g., tretinoin, alitretinoin, bexarotene); platinum-based agents (e.g., carboplatin, cisplatin, oxaliplatin); and vinca alkaloids and derivatives (e.g., vinblastine, vincristine, vindesine, vinorelbine);
anti-inflammatory drugs such as phenylbutazone, indomethacin, naproxen, ibuprofen, flurbiprofen, diclofenac, dexamethasone, prednisone and prednisolone;
cerebral vasodilators such as soloctidilum, vincamine, naftidrofuryl oxalate, co-dergocrine mesylate, cyclandelate, papaverine, and nicotinic acid;
anti-infective agents such as erythromycin stearate, and cephalexin.

Exemplary antibodies that can be transported into the brain, spinal cord, or other component of the CNS include antibodies that bind tau, β-amyloid, α-synuclein, TDP-43, CD20, IL-6R, a human complement protein (e.g., C5, C3, C1q), B-cell activating factor (BAFF), VEGF-A, CD25, alpha-4 integrin, and AQP4. In certain instances, the antibody's effector function is reduced or disabled (e.g., effector disable anti-AQP4 antibody). In certain embodiments, the antibody that is delivered to the brain, spinal cord, or other component of the CNS is a B cell depleting antibody (e.g., one that is useful for treating MS such as rituximab, ocrelizumab). In other embodiments, the antibody that is delivered to the brain, spinal cord, or other component of the CNS is a growth factor receptor blocker (e.g., trastuzumab) or a cytotoxic antibody for chemotherapy (antibodies coupled to cytotoxic agents such as those described in Ducry and Stump, Bioconjugate Chem., 21:5-13 (2010)).

Antibodies to tau are described in, e.g., US 2013/0295021 and US 2015/0344553 (both of which are incorporated by reference herein in their entireties); antibodies to β-amyloid are described in, e.g., US 2010/0202968 (incorporated by reference herein in its entirety); antibodies to a-synuclein are described in, e.g., US 2011/0300077 and US 2014/0369940 both of which are incorporated by reference herein in their entireties); antibodies to TDP-43 are described in, e.g., US 2014/0255304 (incorporated by reference herein in its entirety); antibodies to CD20 are described in e.g., U.S. Pat. Nos. 9,359,443; 9,296,821; 9,045,543; 8,057,793; 8,101,179; 7,727,525; 7,744,877; 7,422,739 (all of which are incorporated by reference herein in their entireties); and antibodies to IL-6R are described in e.g., U.S. Pat. Nos. 9,273,150; 8,753,634; 8,632,774; 9,017,678; 8,580,264 (all of which are incorporated by reference herein in their entireties).

Exemplary anti-tau antibodies are the antibodies 4E4 described in US 2013/0295021 and 6C5 and 40E8 described in US 2015/0344553. Exemplary anti-α-synuclein antibodies are the 12F4 antibody described in US 2011/0300077 antibody and the 21D11 antibody described in US 2014/0369940. An exemplary β-amyloid antibody is the 12F6A antibody described in US2010/0202968. Exemplary anti-TDP43 antibodies are the 41D1, 51C1, and 14W3 antibodies described in US 2014/0255304. An exemplary anti-BAFF antibody is belimumab; an exemplary anti-VEGF-A antibody is bevacizumab; an exemplary anti-CD20 antibodies are ocrelizumab, ofatumumab, and rituximab; an exemplary anti-IL-6R antibody is tocilizumab; an exemplary anti-CD25 antibody is daclizumab; an exemplary anti-alpha-4 integrin antibody is natalizumab; an exemplary anti-human C5 complement protein antibody is eculizumab; and an exemplary anti-AQP4 antibody is aquaporumab.

In certain embodiments, the methods disclosed herein enhance the delivery of an agent such as azathioprine, mycophenolate mofetil, methotrexate, hydroxychloroquine, corticosteroids, and cyclophosphamide, to a component of the CNS (e.g., brain, spinal cord) relative to delivery in the absence of administration of an anti-HSPA5 antibody.

The methods of this disclosure are particularly useful to transport any agent with poor access to the central nervous system. For example, poorly brain-penetrant small molecule compounds such as the chemotherapeutic agents gefitinib, lapitinib, erlotinib, vatalanib, imatinib, temsirolimus, and bortezomid; nitrosoureas such as carmustine, lomustine and nimustine; alkylating agents such as temozolomide; antineoplastic agents for the treatment of CNS tumors such as anthracyclines, platinum (II) complexes, paclitaxel, etoposide, irinotecan, topotecan, and methothrexate; EGFR inhibitors; protein kinase C-beta inhibitors; angiogenesis pathway inhibitors; PDGR inhibitors; and mTOR inhibitors, can be delivered to the brain, spinal cord, or other component of the CNS using the methods disclosed herein. In certain instances, the methods of this disclosure provide for the enhanced introduction of such agents into the brain relative to delivery in the absence of administration of an anti-HSPA5 antibody.

### Methods of Use

Provided herein are methods to identify if a human subject is at risk of relapse of NMO. In certain embodiments, the human subject may be on medication to control NMO, or may no longer be taking medication for NMO. The method involves measuring anti-HSPA5 titers in a biological sample (e.g., blood) from the subject. If the levels of anti-HSPA5 antibody are higher than a control level, then the subject is determined as having a risk of relapse of NMO. The “control level” can be the titer of anti-HSPA5 antibodies in a subject who is determined not to have NMO, or a prior titer determined in that same subject during a time of NMO disease quiescence, or an antibody titer previously determined to be the anti-HSPA5 antibody titer in normal subjects (i.e., subjects who do not have NMO).

Also featured are methods to assess the severity of an attack of NMO in a human subject. The method involves measuring anti-HSPA5 titers in a biological sample (e.g., blood) from the subject during an NMO attack and comparing these levels to an attack severity scale. The attack severity can be established using the scale used in the eculizumab study optic-spinal impairment scale in the Appendix to S. J. Pittock et al., Lancet Neurol., 12(6):554-562 (2013), incorporated by reference herein in its entirety). In an alternative embodiment, the method involves measuring anti-HSPA5 titers in a biological sample (e.g., blood) from the subject during an NMO attack and comparing these levels to controls. The controls can include anti-HSPA5 antibody titers from subjects who do not have NMO; titers from subjects with mild NMO; titers from subjects with moderate NMO; and titers from subjects with severe NMO. “Mild” corresponds to Grade 1 of the CTCAE v4.0 and means asymptomatic or mild symptoms; clinical or diagnostic observations only; intervention not indicated. “Moderate” corresponds to Grade 2 of the CTCAE v4.0 and means moderate; minimal, local or noninvasive intervention indicated. “Severe” corresponds to Grade 3 of the CTCAE v4.0 and means severe or medically significant but not immediately life-threatening; hospitalization or prolongation of hospitalization indicated; disabling. If the levels of anti-HSPA5 antibody are comparable to the control corresponding to subjects who do not have NMO, then the subject is identified as not likely having a true attack of NMO. If the levels of anti-HSPA5 antibody are comparable to the control corresponding to mild NMO, then the subject is identified as having a risk of developing mild attack severity. If the levels of anti-HSPA5 antibody are comparable to the control corresponding to moderate NMO, then the subject is identified as having a risk of developing an attack of moderate severity. If the levels of anti-HSPA5 antibody are comparable to the control corresponding to severe NMO, then the subject is identified as having a risk of developing an NMO attack of high severity.

The disclosure also provides methods of determining when an NMO attack is imminent. The method involves measuring anti-HSPA5 titers in a biological sample (e.g., blood) from the subject and comparing these levels to a control level. The “control level” can be the titer of anti-HSPA5 antibodies in a subject who is determined to have NMO, or an antibody titer previously determined to be the anti-HSPA5 antibody titer in subjects who have developed NMO, or a prior titer determined in that same subject at a time of NMO disease quiescence. If the levels are similar or higher than the control level, the subject is determined to be at high risk for an imminent NMO attack.

The anti-HSPA5 antibodies described herein can be used to diagnose a human subject with neuropsychiatric systemic lupus erythematosus (NP-SLE). The method involves measuring anti-HSPA5 titers in a biological sample (e.g., blood) from the subject. If the levels of anti-HSPA5 antibody are higher than a control level, then the subject is diagnosed as having or likely to develop NP-SLE. In this context, the “control level” can be the titer of anti-HSPA5 antibodies in a subject who is determined not to have NP-SLE or an antibody titer previously determined to be the anti-HSPA5 antibody titer in normal subjects (i.e., subjects who do not have NP-SLE).

The anti-HSPA5 antibodies described herein can also be used to identify if a human subject is at risk of relapse of NP-SLE. Such a subject may be on medication to control NP-SLE or may no longer be taking medication for NP-SLE. The method involves measuring anti-HSPA5 titers in a biological sample (e.g., blood) from the subject. If the levels of anti-HSPA5 antibody are higher than a control level, then the subject is determined as having a risk of relapse of NP-SLE. The “control level” can be the titer of anti-HSPA5 antibodies in a subject who is determined not to have NP-SLE or an antibody titer previously determined to be the anti-HSPA5 antibody titer in normal subjects (i.e., subjects who do not have NP-SLE) or a prior titer determined in that same subject at a time of NP-SLE disease quiescence.

The anti-HSPA5 antibodies described herein can also be used to assess the severity of an attack of NP-SLE in a human subject. The method involves measuring anti-HSPA5 titers in a biological sample (e.g., blood) from the subject and comparing these levels to controls. The controls can include anti-HSPA5 antibody titers from subjects who do not have NP-SLE; patients with mild NP-SLE; patients with moderate NP-SLE; and patients with severe NP-SLE. If the levels of anti-HSPA5 antibody are comparable to the control corresponding to subjects who do not have NP-SLE, then the subject is identified as not having a risk of developing an attack of NP-SLE. If the levels of anti-HSPA5 antibody are comparable to the control corresponding to mild NP-SLE, then the subject is identified as having a risk of developing a mild attack of NP-SLE. If the levels of anti-HSPA5 antibody are comparable to the control corresponding to moderate NP-SLE, then the subject is identified as having a risk of developing a moderate attack of NP-SLE. If the levels of anti-HSPA5 antibody are comparable to the control corresponding to severe NP-SLE, then the subject is identified as having a risk of developing a severe attack of NP-SLE.

The disclosure also provides methods of determining when an NP-SLE attack is imminent. The method involves measuring anti-HSPA5 titers in a biological sample (e.g., blood) from the subject and comparing these levels to a control level. The “control level” can be the titer of anti-HSPA5 antibodies in a subject who is determined to have NP-SLE, or an antibody titer previously determined to be the anti-HSPA5 antibody titer in subjects who have developed NP-SLE, or a prior titer determined in that same subject at a time of NP-SLE disease quiescence. If the levels are similar or higher than the control level, the subject is determined to be at high risk for an imminent NP-SLE attack.

### Methods of Administration

The anti-HSPA5 antibody or antigen-binding fragment thereof described herein can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody or antigen-binding fragment thereof can also be tailored for the individual case, e.g., by monitoring the subject, e.g., using tomographic imaging, e.g., to visualize a tumor.

The antibody or antigen-binding fragment thereof can be administered as a fixed dose, or in a mg/kg dose. Dosage regimens are adjusted to provide the desired response, e.g., a therapeutic response or a combinatorial therapeutic effect. Generally, doses of the anti-HSPA5 antibody (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In specific embodiments, a subject in need of treatment with an anti-HSPA5 antibody is administered the antibody at a dose 2 mg/kg, 4 mg/kg, 5 mg/kg, 10 mg/kg, 15 mg/kg, 20 mg/kg, 30 mg/kg, 35 mg/kg, or 40 mg/kg.

A composition may comprise about 1 mg/mL to 100 mg/ml or about 10 mg/mL to 100 mg/ml or about 50 to 250 mg/mL or about 100 to 150 mg/ml or about 100 to 250 mg/ml of anti-HSPA5 antibody or antigen-binding fragment thereof.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

An anti-HSPA5 antibody or antigen-binding fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. In one embodiment, the anti-HSPA5 antibody or antigen-binding fragment thereof described herein is administered biweekly. In a specific embodiment, the anti-HSPA5 antibody or antigen-binding fragment thereof described herein is administered monthly. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

In certain embodiments, the anti-HSPA5 antibody or antigen-binding fragment thereof is administered subcutaneously at a concentration of about 1 mg/mL to about 300 mg/mL (e.g., 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 mg/mL, 250 mg/mL). In one embodiment, the anti-HSPA5 antibody or antigen-binding fragment thereof is administered subcutaneously at a concentration of 50 mg/mL. In a different embodiment, the anti-HSPA5 antibody or antigen-binding fragment thereof is administered subcutaneously at a concentration of 150 mg/mL. In another embodiment, the anti-HSPA5 antibody or antigen-binding fragment thereof is administered subcutaneously at a concentration of 200 mg/mL. In another embodiment, the anti-HSPA5 antibody or antigen-binding fragment thereof is administered intravenously at a concentration of about 1 mg/mL to about 300 mg/mL (e.g., 1 mg/mL, 5 mg/mL, 10 mg/mL, 25 mg/mL, 50 mg/mL, 75 mg/mL, 100 mg/mL, 125 mg/mL, 150 mg/mL, 175 mg/mL, 200 mg/mL, 250 mg/mL). In a particular embodiment, the anti-HSPA5 antibody or antigen-binding fragment thereof is administered intravenously at a concentration of 50 mg/mL. In one embodiment, the anti-HSPA5 antibody or antigen-binding fragment thereof is administered intravenously at a concentration of 75 mg/mL. The administration can be e.g., every 2-3 days, weekly, biweekly, or monthly.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1

### Non-AQP4 Antibodies in NMO-IgG Cause BBB Dysfunction

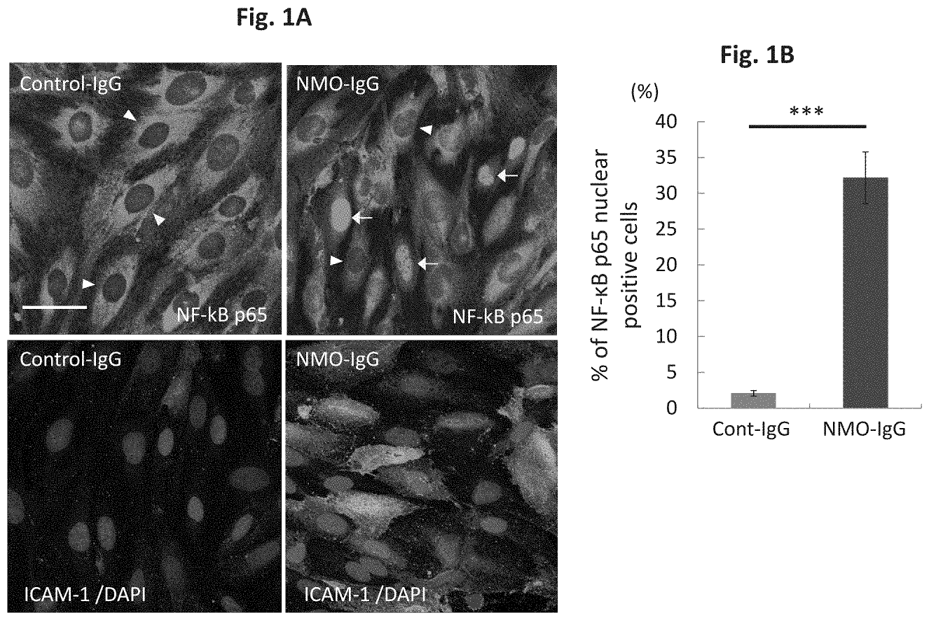

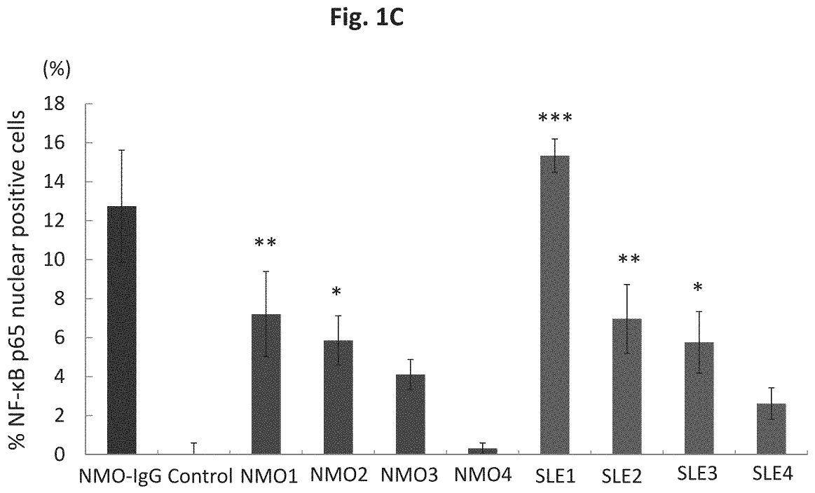

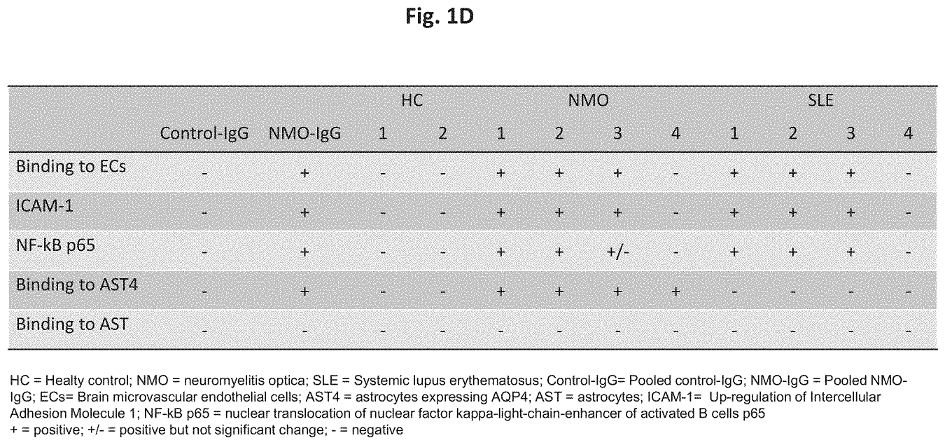

To analyze the effect of Neuromyelitis optica (NMO) sera on blood-brain barrier (BBB)-endothelial cells, IgG pooled from 50 NMO patients (NMO-IgG) or pooled control-IgG was incubated with human BMEC TY-10 cells in monolayer culture and immunohistochemical analysis was performed for NF-κB p65 localization after 1 hour and ICAM-1 expression after 24 h. Pooled NMO-IgG induced nuclear translocation of NF-κB p65 and upregulation of ICAM-1 in BMECs, and pooled control-IgG was inert in these assays (FIGS. 1A, and 1B).

Because NMO-IgG contained antibodies derived from multiple individuals, it remained uncertain whether NMO-derived antibodies from individual cases could mediate these effects or if complementary effects of multiple antibodies were acting in concert to induce inflammatory changes in BMECs. To address this question, 10 IgG preparations from individual NMO cases, inflammatory controls, and healthy controls (4 seropositive NMO; 4 AQP4-antibody negative SLE; 2 healthy controls) were assayed. IgG from 2/4 NMO and 3/4 SLE cases bound BMECs and elicited ICAM-1 upregulation and NF-κB p65 nuclear translocation (FIGS. 1C and 1D), demonstrating that individual patient serum IgG could bind and activate BBB endothelium. This activity was not specific for NMO cases but was also present in SLE. AQP4-IgG was present in each of the NMO cases, but was not observed in the SLE or healthy controls (FIG. 1D) indicating that AQP4-IgG was not required for binding and activation of BMECs.

### Example 2

### Recombinant Antibodies Isolated from NMO Patients Bind and Activate BMECs

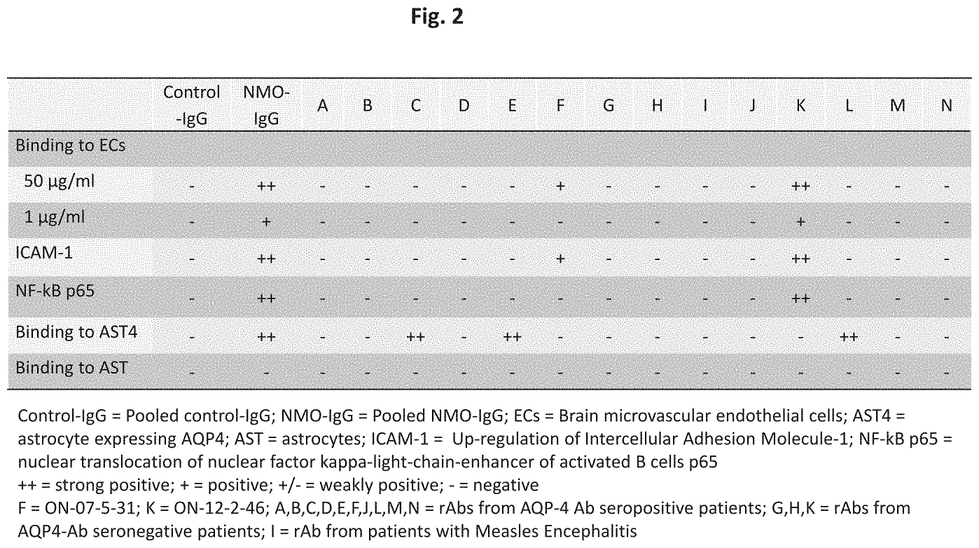

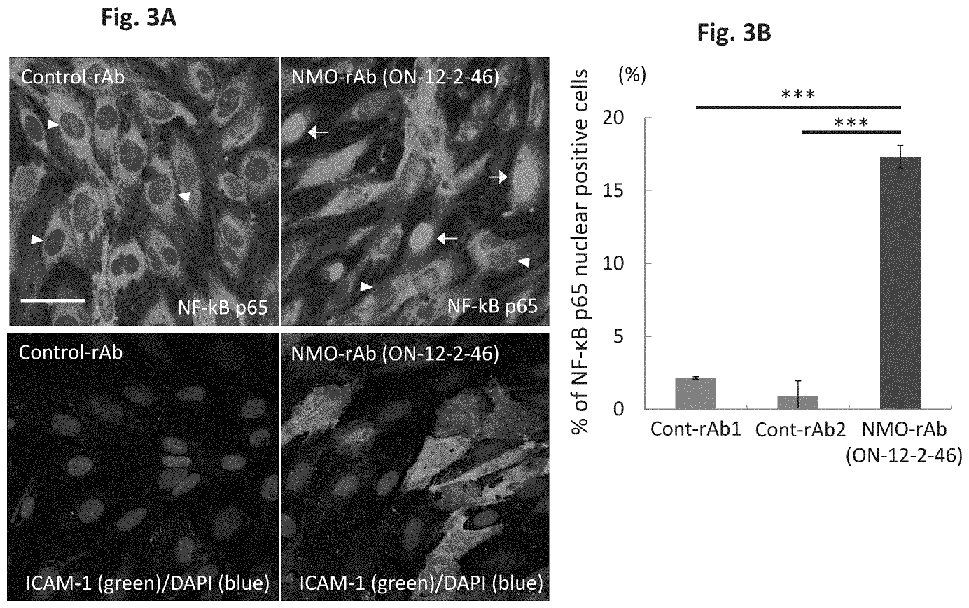

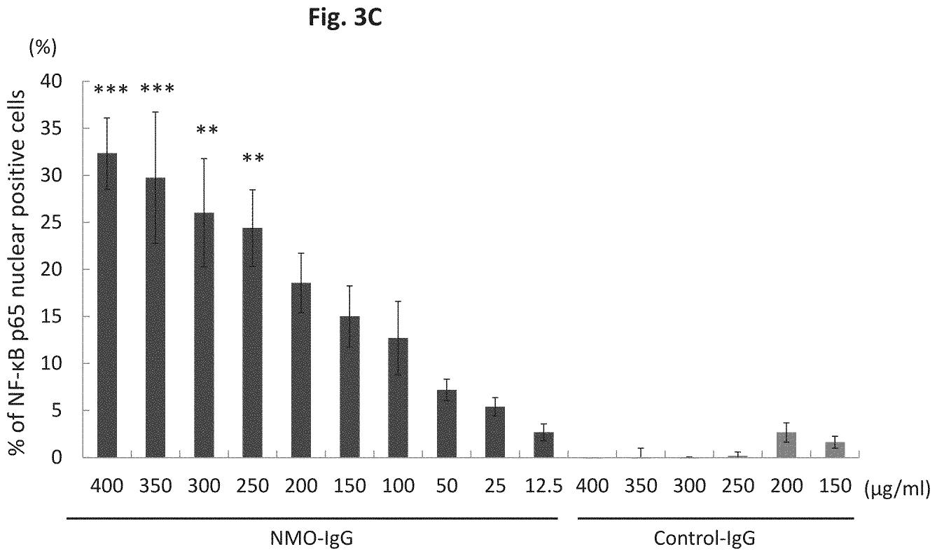

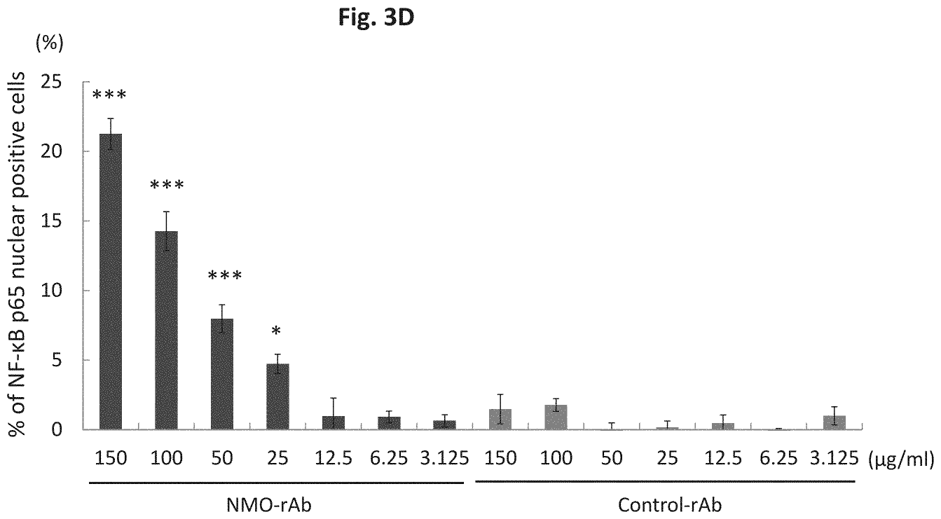

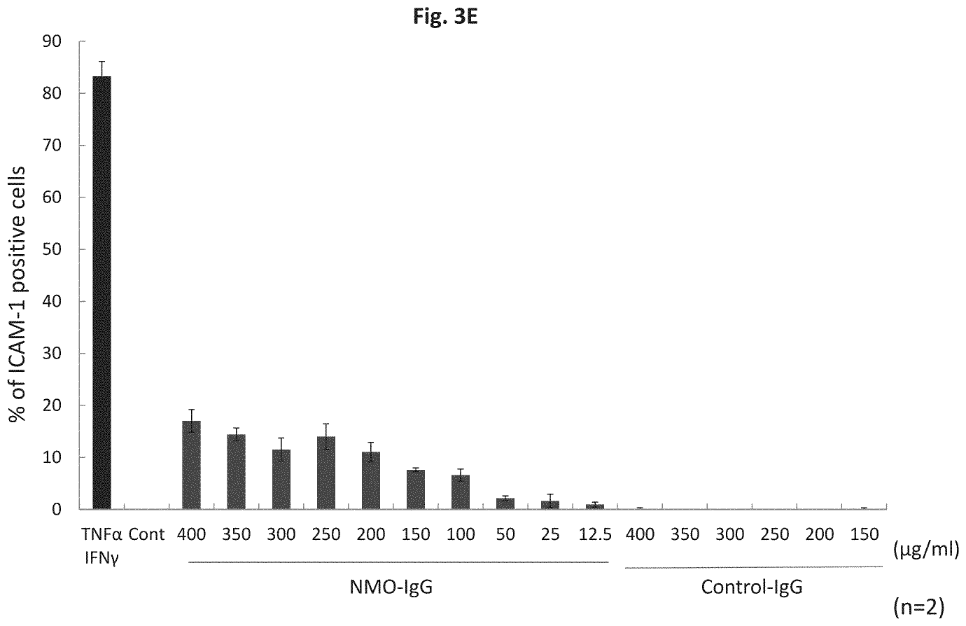

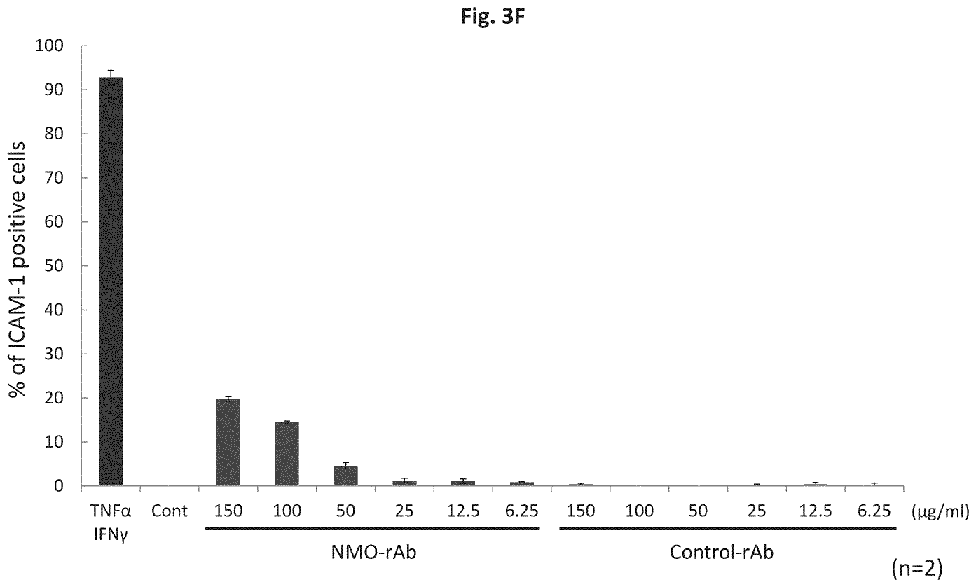

It remained possible that multiple BMEC specificities might be present in antibodies from a single patient. To address the question whether a monoclonal antibody from the NMO B-cell repertoire could bind and activate BMECs, a masked assay of a panel of AQP4-reactive and non-reactive recombinant antibodies (rAbs) prepared from NMO and control CNS plasmablasts (13 NMO-rAbs and 1 control-rAb) was performed. Two NMO-rAbs (ON-12-2-46 and ON-07-5-31) strongly bound to BMECs at 50 μg/ml (FIG. 2). Both of these rAbs induced upregulation of ICAM-1 (data not shown); however, only ON-12-2-46 induced nuclear translocation of NF-κB p65 (FIGS. 3A and 3B). Neither ON-07-5-31 nor ON-12-2-46 bound to AQP4, consistent with previous experiments demonstrating that the effects of NMO-IgG on BMECs were independent AQP4-IgG. Furthermore, Western immunoblot analysis of endothelial cell line lysates using rAbs ON-12-2-46 and ON-07-5-31 detected a dominant band of identical electrophoretic mobility, suggesting that these two rAbs might target a single protein antigen (data not shown). The effects of both pooled NMO-IgG and non-AQP4 NMO-rAb (ON-12-2-46) on NF-κB p65 nuclear translocation and ICAM-1 upregulation in BMECs were dose-dependent (FIGS. 3C, 3D, 3E, and 3F).

To address whether the binding of NMO-rAb (ON-12-2-46) was common for microvascular endothelial cells (MECs) or relatively specific for BBB endothelial cells, the binding of ON-12-2-46 to MECs of distinct origins: BMECs (TY10), commercial human umbilical vein endothelial cells (HUVECs), human dermal MECs, and primary human kidney or lung MECs, was evaluated. The binding of rAb and ICAM-1 up-regulation following exposure to NMO-rAb were observed only in BMECs (data not shown).

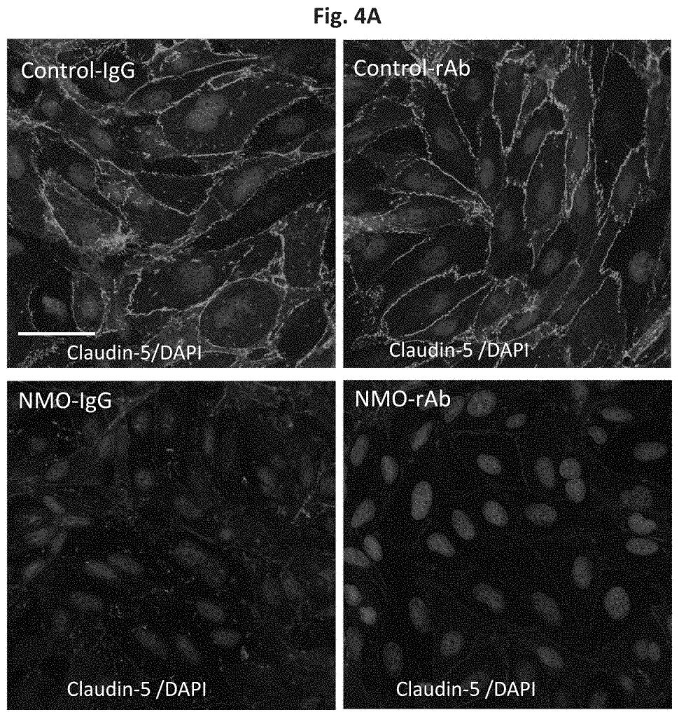

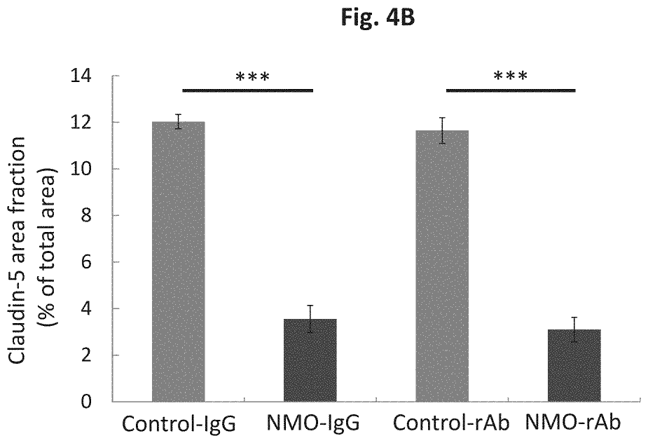

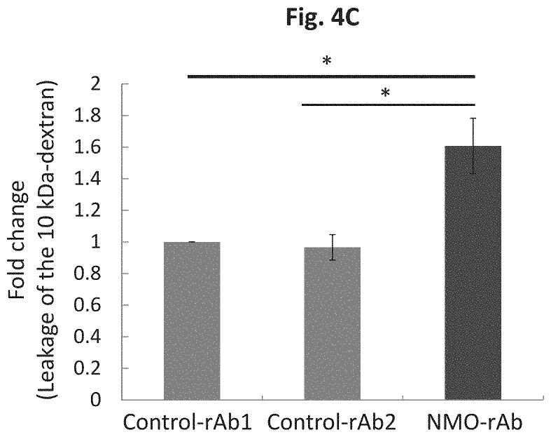

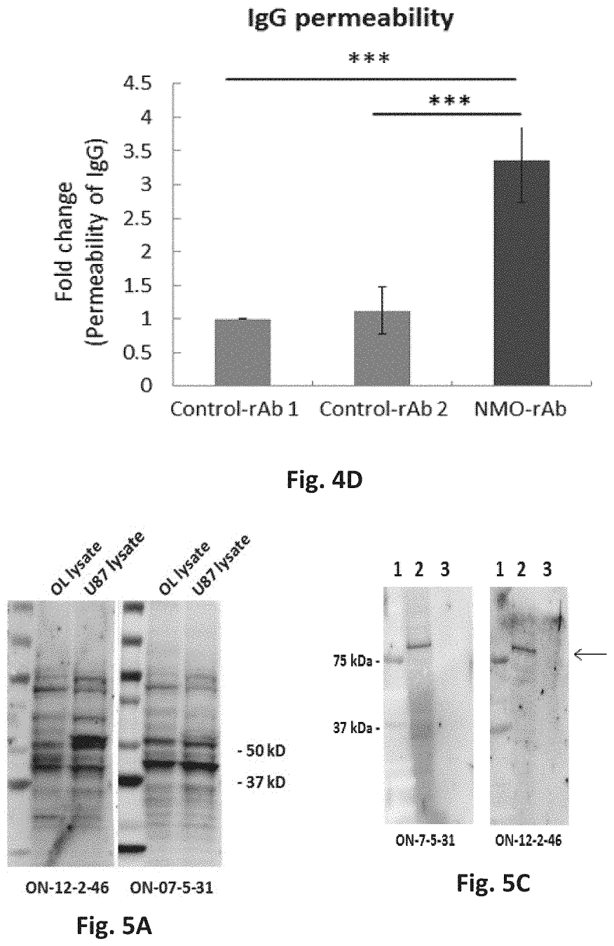

To evaluate whether BMEC activation mediated by NMO-rAb ON-12-2-46 was associated with structural change of tight junctions and BBB permeability, claudin-5 immunoreactivity and the permeability constant for 10 kDa-dextran was examined. The area fraction of claudin-5 expression in BMECs was significantly decreased after exposure to pooled NMO-IgG or rAb ON-12-2-46 but not control-IgG or control-rAbs (FIGS. 4A and 4B). The permeability constant of 10kDa-dextran and IgG were significantly increased after incubation with rAb ON-12-2-46, but not 2 control-rAbs (FIGS. 4C and 4D), indicating that rAb ON-12-2-46 could enhance BBB permeability and alter tight junctions.

### Example 3

### Identification of GRP78/HSPA5 as a Target in NMO

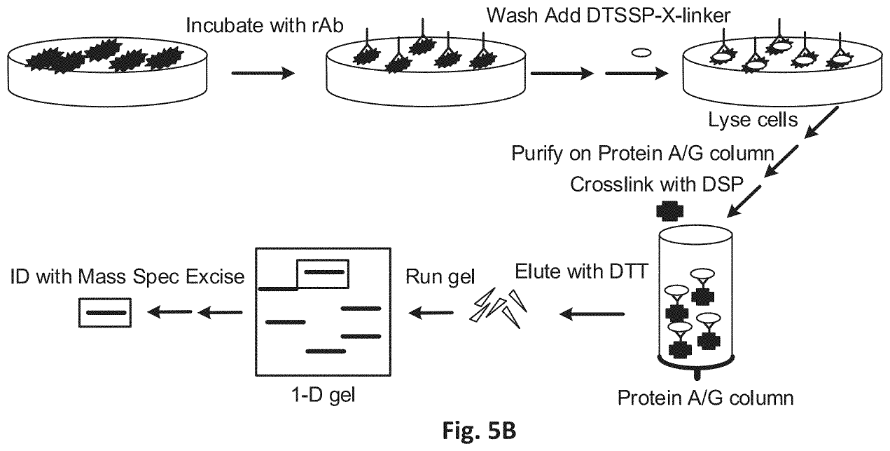

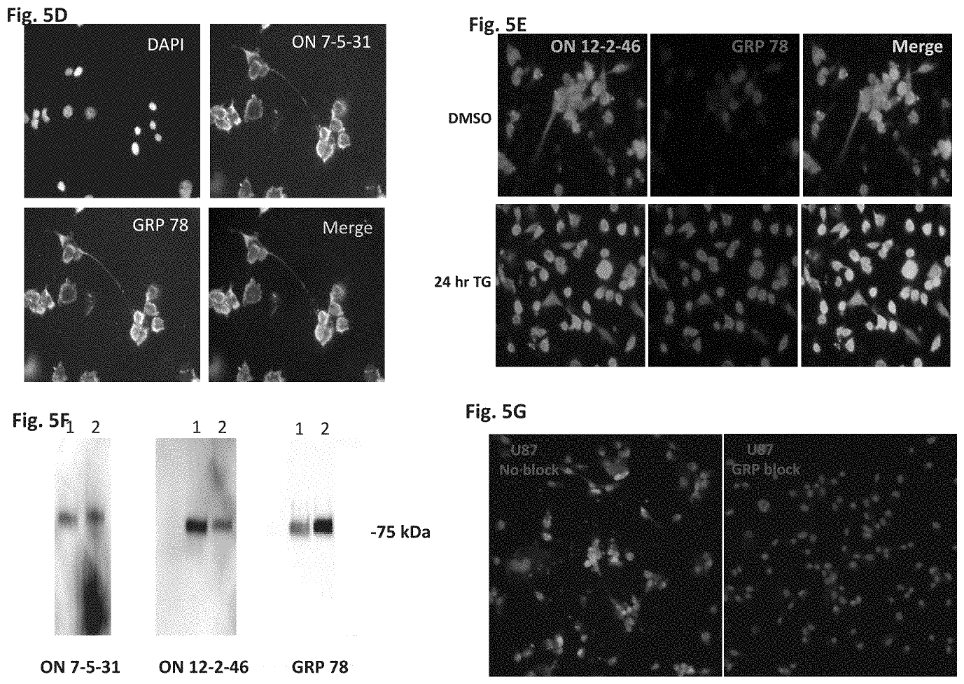

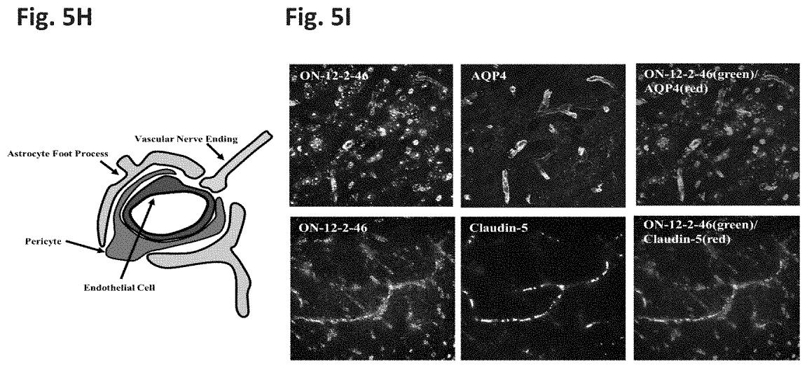

Proteomics was then deployed to identify the antigenic targets of NMO-rAbs ON-12-2-46 and ON-07-5-31, the only two rAbs binding BMECs. Both rAbs bound to the membrane of multiple cell lines, including fixed U87MG cells and live OL cells. In addition, both rAbs bound to both neuronal and glial cells in mouse brain sections as well as choroid plexus (data not shown). Western blot analyses of crude membrane fraction from U87MG and OL cells produced multiple bands of similar molecular weight (FIG. 5A). To reduce background and enhance specificity, NMO rAbs ON-12-2-46 and ON-7-5-31 were reversibly crosslinked to their cell surface antigens on U87MG cells. After solubilization and purification on protein A/G beads, the antigenic targets were released with DTT and analyzed by Western blot. Both ON-12-2-46 and ON-7-5-31 detected a single protein band of molecular weight of 75 kDa (FIGS. 5B and 5C) that was excised and analyzed by mass spectrometry.

Glucose-Regulated protein 78 (GRP78) also known as HSPA5 was identified as the target antigen of both ON-07-5-31 and ON-12-2-46 with 41 and 51 tryptic peptides respectively, covering 38% and 40% of the GRP78/HSPA5 protein. Double immunostaining with commercial anti-GRP78 antibodies and both rAbs demonstrated co-localization in both cell lines (FIGS. 5D and 5E). Pre-incubating recombinant GRP78 protein with the NMO-rAbs decreased binding to U87MG cells (FIG. 5G). Immunoblot analysis demonstrated that both rAbs reacted with recombinant GRP78 protein (FIG. 5F). Isolation of two rAbs from two different NMO patients with a single specificity suggested that GRP78/HSPA5 represents a NMO autoantibody targeted to endothelium. Staining of non-permeabilized mouse brain tissue with GRP78-specific NMO-rAb ON-12-2-46, AQP4, and claudin-5 demonstrates that GRP78 staining co-localizes with claudin-5 on the surface of endothelial cells internal to astrocytic AQP4 (FIGS. 5H and 5I).

### Example 4

### Depletion of GRP78 Antibodies from NMO-IgG Decreases the Biological Effect on BMECs

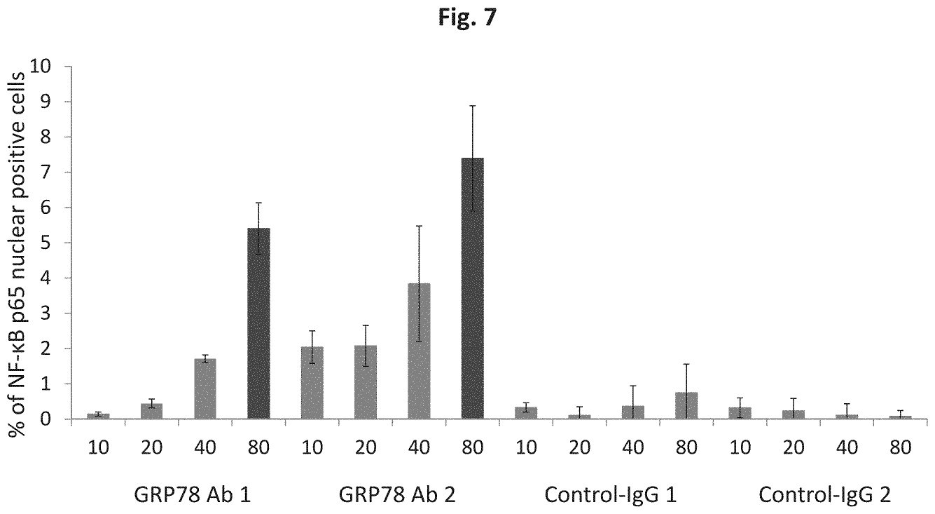

Abundant cell-cell boundary expression of GRP78 in fixed or living BMECs was detected using commercial GRP78 antibodies, as compared to that in HUVECs or MECs from kidney and dermis (data not shown). We also observed that 2 commercial GRP78 antibodies induced nuclear translocation of NF-κB p65 (FIG. 7).

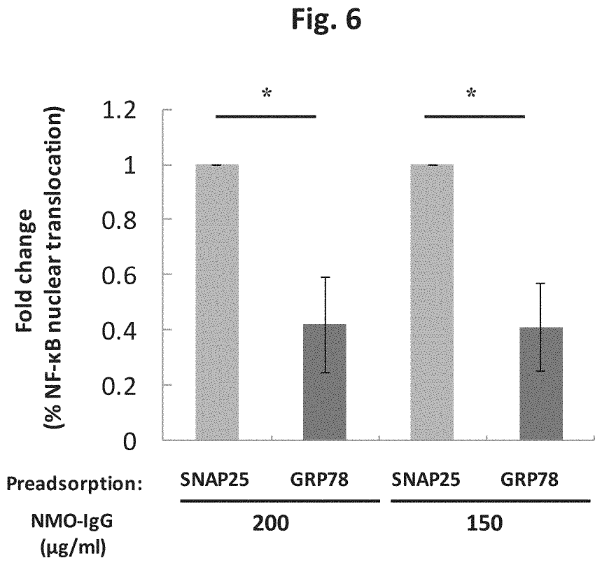

Although NMO-rAb, ON-12-2-46, was sufficient to mediate effects on BMECs in vitro comparable to bulk, pooled NMO-IgG, it remained unclear whether GRP78 antibodies were present in bulk pooled NMO-IgG, and, if so, were necessary for NF-κB p65 nuclear translocation. Using cell lysates from cells that overexpressed FLAG-GRP78 or control FLAG-SNAP25, immunoadsorption of anti-GRP78 antibodies from pooled NMO-IgG was performed. NMO-IgG specifically depleted of anti-GRP78 antibodies showed significant reduction in NF-κB p65 nuclear translocation in BMECs (FIG. 6).

### Example 5

### Administration of GRP78-rAb Causes Localized Extravasation of Albumin, Fibrinogen, and IgG into Brain Tissue

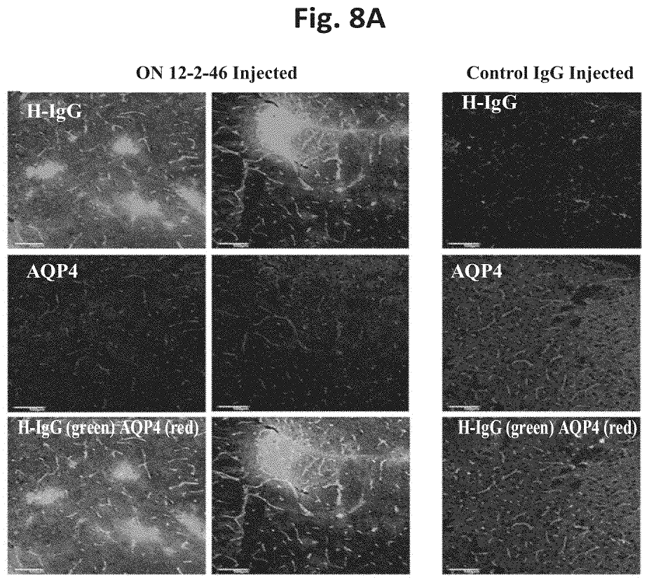

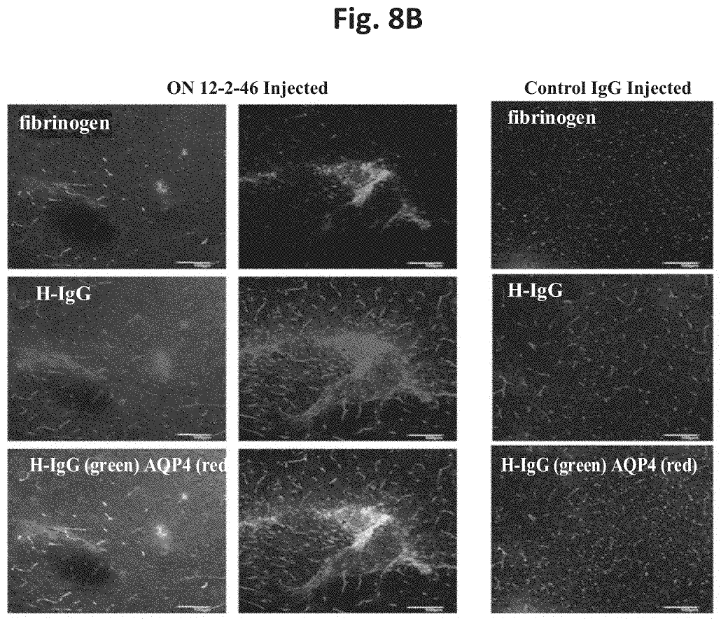

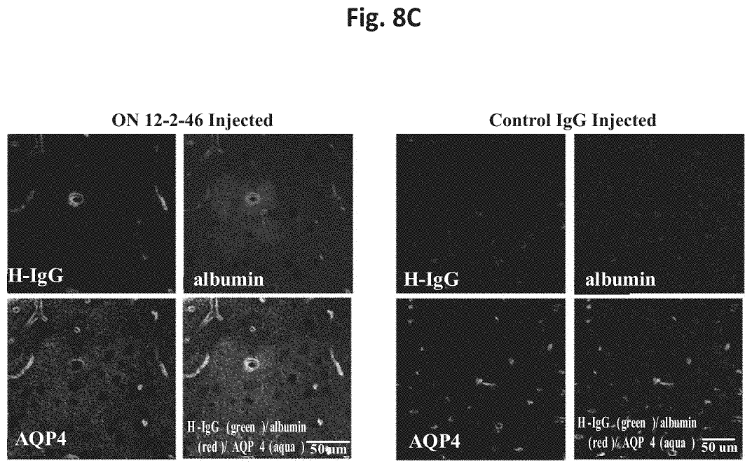

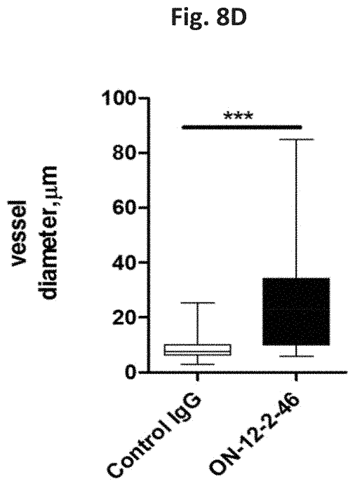

To examine whether administration of GRP78 rAb ON-12-2-46 caused brain vascular permeability in vivo, either murinized rAb ON-12-2-46 or isotype control rAb was administered to mice (intravenously or intraperitoneally) daily for 7 days in conjunction with human AQP4-specifc rAb ON-7-5-53 (beginning on day 3). Two days after the final administration of rAb, the animals were sacrificed, and the non-permeabilized brain tissue examined for leakage of plasma components (injected human IgG, endogenous fibrinogen, and albumin) into the extravascular space (FIGS. 8A-C). Mice treated with isotype control rAb showed no evidence of vascular leakage (FIGS. 8A-C), whereas mice treated with GRP78 rAb ON-12-2-46 showed discrete foci of leakage of human IgG (FIGS. 8A and B), fibrinogen (FIG. 8B), and albumin (FIG. 8C) that was independent of the amount of rAb delivered over the dose range (16-20 mg/kg). In addition, mice treated with GRP78-specific rAb demonstrated abnormally large diameter blood vessels (FIG. 8D), suggesting either loosened cell-cell endothelial contacts or vasodilatory reaction to GRP78 engagement by rAb.

### Materials & Methods Employed in the Above Examples:

### Patient Samples

NMO-IgG were prepared from pooled waste plasma (50 NMO patients who received therapeutic plasmapheresis) and control-IgG from pooled sera of healthy individuals (Department of Laboratory Medicine and Pathology, Mayo Clinic, Rochester, Minn., USA). A total of 10 IgG preparations from individual cases (4 NMO patients, 4 SLE patients and 2 healthy volunteers) were analyzed (Department of Laboratory Medicine and Pathology, Mayo Clinic, Rochester, Minn., USA). IgG preparations were purified by protein G affinity and adjusted for functional assays by extensive dialysis.

Construction, Expression, and Purification of rAbs

Monoclonal recombinant antibodies (rAbs) were generated from 7 AQP4 seropositive and seronegative patients and an idiopathic meningitis control. Single cell analysis of B cells, antibody production and AQP4-positive/negative CSF IgG variable region heavy- (VH) and light-chain (VL) sequences were recovered from CD19-CD138+ plasmablasts by single cell fluorescent-activated cell sorting (FACS), reverse transcriptase PCR (RT-PCR), and DNA sequencing as described previously (Bennett J L et al, Ann Neurol., 66: 617-29 (2009)). Recombinant antibodies were produced in HEK293 cells (Invitrogen, Carlsbad, Calif., USA; R620-07) as described previously (Bennett J L et al, 2009, supra). Human IgG1 rAbs generated from a patient with chronic meningitis (IC05-2-2) (Owens G P et al, Ann Neurol., 65: 639-49 (2009)) or a measles virus-specific rAb (2B4) serve as isotype-matched controls (Burgoon M P et al, J Neuroimmunol., 94:204-11 (1999)).

### Cell Culture and Treatment

BMECs are adult human brain microvascular endothelial cells immortalized with temperature sensitive (ts) SV40-LTA as previously described (24. Sano Y et al, J Cell Physiol., 225: 519-28 (2010)). BMECs were grown in MCDB131 media (Sigma Aldrich) supplemented with EGM-2 SingleQuot Kit Supply and Growth Factors (Lonza), 20% FBS. Human umbilical vein endothelial cells (HUVECs) and microvascular endothelial cells (MECs) from derma were purchased from Lonza. Primary MECs from kidney or lung were isolated from human samples as previously described (Ligresti G, et al, J Am Soc Nephrol., 11. pii: ASN.2015070747 (2015)). Human astrocytes (AST) and astrocytes transfected to express AQP4 M23 (AST-A4) were grown in Astrocyte Media (AM, ScienCell) containing 2% heat-inactivated fetal bovine serum, astrocyte growth supplement and penicillin/streptomycin solution (ScienCell), as previously reported (Haruki H et al, Neurol Sci., 331(1-2):136-44 (2013)). AST-A4, but not AST, expressed cell-surface AQP4, although both cells expressed GFAP (Haruki H et al, 2013, supra). Cells were cultured in a humidified atmosphere of 5% carbon dioxide/95% air at 33° C. Analyses on BMECs were performed two days after a temperature shift to 37° C. Before analyses, cells were incubated with serum-free MCDB131 media for 24 hours. Cultured cells were treated with 400 μg/ml of pooled NMO-IgG, pooled control-IgG, IgG from individual patients, or 50 μg/ml of rAb for an hour (NF-κB p65 and GRP78 analyses) or 24 hours (ICAM-1 and claudin-5 analyses). A total of 14 rAbs were used for the pilot study, including AQP4 and non-AQP4 specific rAb from NMO patients or other disease controls (13 rAbs from the CSF of NMO patients and 1 rAb from the brain of a patient with measles encephalitis), which were randomly assigned from A to N for the purpose of blinded investigation. Cells treated with TNF-α (10 U/ml) or TNF-α (10 U/ml)/IFN-γ (5 U/ml) served as positive controls for NF-κB p65, ICAM-1 or claudin-5 expression.

### Antibodies for Immunohistochemistry

Primary antibodies used for immunohistochemistry were NF-κB p65 rabbit mAb (1:400, Cell Signaling Technology (CST), #8242), ICAM-1 mouse mAb (1:400, Santa Cruz, sc-18908), Claudin-5 rabbit mAb (1:1000, Abcam, ab53765) or mouse mAb (1:50, Life Technologies, #187364), GRP78 mouse monoclonal Ab (1:100, Santa Cruz, sc-376768) or rabbit polyclonal (1:100, Santa Cruz, sc-13968), albumin goat polyclonal (1:60, Abcam, ab19194), rabbit polyclonal FITC-fibrinogen (1:60, Dako, 2022-01), AQP4 rabbit polyclonal (1:200, Santa Cruz, sc-20812), VE-cadherin rabbit polyclonal Ab (1:1000, Abcam, ab33168) and PECAM mouse monoclonal Ab (1:100, R&D, BBA7). Secondary antibodies used for immunohistochemistry detection were Alexa Fluor® 488 goat anti-rabbit IgG, Alexa Fluor® 488 goat anti-human IgG and Alexa Fluor® 594 goat anti-rabbit IgG or Alexa Fluor® 594 goat anti-mouse IgG, Alexa Fluor® 594 donkey anti-goat, Alexa Fluor® 488 donkey anti-human, or Alexa Fluor® 647 donkey anti-rabbit (1:400, Life Technologies). Pooled IgGs, individual patient's IgGs and rAbs were also used as primary antibodies to detect the binding of IgG to cells.

### Cell Lines and Immunohistochemistry

BMECs were either fixed with 4% paraformaldehyde (NF-κB p65, GRP78, ICAM-1 and VE-cadherin staining) or 100% ethanol (claudin-5 staining), washed, then permeabilized with 0.3% Triton-X (NF-κB p65, GRP78, claudin-5 and VE-cadherin). Cells were then blocked overnight in 5% goat serum in PBS (ICAM-1) or 5% goat serum/0.3% Triton-X in PBS (NF-κB p65, claudin-5, GRP78 and VE-cadherin). Cells were incubated with primary antibodies for 2 hours at room temperature, followed by species-specific Alexa Fluor® secondary antibodies (1:400). To assay the binding of NMO-IgG/NMO-rAb to the cells, fixed BMECs were incubated for an hour at room temperature with 1 μg/ml of pooled-IgG or either 1 μg/ml or 50 μg/ml of rAbs as the primary antibody. Live AST or AST-A4 cells were incubated for an hour at 37° C. with 20 μg/ml of rAb as primary antibody, followed by incubation for 1 hour with goat anti-human IgG Alexa Fluor® 488 as the secondary antibody. Slides were mounted with ProLong® Gold antifade reagent (Life Technologies). Nuclei were stained with DAPI or Hoechst 33342 (Life Technologies). Images were taken on LSM780 confocal microscope operated by ZEN software version 2010 (Carl Zeiss) or a Leica DM2500 micro-scope (Leica Microsystems, Exton, Pa.). 20× air, 40× oil or 60× oil objectives were used to capture all images with a digital zoom factor of 2-4×. All image parameters remained constant (pinhole, power and gain) in order to capture images from independent experiments in a comparable manner. Noise was removed after deletion of non-specific signal and binary images were created automatically. The area fraction (percentage of claudin-5 positive area per total window area) was calculated using ImageJ software (NIH).

The U-87MG glioblastoma (U87MG) and human oligodendrocyte (OL) cell lines (Jordan I et al., J Virol 1999; 73: 7903-6 (1999)) were cultured on poly-ornithine-coated glass coverslips for 24-48 hours in MEM alpha or DMEM with glutamax containing 10% fetal calf serum (FCS), 1 mM sodium pyruvate, and 1× non-essential amino acids. Cells were fixed with 4% paraformaldehyde and stored at −20° C. until staining. For some experiments, U87MG cells were treated with 1 mM thapsigargin (Sigma Aldrich) overnight in the media noted above. For immunostaining, cells were rehydrated in phosphate-buffered saline (PBS) followed by blocking for an hour in PBS containing 4% normal goat sera with 0.3% Triton-X100. Primary antibodies (rAb: 10-20 μg/mL) were applied for 16 hours at 4° C. in blocking buffer, washed four times in PBS, incubated with goat anti-human Alexa Fluor 488 secondary antibodies (1:500) in blocking buffer for 2 hours, washed multiple times in PBS, and coverslips mounted using Prolong® Gold plus DAPI.

### Animal Injections, Tissue Preparation and Immunohistochemistry

Adult C57bl/6 mice were administered murinized NMO rAb ON-12-2-46 or isotype control rAb (2B4 or ICO-5-2-2) in combination with NMO AQP4-specific rAb ON-7-5-53 to induce BBB permeability. Two experimental paradigms were employed: 1) 16 mg/kg ON-12-2-46 or isotype control rAb delivered daily for 7 days (IV on days 1, 3, 5, 7 and IP on days 2, 4, 6) in conjunction with 5 mg/kg NMO human rAb ON-7-5-53 IV on days 3, 5, and 7; and 2) 20 mg/kg ON-12-2-46 or isotype control rAb delivered daily for 7 days (IV on days 1, 3, 5 and IP on days 2, 4, 6, 7) in conjunction with an escalating dose of NMO human rAb ON-7-5-53 (4-10 mg/kg) on days 3-7 (delivered IV or IP in combination with the murinized rAb). For each paradigm, two animals were treated with isotype control rAb and 3 animals were treated with NMO rAb ON-12-2-46. Two days after the last injection, the animals were deeply anesthetized and perfused with 3 mM EDTA in PBS, followed by 4% paraformaldehyde in PBS. Brains were removed, postfixed overnight in 4% paraformaldehyde, and cryoprotected overnight in 20% sucrose and then overnight in 30% sucrose, all at 4° C. Cryostat sections (20 μm) of tissue embedded in optimal cutting temperature (OCT) freezing media were collected on SuperFrost Plus microscope slides (Fisher Scientific, Pittsburgh, Pa., USA), then stored at −80° C. Prior to immunostaining, tissue sections were thawed for 10 min, heated at 37° C. for 30 min, blocked for 1 hour in PBS containing 4% normal goat serum (NGS) in the presence (permeabilized) or absence (non-permeabilized) of 0.3% Triton-X100. Patient-derived rAbs were used at 10-20 μg/mL and commercial antibodies were used at the concentrations noted above. Primary antibodies were applied to mouse tissues for 16 hours at 4° C. in PBS containing 4% NGS+/−0.3% Triton-X100. Sections were washed with PBS (for 3 min, 3 times), and Alexa Fluor® 488 anti-human IgG (Life Technologies; 1:500 in PBS containing 4% NGS and 0.3% Triton-X100) was applied. After 2 hours at room temperature and three 3-minute washes in PBS, the slides were mounted using ProLong® Gold (Life Technologies) containing DAPI. In some experiments, brain sections were first blocked and incubated under non-permeabilized conditions for human IgG, albumin, or fibrinogen, then fixed in 4% paraformaldehyde for 10 min, washed 3× in PBS, permeabilized and reblocked in the presence of Triton X-100. Sections were then incubated with primary antibody against rat anti-AQP4 (astrocytes) or claudin-5 (BMECs), washed, and further visualized with the appropriate secondary fluorophore-conjugated antibody as described above. Images were visualized on a Nikon E800 fluorescence microscope using a ×40 or ×60 objective lens, and processed using AxioVision 4.8 software. Confocal images were acquired on an Olympus Fluoview 1000 (Tokyo, Japan) and processed using FV10-ASW software (Olympus).

All animal protocols and procedures were approved by the Animal Use and Care Committee of the University of Colorado Denver and conform to NIH guidelines.

### High Content and High Throughput Imaging Assay Using the Operetta Imager and Harmony Software

10,000 cells were plated per well of a CellCarrier 96-well collagen coated plate (PerkinElmer), followed by immunostaining for NF-κB p65 or ICAM-1 as described above. Plates were scanned and images collected with OPERETTA HTS imaging system (PerkinElmer) at 20× magnification with 13 fields of view/well, equivalent to between 800-1000 cell events. Images were then analyzed with Harmony software (PerkinElmer). Data are mean of triplicates.

### Living-Cell Imaging

Cells were cultured on 3.5 cm glass bottom culture dishes (MatTek dish) and labeled 5 μg/ml of anti-GRP78 Ab (SantaCruz, sc-376768; Mix-n-Stain CF 568A Antibody labeling kits, Sigma-aldrich, MX568S100) for an hour. After 10 minutes exposure of CellMask™ Deep Red Plasma membrane Stain (Life Technology, C10046) and Hoechst 33342 (Life Technologies H3570), images were captured in living cells.

### Preparation of Cell Line Homogenates and Western Blot

U87MG and OL cells were harvested by scrapping in PBS, collected by centrifugation, and then homogenized in a Tris-HCl buffer (0.1 M Tris, pH 8.0, with 1% Triton X-100) containing protease inhibitors (Complete Tablets, Roche Diagnostics) followed by centrifugation at 14,000×g for 30 min at 4° C. The supernatants were collected and stored at −80° C. Total protein concentration in each homogenate was determined by BCA assay kit (Pierce Biotechnology).

Forty micrograms of total protein from U87 and OL cell homogenates were resolved using 4-15% gradient SDS-polyacrylamide gel electrophoresis (Bio-Rad Laboratories, Hercules, Calif., USA). The proteins from the gels were transferred to PVDF (polyvinylidene difluoride) membrane (Millipore, Bedford, Mass., USA) using a semi-dry transfer system (Bio-Rad Laboratories). The PVDF membranes were blocked with 1× Casein Solution (Vector Laboratories) in 1× Tris-buffered saline (TBS) (Bio-Rad Laboratories) and incubated with human rAbs antibodies (10-20 ug/ml) in casein block overnight at 4° C. After washing three times with TBST (Tris-buffered saline containing 0.02% Tween 20), the membranes were incubated with HRP-conjugated goat-antihuman IgG at 25° C. for 2 hours. After three washes with TBST, the blot was transferred to PBS. Protein bands on the membrane were visualized with chemiluminescence (SuperSignal West Pico Chemiluminescent Substrate, Thermo Scientific) and analyzed with an Alpha Innotech FluorchemQ. For Western blot verification of GRP78, purified GRP78 protein obtained from Origene (100 ng/well) and Novus Biologicals (0.5 ug/well) was subjected to sodium dodecyl sulfate polyacrylamide electrophoresis and transferred to PVDF as described above with the exception that after transfer, the PVDF was incubated with 0.5% gluteraldehyde in phosphate buffer saline (PBS) for 15 min, with 0.25 M Tris, pH 7.5 in PBS for 15 min and then washed 3 times in TBS for 10 min before blocking.

### Crosslinking and Mass Spectrometry of AQP4 Seronegative Antigen

Protein A/G beads, disuccinimidyl suberate (DSS), and 3,3′dithiobis-(sulfosuccinimidylpropionate) (DTSSP) were all obtained from Thermo Scientific. U87MG cells were incubated with 20 ug/ml of AQP4 seronegative rAbs in 1× HEPES buffered saline ((HBS) Sigma-Aldrich) containing 0.4 M sucrose, 2 mM MgCl2 and CaCl2 at room temperature (RT) for an hour with occasional rocking. After washing twice with the same solution, the cells were incubated with 12.5 mgs of DTSSP in the same buffer for 30 min at RT. Tris HCl, pH7.5 was added to 25 mM and incubated at RT for 15 min. Cells were lysed in HBS containing 0.32 M sucrose, 1 mM EDTA, 0.1% Triton X-100 with protease inhibitors. Supernatants were incubated with Protein A/G beads overnight with end-over-end mixing and then washed four times with lx HBS. DSS (1 mg) was added and incubate with end-over-end mixing for 30 minutes at RT. Tris HCl, pH7.5 was added to 25 mM and incubated at RT for 15 min. The Protein A/G beads were washed twice in 1× HBS and the antigen was eluted with 50 mM DTT in TBS (10 mM Tris, pH 7.5, 150 mM Ncl) and analyzed by sodium dodecyl sulfate polyacrylamide electrophoresis and/or Western blot. Gel slices corresponding to the 75 kDa band on the Western blots were excised. Sample preparation and mass spectrometry was performed in the Mass Spectrometry Research Center/Proteomics Core at the University of Colorado Cancer Center as follows. Excised gel pieces were destained in ammonium bicarbonate/50% acetonitrile (ACN) and dehydrated in 100% acetonitrile. Disulfide bonds were reduced by dithiothreitol, and cysteine residues were alkylated with iodoacetamide. Proteins were digested with trypsin. Following digestion, the tryptic mixtures were extracted in 1% formic acid/50% acetonitrile). Samples were analyzed on a linear trap quadropole (LTQ) Orbitrap Velos mass spectrometer (Thermo Fisher Scientific, Waltham, Mass., USA) coupled to an Eksigent nanoLC-2D system (Framingham, Mass., USA) through a nanoelectrospray LC-MS interface using a 90-minute gradient from 6 to 40% ACN. Peptide fragmentation was performed in a higher energy collisional dissociation cell with normalized collision energy of 40%, and tandem mass spectra were acquired in the Orbitrap mass analyzer. Data acquisition was performed using Xcalibur software (version 2.0.6; Waltham, Mass., USA). Tandem mass (MS/MS) spectra were converted into mgf files using an in-house script. Mascot (version 2.2; Matrix Science Inc., London, UK) was used to perform database searches against the Swiss-Prot database. Scaffold (version4, Portland, Oreg., USA) was used to validate MS/MS based peptide and protein identifications. Peptide identifications were accepted at a >95.0% probability, protein identifications at a >99.0% probability.

Solute Permeability with 10 kDa Dextran

Effects of NMO-rAb on BBB integrity were assessed by measuring concomitant paracellular permeability (luminal to abluminal) to FITC-labeled 10 kDa dextran or human NMO rAb. Monolayer BMECs were cultured on 24-well collagen-coated Transwell™ tissue culture inserts (0.4 μm pore size, Corning Inc.) for 3 days at 33° C., then for 3 days at 37° C. Cell monolayers were exposed to 50 μg/ml of NMO rAb or control rAb for 24 hours at 37° C. on luminal side. Subsequently, abluminal samples were collected and replaced with equal volume of fresh media. Solute permeability was assessed using FITC-dextran fluorescence. FITC-Dextran (10 kDa, 1 mg/mL final concentration; Sigma Aldrich) was added to upper insert and 5 μl of medium was collected from the lower chamber over 40 min. Aliquots were diluted to 1 ml with PBS. 100 μL of each diluted sample was transferred into 96-well black plates and the fluorescent signals were measured at 490/520 nm absorption/emission wavelengths using a SpectraMax M3e microplate reader (Molecular Devices). For IgG permeability, each rAb was incubated in the upper chamber for 18 hours (the concentration of each rAb was 50 μg/ml; 100 μl conditioned-medium including rAb was incubated in the upper chamber and 200 μl of PBS was added to the lower chamber. Then, PBS in the lower chamber was collected and the IgG concentration within the lower chamber was measured using Easy-Titer Human IgG (H+L) Assay Kit (Thermo Scientific).

### Effect of Commercial GRP78 Antibodies on NF-κB p65 Nuclear Translocation on BMECs

Seven commercial GRP78 Abs (Santa Cruz, sc-376768, sc-1051, sc-1050, sc-13968; Abcam, ab21685, ab12223; Sigma G8918) were tested. Goat-IgG, rabbit-IgG and mouse-IgG (Santacruz, sc-3887, sc-2027, sc-2025) were used as controls. Cultured cells were incubated with these Abs or IgGs (10, 20, 40 and 80 μg/ml) for an hour and fixed with 4% paraformaldehyde.

Removal of Anti-GRP78 Antibodies from Pooled NMO-IgG

Plasmids containing the cDNAs for FLAG-tagged murine GRP78 or FLAG-tagged murine SNAP25 as a control (both obtained from Origene) were transfected into U87MG cells with Lipofectamine 2000 per manufacturer's protocol. The transfected U87MG cells were collected 40 hours post-transfection, washed in cold PBS, and lysed in 1× RIPA Lysis buffer (Cell Signaling Technology) and centrifuged at 14,000×g for 30 min at 4° C. The supernatants were collected and stored at −80° C. until use. For immunoprecipitation, 200, 150, 100 and 25 μg/ml of pooled NMO-IgG or pooled control-IgG was incubated with 100 μg of either FLAG tagged-GRP or control FLAG tagged-SNAP25 protein for 4 hours at 4° C., and then incubated with 40 μl of anti-FLAG Ab-coupling resin (EZ view Red Anti-FLAG2 Affinity Gel beads, Sigma, F2426) for 2 hours at 4° C. After immuno-complexes were pulled down to remove specific IgG against target, the supernatant was saved for analysis.

### Statistics

Statistical analyses were performed using Prism 6 (GraphPad Software). In analyses that required only a single comparison, either a paired Student t test or unpaired Mann-Whitney test was used to determine statistical significance (two-sided). For analyses requiring multiple comparisons, one-way ANOVA between individual groups were performed using Tukey multiple comparisons test, Dunnett multiple comparisons test, Sidak multiple comparisons test or Holm-Sidak multiple comparisons test. All values are expressed as mean±SEM. Degree of significance between groups is represented as follows: *, p<0.05; **, p<0.01; ***, P<0.001.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-9. (canceled)

10. An isolated antibody or antigen-binding fragment thereof that specifically binds human HSPA5, wherein the antibody or antigen-binding fragment thereof comprises:
(a) a heavy chain variable region (VH) comprising VH complementarity determining regions fCDRs) 1, 2, and 3 with the amino acid sequences set forth in SEQ ID NO:1, SEQ ID NO:2, SEQ ID NO:3, respectively, and a light chain variable region (VL) comprising VL CDRs 1, 2, and 3 with the amino acid sequences set forth in SEQ ID NO:4, SEQ ID NO:5, and SEQ ID NO:6, respectively; or
(b) a VH comprising VH CDRs 1, 2, and 3 with the amino acid sequences set forth in SEQ ID NO:11, SEQ ID NO:12, SEQ ID NO:13, respectively, and a VL comprising VL CDRs 1, 2, and 3 with the amino acid sequences set forth in SEQ ID NO:14, SEQ ID NO:15, and SEQ ID NO:16, respectively,
wherein the antibody or antigen-binding fragment thereof is attached to one or more molecules selected from the group consisting of a detectable agent, a polymer, human serum albumin or a fragment thereof, a radioactive material, a fluorescent substance, a luminescent substance, a hapten, a metal chelate, an enzyme, and a cytotoxic agent, a signal peptide, a secretory peptide, a FLAG-tag, a heterologous signal sequence that is heterologous to the VH CDRs 1, 2, and 3 and the VL CDRs 1, 2, and 3.

11. The antibody or antigen-binding fragment of claim 10, wherein:
(a) the VH consists of an amino acid sequence that is at least 75% identical to the amino acid sequence set forth in SEQ ID NO:7 and the VL consists of an amino acid sequence that is at least 75% identical to the amino acid sequence set forth in SEQ ID NO:8;
(b) the VH consists of an amino acid sequence that is at least 75% identical to the amino acid sequence set forth in SEQ ID NO:17 and the VL consists of an amino acid sequence that is at least 75% identical to the amino acid sequence set forth in SEQ ID NO:18;
(c) the VH consists of an amino acid sequence that is at least 95% identical to the amino acid sequence set forth in SEQ ID NO:7 and the VL consists of an amino acid sequence that is at least 95% identical to the amino acid sequence set forth in SEQ ID NO:8;
(d) the VH consists of an amino acid sequence that is at least 95% identical to the amino acid sequence set forth in SEQ ID NO:17 and the VL consists of an amino acid sequence that is at least 95% identical to the amino acid sequence set forth in SEQ ID NO:18;
(e) the VH consists of SEQ ID NO:7 and the VL consists of SEQ ID NO:8; or
(f) the VH consists of SEQ ID NO:17 and the VL consists of SEQ ID NO:18.

12.-13. (canceled)

14. The antibody or antigen-binding fragment of claim 10, wherein the antibody or antigen-binding fragment is an antibody that comprises a heavy chain and a light chain, wherein:
(a) the heavy chain consists of an amino acid sequence that is at least 75% identical to the amino acid sequence set forth in SEQ ID NO:9 and the light chain consists of an amino acid sequence that is at least 75% identical to the amino acid sequence set forth in SEQ ID NO:10;
(b) the heavy chain consists of an amino acid sequence that is at least 75% identical to the amino acid sequence set forth in SEQ ID NO:19 and the light chain consists of an amino acid sequence that is at least 75% identical to the amino acid sequence set forth in SEQ ID NO:20;
(c) the heavy chain consists of an amino acid sequence that is at least 95% identical to the amino acid sequence set forth in SEQ ID NO:9 and the light chain consists of an amino acid sequence that is at least 95% identical to the amino acid sequence set forth in SEQ ID NO:10;
(d) the heavy chain consists of an amino acid sequence that is at least 95% identical to the amino acid sequence set forth in SEQ ID NO:19 and the light chain consists of an amino acid sequence that is at least 95% identical to the amino acid sequence set forth in SEQ ID NO:20;
(e) the heavy chain consists of SEQ ID NO:9 and the light chain consists of SEQ ID NO:10; or
(f) the heavy chain consists of SEQ ID NO:19 and the light chain consists of SEQ ID NO:20.

15.-22. (canceled)

23. An expression vector comprising a promoter operably linked to a polynucleotide encoding a polypeptide comprising:
(i) an immunoglobulin heavy chain variable region (VH) comprising VH complementarity determining regions (CDRs) 1, 2, and 3 with the amino acid sequences set forth in SEQ ID NOs:1-3, respectively, wherein the VH when paired with a light chain variable region (VL) comprising the amino acid sequence set forth in SEQ ID NO:8 binds human HSPA5;
(ii) an immunoglobulin VL comprising VL CDRs 1, 2, and 3 with the amino acid sequences set forth in SEQ ID NOs:4-6, respectively, wherein the VL when paired with a VH comprising the amino acid sequence set forth in SEQ ID NO:7 binds to human HSPA5;
(iii) an immunoglobulin VH comprising VH CDRs 1, 2, and 3 with the amino acid sequences set forth in SEQ ID NOs:11-13, respectively, wherein the VH when paired with a VL comprising the amino acid sequence set forth in SEQ ID NO:18 binds human HSPA5; or
(iv) an immunoglobulin VL comprising VL CDRs 1, 2, and 3 with the amino acid sequences set forth in SEQ ID NOs:14-16, respectively, wherein the VL when paired with a VH comprising the amino acid sequence set forth in SEQ ID NO:17 binds to human HSPA5.

24. The expression vector of claim 23, wherein the promoter is a heterologous promoter.

25. The expression vector of claim 24, wherein the heterologous promoter is a cytomegalovirus, simian virus 40, or retroviral promoter.

26.-27. (canceled)

28. The expression vector of claim 23, wherein the polypeptide comprises a signal peptide.

29. (canceled)

30. The expression vector of claim 23, wherein the polypeptide comprises:
(i) an immunoglobulin heavy chain or fragment thereof comprising a VH with the amino acid sequence set forth in SEQ ID NO:7;
(ii) an immunoglobulin light chain or fragment thereof comprising a VL with the amino acid sequence set forth in SEQ ID NO:8;
(iii) an immunoglobulin heavy chain or fragment thereof comprising a VH with the amino acid sequence set forth in SEQ ID NO:17; or
(iv) an immunoglobulin light chain or fragment thereof comprising a VL with the amino acid sequence set forth in SEQ ID NO:18.

31. The expression vector of claim 23, wherein the polynucleotide comprises the nucleic acid sequence set forth in:
SEQ ID NO:21;
SEQ ID NO:22;
SEQ ID NO:23; or
SEQ ID NO:24.

32. The expression vector of claim 23, wherein the expression vector is a plasmid, phage, virus, or retrovirus.

33. An expression vector comprising:
(a) a first polynucleotide encoding a first polypeptide comprising an immunoglobulin heavy chain or a fragment thereof comprising a heavy chain variable region (VH) comprising VH complementarity determining regions (CDRs) 1, 2, and 3 with the amino acid sequences set forth in SEQ ID NOs:1-3, respectively; and a second polynucleotide encoding a second polypeptide comprising an immunoglobulin light chain or a fragment thereof comprising a light chain variable region (VL) comprising VL CDRs 1, 2, and 3 with the amino acid sequences set forth in SEQ ID NOs:4-6, respectively, wherein the immunoglobulin heavy chain or fragment thereof when paired with the immunoglobulin light chain or fragment thereof forms an anti-human HSPA5 antibody or human HSPA5-binding fragment thereof, or
(b) a first polynucleotide encoding a first polypeptide comprising an immunoglobulin heavy chain or a fragment thereof comprising a VH comprising VH CDRs 1, 2, and 3 with the amino acid sequences set forth in SEQ ID NOs:11-13, respectively; and a second polynucleotide encoding a second polypeptide comprising an immunoglobulin light chain or a fragment thereof comprising a VL comprising VL CDRs 1, 2, and 3 with the amino acid sequences set forth in SEQ ID NOs:14-16, respectively, wherein the immunoglobulin heavy chain or fragment thereof when paired with the immunoglobulin light chain or fragment thereof forms an anti-human HSPA5 antibody or human HSPA5-binding fragment thereof.

34.-39. (canceled)

40. A cDNA comprising a polynucleotide encoding a polypeptide comprising:
(i) an immunoglobulin heavy chain variable region (VH) comprising VH complementarity determining regions (CDRs) 1, 2, and 3 with the amino acid sequences set forth in SEQ ID NOs:1-3, respectively, wherein the VH when paired with a light chain variable region (VL) comprising the amino acid sequence set forth in SEQ ID NO:8 binds human HSPA5;
(ii) an immunoglobulin VL comprising VL CDRs 1, 2, and 3 with the amino acid sequences set forth in SEQ ID NOs:4-6, respectively, wherein the VL when paired with a VH comprising the amino acid sequence set forth in SEQ ID NO:7 binds human HSPA5;
(iii) an immunoglobulin VH comprising VH complementarity determining regions (CDRs) 1, 2, and 3 with the amino acid sequences set forth in SEQ ID NOs:11-13, respectively, wherein the VH when paired with a VL comprising the amino acid sequence set forth in SEQ ID NO:18 binds human HSPA5; or
(iv) an immunoglobulin VL comprising VL CDRs 1, 2, and 3 with the amino acid sequences set forth in SEQ ID NOs:14-16, respectively, wherein the VL when paired with a VH comprising the amino acid sequence set forth in SEQ ID NO:17 binds human HSPA5.

41. A host cell comprising:
(a) a first expression vector comprising a first polynucleotide encoding a first polypeptide comprising an immunoglobulin heavy chain or a fragment thereof comprising a heavy chain variable region (VH) comprising VH complementarity determining regions (CDRs) 1, 2, and 3 with the amino acid sequences set forth in SEQ ID NOs:1-3, respectively, and a second expression vector comprising a second polynucleotide encoding a second polypeptide comprising an immunoglobulin light chain or a fragment thereof comprising a light chain variable region (VL) comprising VL CDRs 1, 2, and 3 with the amino acid sequences set forth in SEQ ID NOs:4-6, respectively, wherein the immunoglobulin heavy chain or fragment thereof when paired with the immunoglobulin light chain or fragment thereof forms an anti-human HSPA5 antibody or human HSPA5-binding fragment thereof; or
(b) a first expression vector comprising a first polynucleotide encoding a first polypeptide comprising an immunoglobulin heavy chain or a fragment thereof comprising a VH comprising VH CDRs 1, 2, and 3 with the amino acid sequences set forth in SEQ ID NOs:11-13, respectively, and a second expression vector comprising a second polynucleotide encoding a second polypeptide comprising an immunoglobulin light chain or a fragment thereof comprising a VL comprising VL CDRs 1, 2, and 3 with the amino acid sequences set forth in SEQ ID NOs:14-16, respectively, wherein the immunoglobulin heavy chain or fragment thereof when paired with the immunoglobulin light chain or fragment thereof forms an anti-human HSPA5 antibody or human HSPA5-binding fragment thereof.

42.-47. (canceled)

48. A method of making an antibody or antigen-binding fragment thereof that specifically binds human HSPA5, the method comprising:
culturing the host cell of claim 41 in a cell culture; and
isolating the antibody or antigen-binding fragment from the cell culture.

49.-50. (canceled)

51. A pharmaceutical composition comprising the antibody or antigen-binding fragment of claim 10.

52. A method of delivering an agent to the brain, spinal cord, or other component of the central nervous system of a human subject in need thereof, the method comprising administering to the human subject the agent and an antibody or antigen-binding fragment thereof that specifically binds human HSPA5.

53.-54. (canceled)

55. The method of claim 52, wherein the antibody or antigen-binding fragment comprises:
(a) a heavy chain variable region (VH) comprising VH complementarity determining regions 1, 2, and 3 with the amino acid sequences set forth in SEQ ID NO:1, SEQ ID NO:2, SEQ ID NO:3, respectively, and a light chain variable region (VL) comprising VL complementarity determining regions 1, 2, and 3 with the amino acid sequences set forth in SEQ ID NO:4, SEQ ID NO:5, and SEQ ID NO:6, respectively; or
(b) a VH comprising VH complementarity determining regions 1, 2, and 3 with the amino acid sequences set forth in SEQ ID NO:11, SEQ ID NO:12, SEQ ID NO:13, respectively, and a VL comprising VL complementarity determining regions 1, 2, and 3 with the amino acid sequences set forth in SEQ ID NO:14, SEQ ID NO:15, and SEQ ID NO:16, respectively.

56. The method of claim 52, wherein the agent is selected from the group consisting of an antibody or antigen-binding fragment thereof, a small molecule, an siRNA, a degron, a protein mimotope, a drug, a prodrug, a chemotherapeutic agent, a ligand binding portion of a receptor, a receptor binding portion of a ligand, an enzyme, a peptide, a protein, a stapled/stitched polypeptide, a nucleic acid, a neurotrophic factor, a growth factor, a neurotransmitter, a neuromodulator, an antibiotic, an antiviral agent, an antifungal agent, an imaging or detectable agent, and a radioisotope.

57. The method of claim 56, wherein the agent is an antibody or antigen-binding fragment thereof selected from the group consisting of:
an antibody or antigen-binding fragment that specifically binds tau,
an antibody or antigen-binding fragment that specifically binds β-amyloid,
an antibody or antigen-binding fragment that specifically binds α-synuclein,
an antibody or antigen-binding fragment that specifically binds TDP-43,
an antibody or antigen-binding fragment that specifically binds AQP4,
an antibody or antigen-binding fragment that specifically binds IL6R,
an antibody or antigen-binding fragment that specifically binds CD20,
an antibody or antigen-binding fragment that specifically binds CD25,
an antibody or antigen-binding fragment that specifically binds VEGF-A,
an antibody or antigen-binding fragment that specifically binds BAFF,
an antibody or antigen-binding fragment that specifically binds alpha-4-integrin,
an antibody or antigen-binding fragment that specifically binds human complement protein C3,
an antibody or antigen-binding fragment that specifically binds human complement protein C1q, and
an antibody or antigen-binding fragment that specifically binds human complement protein C5.

58.-63. (canceled)
